(19) United States
(12) Patent Application Publication
(10) Pub. No.: US 2026/0007737 A1
(43) Pub. Date: Jan. 8, 2026

(54) LIPID POLYSACCHARIDE AMINO ACID NANOPARTICLES AND USE THEREOF

(71) Applicant: GEORGIA TECH RESEARCH CORPORATION, Atlanta, GA (US)

(72) Inventors: Krishnendu ROY, Marietta, GA (US); Bhawana PANDEY, Atlanta, GA (US)

(21) Appl. No.: 18/992,525

(22) PCT Filed: Jul. 10, 2023
(86) PCT No.: PCT/US2023/027265

§ 371 Date: Jan. 8, 2025

Related U.S. Application Data
(60) Provisional application No. 63/359,423, filed on Jul. 8, 2022.

(51) Int. Cl. A61K 39/215; A61K 9/00; A61K 9/51; A61K 31/7105; A61K 31/711; A61K 38/16; A61K 39/00; A61K 45/06; A61K 49/00; A61P 31/14

(52) U.S. Cl. CPC A61K 39/215; A61K 9/0019; A61K 9/0043; A61K 9/5161; A61K 31/7105; A61K 31/711; A61K 38/162; A61K 45/06; A61K 49/0002; A61P 31/14; A61K 2039/5252; A61K 2039/5258; A61K 2039/53; A61K 2039/54; A61K 2039/545; A61K 2039/55555

(57) ABSTRACT

The present disclosure provides for degradable lipid-polysaccharide-based cationic nanoparticles comprising an amino acid such as histidine or arginine conjugated to a C-2 carbon and a lipid conjugated to a C-6 carbon and methods of their use in the delivery of nucleic acids, polynucleotides, siRNA, and/or pDNA and/or hydrophobic drugs.

### II. CROSS-REFERENCE TO RELATED APPLICATIONS

This application claims the benefit of U.S. Provisional Application No. 63/359,423, filed on Jul. 8, 2022, which is incorporated herein by reference in its entirety.

### I. STATEMENT OF GOVERNMENT SUPPORT

This invention was made with government support under Grant No. GR10007030, and U01-AI124270 awarded by the National Institutes of Health. The government has certain rights in the invention.

### III. BACKGROUND

The COVID-19 has made the development of vaccines and therapies an immediate priority worldwide. Even though the current mRNA vaccines have shown excellent efficacy and safety, several limitations have become apparent. These include the breadth and durability of protective immunity, efficacy against emerging variants, inadequate protection from infection and transmission, and lack of mucosal immunity in the lung and nasopharynx. What are needed are new vaccines and adjuvants that can facilitate overcoming these limitations.

### IV. SUMMARY

In accordance with the purposes of the disclosed materials and methods, as embodied and broadly described herein, the disclosed subject matter, in one aspect, relates to nanoparticles and there use in treating disease.

In one aspect, disclosed herein are nanoparticles comprising a polymer having an outer surface and an inner core, wherein the polymer comprises a polysaccharide (such as, for example chitosan), a lipid, and an amino acid (including but not limited to arginine at 0% to 100% by weight of total amino acids and/or histidine at 0% to 100% by weight of total amino acids), wherein the lipid and amino acid are conjugated to the polysaccharide.

Also disclosed herein are nanoparticles of any preceding aspect, wherein the amino acid is conjugated (such as, for example via a disulfide bond) to a C-2 carbon in chitosan.

In one aspect, disclosed herein are nanoparticles of any preceding aspect, wherein the lipid is conjugated to a C-6 carbon in chitosan.

Also disclosed herein are nanoparticles of any preceding aspect, wherein the outer surface of the nanoparticle is hydrophilic and/or wherein the inner core of the nanoparticle is hydrophobic.

In one aspect, disclosed herein are nanoparticles of any preceding aspect, wherein the outer surface of the nanoparticle is loaded with a first agent (such as, for example, a nucleic acid, a polynucleotide, peptide, protein, a siRNA molecule, a miRNA molecule, a shRNA molecule, a pDNA molecule, or any combination thereof including, but not limited to RIG-1, CpG, PUUC, and/or Poly:C) and/or wherein the inner core of the nanoparticle is loaded with a second agent (such as, for example, a small molecule, immune adjuvants, fluorochrome, contrast agents including, but not limited to hydrophobic agents, including but not limited to R848 or MPLA).

Also disclosed herein are nanoparticles of any preceding aspect, wherein the nanoparticle is from 50 nm to 600 nm (such as, for example, 200-250 nm) and/or wherein the nanoparticle has a zeta potential of from +10 mV to +−90 mV (such as, for example, +30 mV to +37 mV).

In one aspect, disclosed herein are vaccines comprising the nanoparticle of any preceding aspect and one or more immunogenic nucleic acids, polynucleotide, peptides, antibody, protein, inactivated virus, killed virus, viral particle, or any combination thereof. For example, the vaccine can comprise a single (i.e., one), 2, 3, 4, 5, 6, 7, 8, 9, or 10 immunogenic nucleic acids, polynucleotides, peptides, proteins, antibodies, viral particles, inactivated viruses, and/or killed viruses, or any combination thereof. When multiple immunogenic nucleic acids, polynucleotides, peptides, proteins, antibodies, viral particles, inactivated viruses, and/or killed viruses, or any combination thereof are present, the nucleic acids, polynucleotides, peptides, proteins, antibodies, viral particles, inactivated viruses, and/or killed viruses, or any combination thereof can be specific for the same or different epitopes (i.e., a multivalent vaccine). Thus, in one aspect, disclosed herein are vaccines of any preceding aspect, wherein the more than one immunogenic nucleic acids, polynucleotides, peptides, proteins, antibodies, viral particles, inactivated viruses, and/or killed viruses are immunogenic against a first epitope. Also disclosed herein are vaccines of any preceding aspect, wherein the more than one immunogenic nucleic acids, polynucleotides, peptides, proteins, antibodies, viral particles, inactivated viruses, and/or killed viruses are immunogenic against a first epitope and at least one second epitope. In one aspect, the first and second epitopes are the same. In another aspect the first and second epitopes are different.

Also disclosed herein are antimicrobial treatment regimens comprising administering one or more vaccines of any of any preceding aspect and/or one or more of the nanoparticles of any preceding aspect and a vaccine.

In one aspect, disclosed herein are antimicrobial treatment regimens, wherein the vaccine comprises one or more immunogenic nucleic acids, polynucleotides, peptides, proteins, antibodies, inactivated viruses, killed viruses, viral particles, or any combination thereof.

Also disclosed herein are antimicrobial treatment regimens of any preceding aspect, wherein the treatment regimen comprises the administration at least two vaccines, a first vaccine and a second vaccine. In one aspect, the vaccine comprises a single immunogenic nucleic acid, polynucleotide, peptide, protein, antibody, viral particle, inactivated virus, or killed virus. In one aspect, the vaccine is multivalent.

In one aspect, disclosed herein are methods of treating, inhibiting, reducing, decreasing, ameliorating, and/or preventing a pulmonary infection (such as, for example, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKUI, HCoV-NL63, SARS-CoV, SARS-CoV-2 (including, but not limited to the SARS-CoV-2 B1.351 variant, SARS-CoV-2B. 1.1.7 (alpha), SARS-CoV-28.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H. and D950N mutations in B.1.621 (Mu), SARS-CoV-2 with G75V, T761, A246-252, L452Q, F490S, D614G, and T859N mutations in C.37 (Lambda), SARS-CoV-2 with T478K, Q498R, and H655Y mutations in B.1.1.529 (Omicron)), or MERS-CoV), Influenza virus A, Influenza virus B, Measles virus, Varicella-Zoster virus, Respiratory syncytial virus, Mumps virus, Adenovirus, Rotavirus, Ebola virus, Marburg virus, Lassa fever virus, Aycobacterium hubercu/osis, Mycobacterium bovis. Mycobacterium bovis s/rain BCG, BCG substrains, Mycobacterium avium, Mycobacterium intracellular, Mycobacterium africanum, Mycobacterium kansasil, Mvcobacterium marinum, Mycobaceriun ulcerans, Mycobacteriun avium subspecies paratuberculosis, Mycobacteriuin chimaera, Bacillus anthracis, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Corynebacterium diphtheriae, Aspergillusfumigahus), comprising administering a therapeutically effective amount of the nanoparticle of any preceding aspect to a patient in need thereof. In some aspects, the nanoparticle is administered via an intramuscular route, an intranasal route, or any combination thereof.

Also disclosed herein in one aspect are methods of making the nanoparticle of any preceding aspect comprising a) carboxylating the polysaccharide; b) thiolating the polysaccharide; c) forming disulfide with a cysteamine; d) conjugating the amino acid using carbodiimide chemistry; e) conjugating stearyl amine using carbodiimide chemistry; f) deprotecting a tert-Butyloxycarbonyl group with trifluoroacetic acid; g) sonicating the nanoparticle; and h) purifying the nanoparticle with dialysis. In some aspect, the method further comprises loading the nanoparticle with the second therapeutic agent.

### V. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A-1D are graphs that demonstrate anti-spike IgA in BAL fluid, anti-spike IgG in sera at various dilutions measured by absorbance at 450 during ELISA assays (FIG. 1 A), neutralizing anti-spike antibody levels (FIG. 1B), quantified at 450 nm in a modified ELISA with biotinylated ACE-2 biotin (FIG. 1C), percentages of cells expressing CD69+CD103+(tissue resident memory T cells) out of CD3+ cells (FIG. 1D). Error bars represent the SEM. P values are *p≤0.05, **p<0.01, ***p<0.001 calculated using Kruskal-Wallis with Dunn's post-hoc test for nonparametric data (FIGS. 1A-1B), two-way ANOVA (FIG. 1C), and one-way ANOVA with Turkey post-hoc test (FIG. 1D).

FIGS. 2A-2H show a synthetic scheme of degradable amphiphilic chitosan polymer. The polysaccharide is modified with disulfide-linked amino acids (Arginine/Histidine) at the —NH2 side (C-2) and with the lipid at the —OH side (C-6). For the polymer synthesis, polysaccharide (chitosan 15KDa) is sequentially modified with synthetic steps starting with (i) carboxylation at the C-6 position using monochloroacetic acid in the basic medium, (ii) thiolation of amine groups at C-2 position with thioglycolic acid using EDC/NHS chemistry, (iii) disulfide bond formation with the thiols (at C-2) and with thiols of cysteamine that generates a disulfide bond and a free amine, (iv) free amine generated after step iii, reacted with the carboxyl group of the N-α Boc protected arginine and histidine amino acids with EDC/NHS chemistry, (v) C6 carboxyl group is reacted with the stearyl amine (lipid chain) using EDC/NHS chemistry in water/ethanol mixture at 80° C., (vi) the last step is the deprotection of N-α Boc group with TFA or dioxane- HCl mixture, followed by dialysis and lyophilization. Purification of all the steps is performed by dialysis or precipitation and followed by lyophilization at the required pH. The polymer was designed uniquely with all the individual functionalities (Arginineihistidine/disulfide/lipid) that enhances the potential for degradable cationic-lipid polymeric nanoparticles to be used for gene/drug delivery applications.

FIG. 3 is a comparison of the 1HNMR spectra of amphiphilic chitosan polymer in DMSO-d⁶ (1) and thiolated Chitosan in D₂O (2). The incorporation of amino acids and stearic acid (lipid) into the chitosan backbone was confirmed by 1HNMR spectroscopy. The appearance of the characteristic peak at 7.1-7.4 ppm (was due to a guanidine functional group present in Arginine amino acids), suggesting the successful grafting of this arginine amino acid to chitosan. Additionally, the characteristic peak of an imidazole ring in acetyl histidine at 8.6 ppm and proton peaks at 7.6 ppm confirm its grafting on the chitosan chain. The peaks at about 1.2 ppm (—CH2—) and 1.6 ppm (—CH3) in the spectrum of amphiphilic chitosan polymer belonging to stearyl groups and confirm the successful incorporation of the stearyl amine in chitosan. The peak respective to the amino acids and the lipids are not present in the thiolated chitosan, which confirms the conjugation for these functionalities in the amphiphilic chitosan polymer.

FIG. 4 is a depiction of cationic and degradable (disulfide linked) lipid polysaccharide-amino acid nanoparticle fabrication procedure using self-assembly method via probe sonication in DMSO/water mixture and further purification via dialysis. The degradable cationic nanoparticles have a size range of-200 nm and a zeta potential of ˜+30 mV. The hydrophobic molecules (R848 or MPLA) adjuvants are loaded during the self-assembly process. The negative charged adjuvants-nucleic acids (CpG/PUUC/Poly L:C) are loaded on charged particle surface by electrostatic interaction. The introduction of individual functionalities included in the amphiphilic polymer is (1) Arginine-which has guanidine groups that help in the strong binding of nucleic acids, and (2) Histidine amino acids which have the buffering effect and help in the endosomal escape process/proton sponge effect which helps in the release of nucleic acids, (3) Disulfide linker (S-S) is introduced between the chitosan and amino acids that help in degradation in a reducible environment and release the nucleic acids from the surface, (4) the hydrophobic lipid chain (18 carbon) which form the strong hydrophobic core for micelles and help in encapsulation of hydrophobic adjuvants (MPLA/R848).

FIG. 5 shows single and combination adjuvant loading on CL-NP and doses for GM-CSF BMDCs activation in-vitro studies and in-vivo studies. Size, PDI and zeta measurements were taken for all NPs prior to electrostatically loading adjuvants CpG or PUUC.

FIGS. 6A, 6I, and 6C show in vitro activation of murine BMDCs with adjuvant-loaded Nanoparticles. BMDC were treated with nanoparticles (12 μg) loaded with R848 adjuvant (20 ng), CpG adjuvant (100ng), PUUC adjuvant (100 ng). At 24 h of treatment, cell-free supematants were harvested and assayed for IL12p70. IL-18, and IFN-S by ELISA. In all experiments, single and dual delivery was performed with a single nanoparticle system. Statistical significance was evaluated with one-way ANOVA followed by Tukey's test for multiple comparisons. *P<0.05, **P<0.01, ****P:5 0.0001. Co-delivery of multiadjuvants chitosan CpG, R848 and PUUC in the form of nanoparticles results in an additive innate immune response from mice BMDC (GM-CSF).

FIGS. 7A, 7B, and 7C show CL-NPs delivered intramuscularly prime and intranasally boost with spike protein enhance humoral responses in serum. Multi-adjuvanated CL-NP's delivered intramuscularly prime and intranasally boost with spike protein enhance T cell responses. Female BALB/c mice were immunized I.M. into both tibialis anterior muscles at day 0 (1st dose) with soluble spike protein at doses of 1000 ng with or without adjuvant-NPs (250ug) loaded with CpG, R848 and PUUC (40ug, 20ug, 20ug), respectively. On day 21, mice received the 2nd dose of protein subunit vaccine I.N with similar doses of formulations except for the CpG dose of 20ug. Mice were euthanized after two weeks on day 36 to collect blood, BAL fluid, and lungs. FIG. 7A shows a comparison of area under the curve (AUC) of anti-spike IgG in post-2nd dose sera at various dilutions measured by ELISA. FIG. 7B shows anti-spike IgG measured by absorbance at 450 nm during ELISA. FIG. 7C shows ACE-2 signal measured by absorbance at 450 nm in spike protein neutralization assay with post-2nd dose sera with ELISA (error bars represent the SEM). Normality was assessed with the Kolmogorov-Smirnov test. Statistical significance was determined with the Kruskal-Wallis test and Dunn's post-hoc test for multiple comparisons. Statistical significance was calculated with One-Way ANOVA and Tukey post-hoc test. *p≤0.05, **p≤0.01, ***p≤0.001, ****p≤0.0001 for all graphs. CpG+PUUC CL-NP's with soluble spike protein formulation generated significantly high systemic antigen-specific IgG responses in mice immunized 1.M prime and IN boost and also detectable at the lower dilution at 1:1000 dilution. Immunized mice with this formulation also contained antibodies neutralizing spike protein, meaning ACE-2 binding was reduced.

FIGS. 8A, 8I, 8C, and 8D show CL-NPs delivered intramuscularly prime and intranasally boost with spike protein enhance humoral responses in serum. Multi-adjuvanated CL-NP's delivered intramuscularly prime and intranasally boost with spike protein enhance T cell responses. Female BALB/c mice were immunized I.M. into both tibialis anterior muscles at day 0 (1st dose) with soluble spike protein at doses of 1000 ng with or without adjuvant-NPs (250ug) loaded with CpG, R848 and PUUC (40ug, 20ug, 20ug), respectively. On day 21, mice received the 2nd dose of protein subunit vaccine 1.N with similar doses of formulations except for the CpG dose of 20ug. Mice were euthanized after two weeks on day 36 to collect blood, BAL fluid, and lungs. FIG. 8A shows the sera were serially diluted and evaluated for anti-spike IgG1 by ELISA. FIG. 8B shows the area under the curve (AUC) for each dilution curve was calculated for each mouse serum sample. FIG. 8C shows anti-spike IgG2a was measured by ELISA and 8D) AUC was calculated and compared for each experimental group with ELISA (error bars represent the SEM). Normality was assessed with the Kolmogorov-Smirnov test. Statistical significance was determined with the Kruskal-Wallis test and Dunn's post-hoc test for multiple comparisons. *p≤0.05, **p≤0.01, ***p≤0.001, ****p S0.0001 for all graphs. Both PUUC and CpG+PUUC CL-NP's with soluble spike protein generated significantly high systemic antigen-specific IgG1 responses in mice immunized I.M prime and IN boost and also detectable at the lower dilution at 1:1000 dilution. But CpG+PUUC CL-NP's with soluble spike protein formulation generated significantly high systemic antigen-specific IgG2a responses, which indicate the preferentially induced T_{H}2-biased response.

FIGS. 9A, 9B, 9C, and 9D show CL-NPs delivered intramuscularly prime and intranasally boost with spike protein enhance humoral responses in BAL fluid. Multi-adjuvanated CL-NP's delivered intramuscularly prime and intranasally boost with spike protein enhance T cell responses. Female BALB/c mice were immunized I.M. into both tibialis anterior muscles at day 0 (1st dose) with soluble spike protein at doses of 1000 ng with or without adjuvant-NPs (250ug) loaded with CpG, R848 and PUUC (40ug, 20ug, 20ug), respectively. On day 21, mice received the 2nd dose of protein subunit vaccine I.N with similar doses of formulations except for the CpG dose of 20ug. Mice were euthanized after two weeks on day 36 to collect blood, BAL fluid, and lungs. FIG. 9A shows anti-spike IgG in BAL fluid of post-2nd dose at 1:5 dilution measured by absorbance at 450 nm with ELISA. FIG. 9B shows anti-spike IgG1 in BAL fluid of post-2nd dose measured by absorbance at 450 nm at 1:5 dilution. FIG. 9C shows anti-spike IgG2a IgG1 in BAL fluid of post-2nd dose measured by absorbance at 450 nm at 1:5 dilution. FIG. 9D shows anti-spike IgA in IgG1 in BAL fluid of post-2nd dose measured by absorbance at 450 nm at 1:5 dilution. Normality was assessed with the Kolmogorov-Smirnov test. Statistical significance was determined with the Kruskal-Wallis test and Dunn's post-hoc test for multiple comparisons. *p5.0.05, **p≤0.01, ***p≤0.001, ****p <0.0001 for all graphs. PUUC and the CpG+PUUC CL- NP's show a high level of IgG but it is comparatively more in CpG+PUUC combination. PUUC with soluble spike protein generated significantly high systemic antigen-specific IgG1 responses in mice immunized IM prime and IN boost. But CpG+PUUC CL-NP's with soluble spike protein formulation generated significantly high systemic antigen-specific IgG2a responses, which indicate the preferentially induced T2-biased response. CL-NP's with the CpG+PUUC combination adjuvants shows a high level of IgA significantly and with comparison to PUUC and other groups.

FIGS. 10A-10F show multi-adjuvanated CL-NP's delivered intramuscularly prime and intranasally boost with spike protein enhance T cell responses. Female BALB/c mice were immunized I.M. into both tibialis anterior muscles at day 0 (1st dose) with soluble spike protein at doses of 1000 ng with or without adjuvant-NPs (250ug) loaded with CpG, R848 and PUUC (40ug, 20ug, 20ug), respectively. On day 21, mice received the 2nd dose of protein subunit vaccine I.N with similar doses of formulations except for the CpG dose of 20ug. Mice were euthanized after two weeks on day 36 to collect blood, BAL fluid, and lungs. Lung cells were restimulated with spike peptide pools for 6 h and stained for analysis by flow cytometry. Percentages of cells expressing 10A) CD3+CD69+CD103+(tissue-resident memory T cells) B) IFNγ+out of CD3+CD69+CD103+ cells, IOC) CD8a+CD69+CD103+(tissue-resident memory T cells), 10D) Granzyme B+out of CD8a+ T cells (10E). CD4+CD69+CD103+(tissue-resident memory T cells), (1° F.) Granzyme B+out of CD4+ T cells. Normality was assessed with the Kolmogorov-Smirnov test. Statistical significance was determined with the Kruskal-Wallis test and Dunn's post-hoc test for multiple comparisons. Statistical significance was calculated with One-Way ANOVA and Tukey post-hoc test. *p≤0.05, **p≤0.01, ***p≤0.001, ****p≤0.0001 for all graphs. CL-NP's with the CpG+PUUC combination adjuvants show a significant increase in CD3+CD69+CD103+ and CD8a+CD69+CD103+(tissue-resident memory T cells) in lung tissues. After restimulation with the spike peptide pools, this combination adjuvant formulation also shows a non-significant increase in CD3+CD69+CD103+ T cells producing IFNγ and CD8a+ T cells producing Granzyme B+. CL-Nps with CpG+PUUC and with the CpG+R848 adjuvants show a non-significant increase in CD4+CD69+CD103+(tissue-resident memory T cells) and CD4+ T cells producing Granzyme B+.

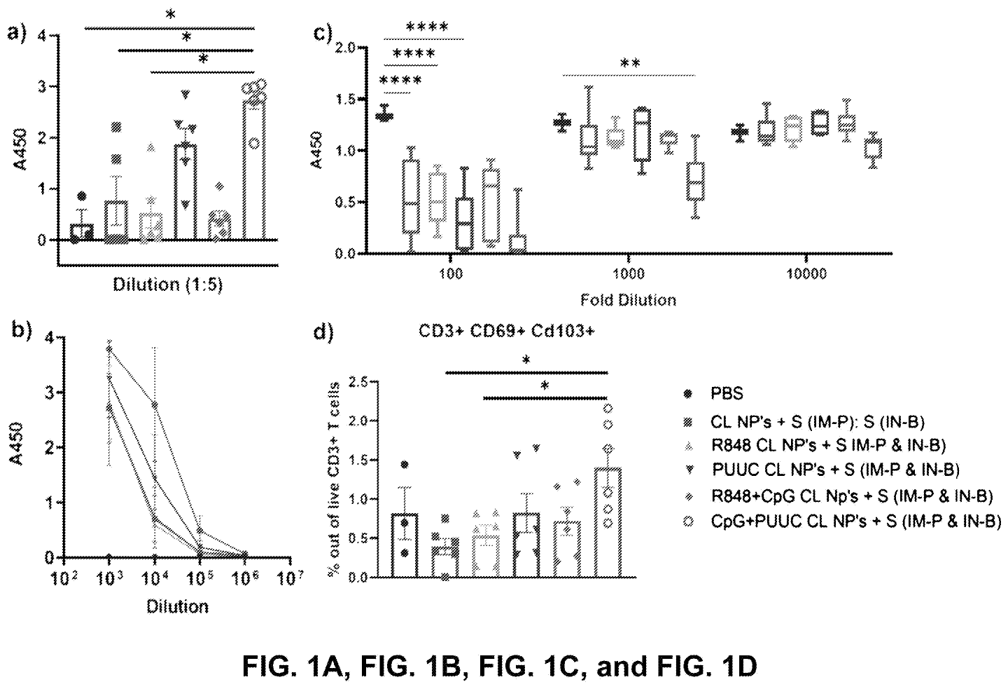

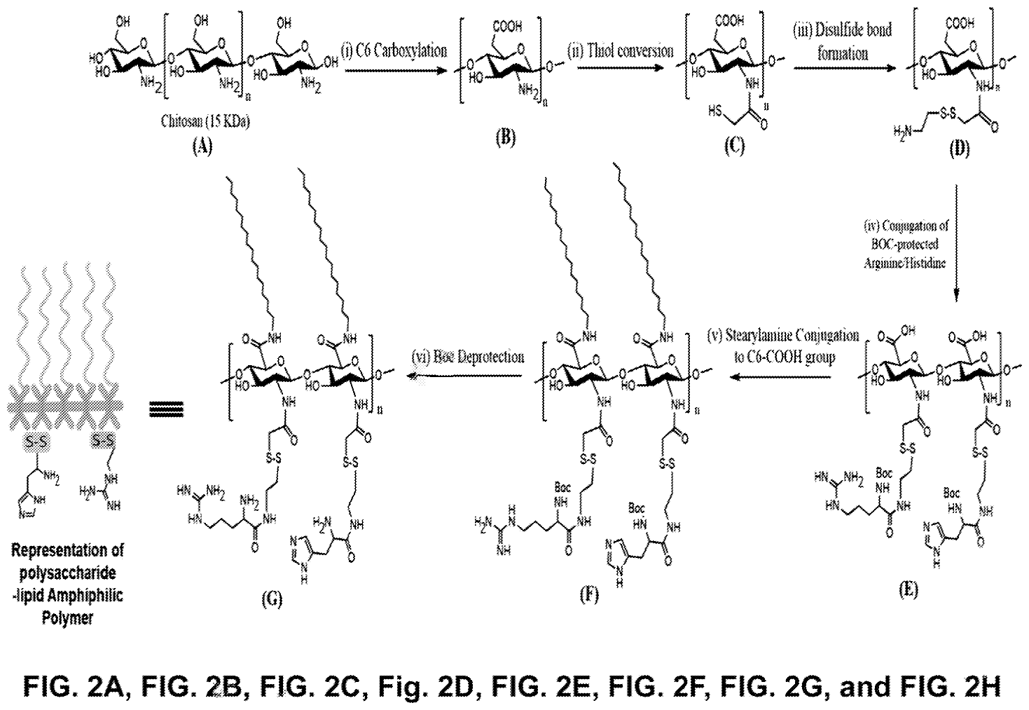

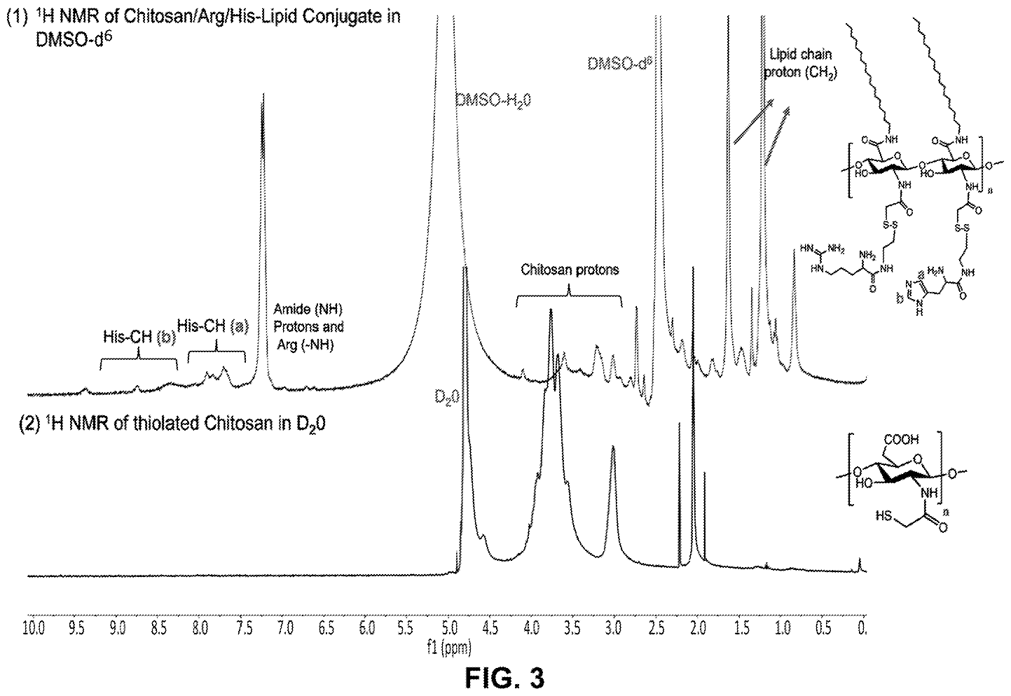

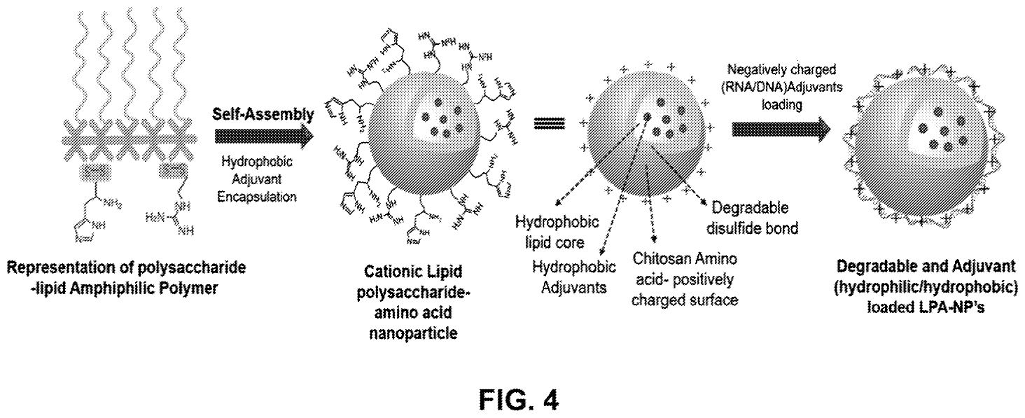

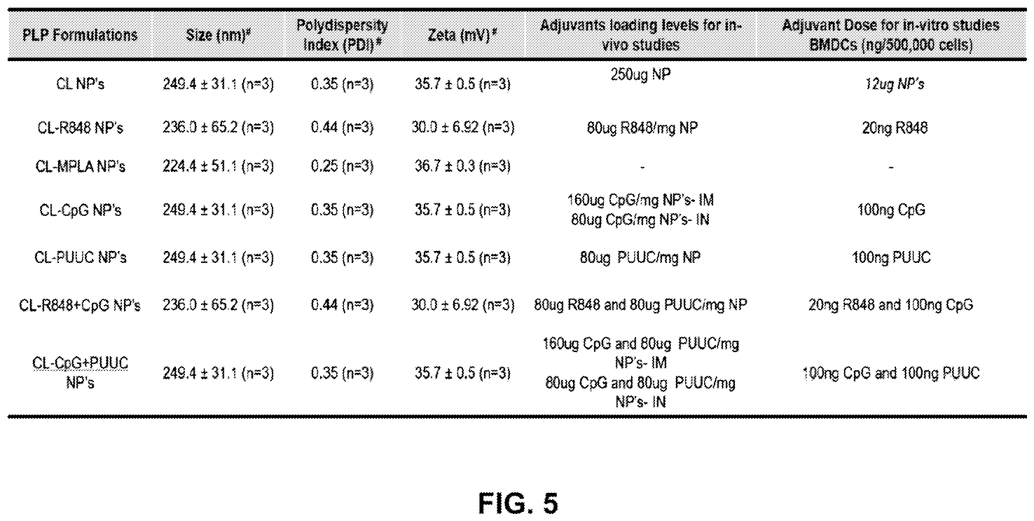

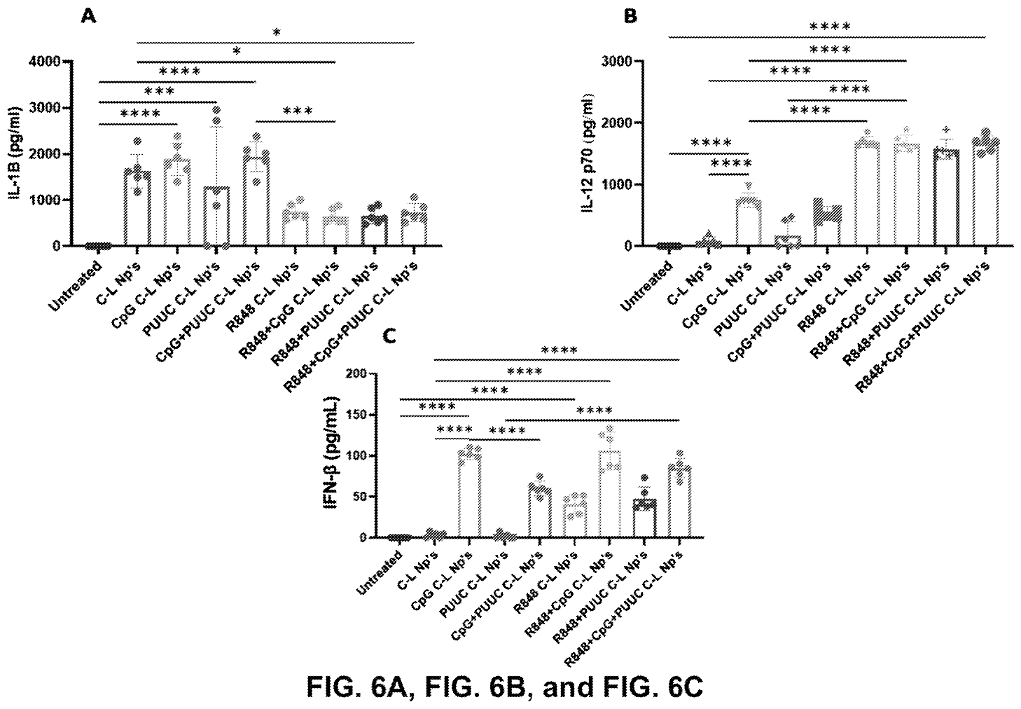

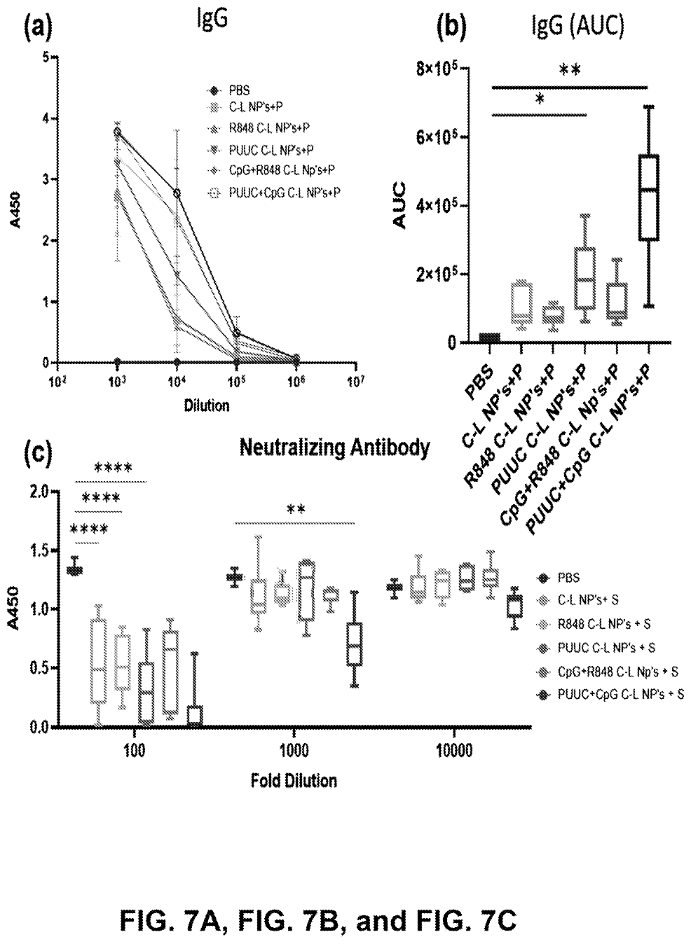

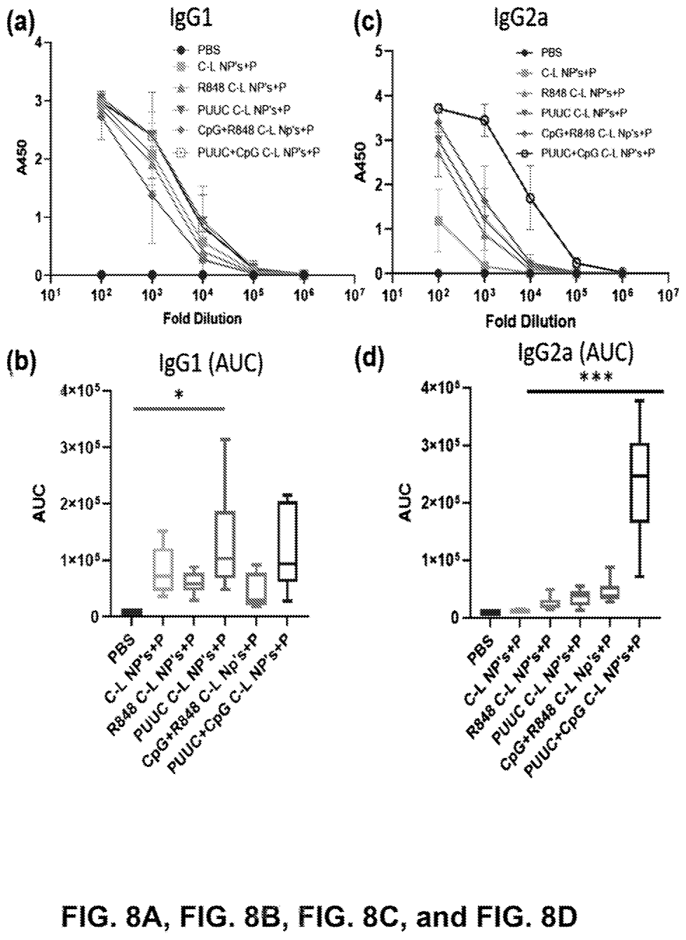

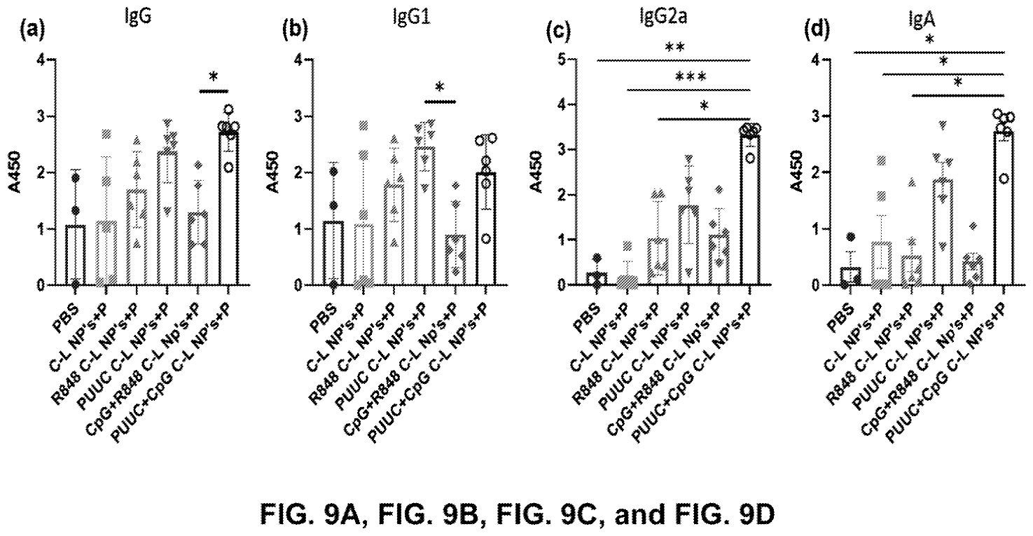

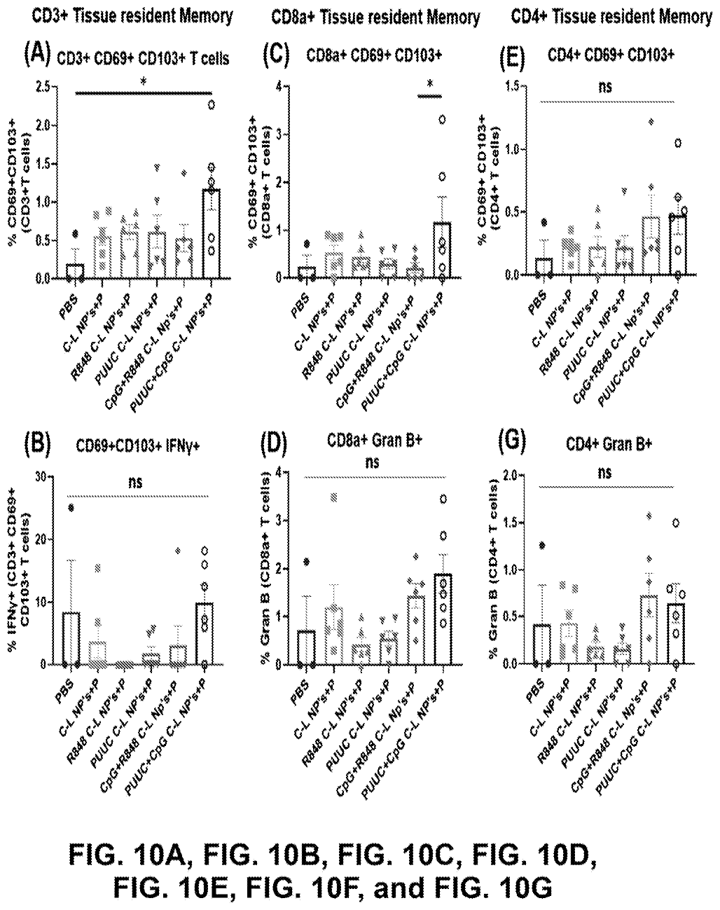

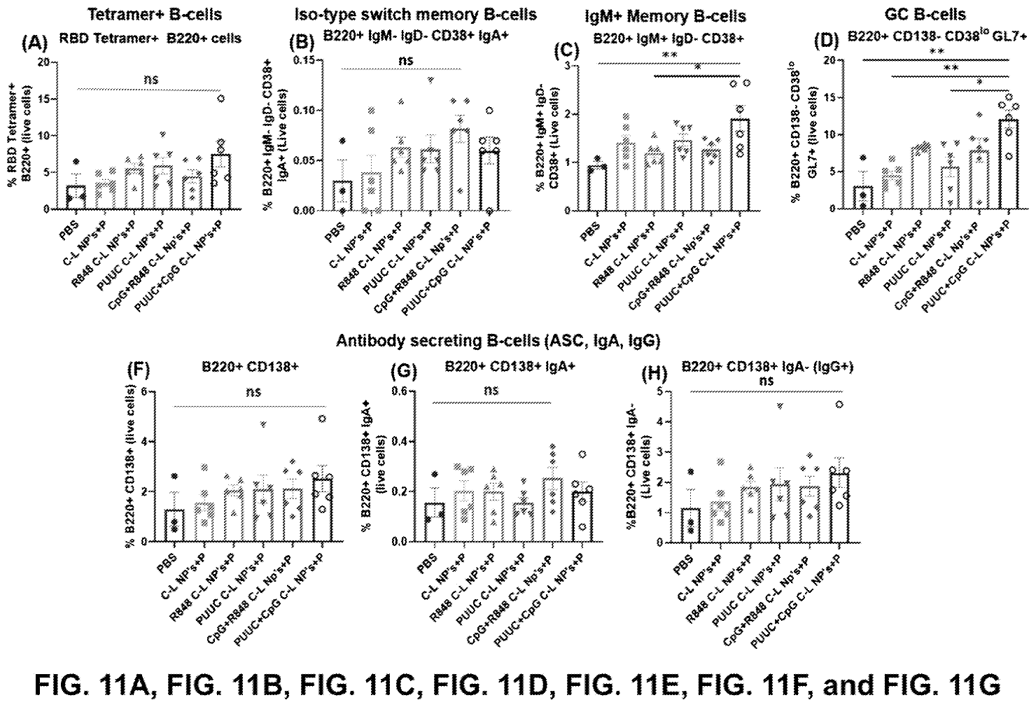

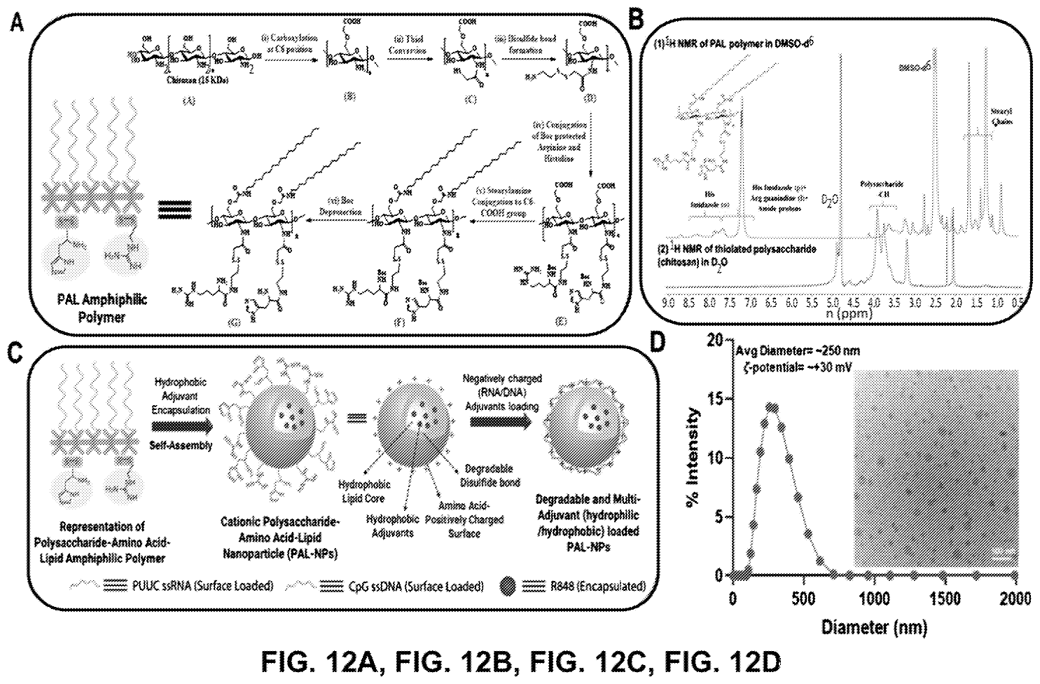

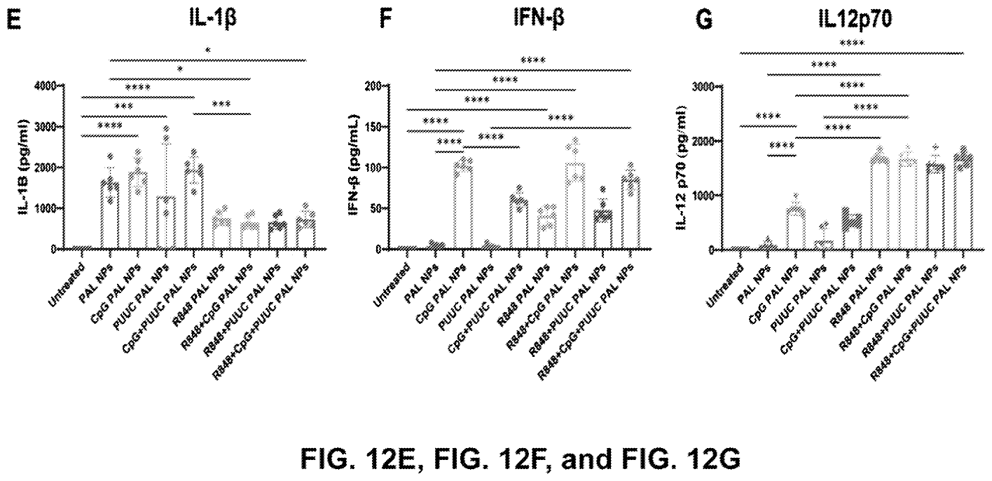

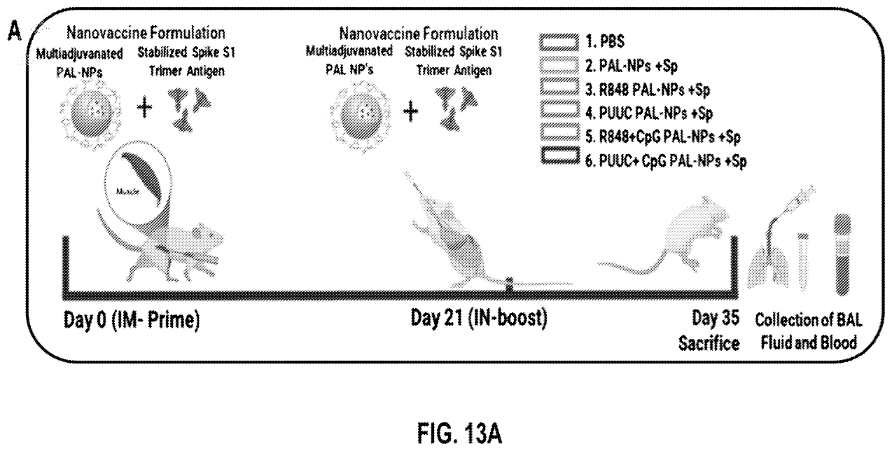

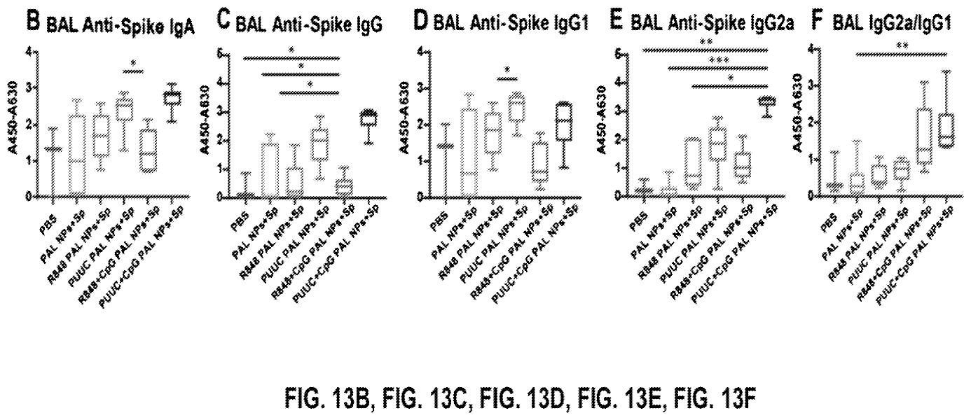

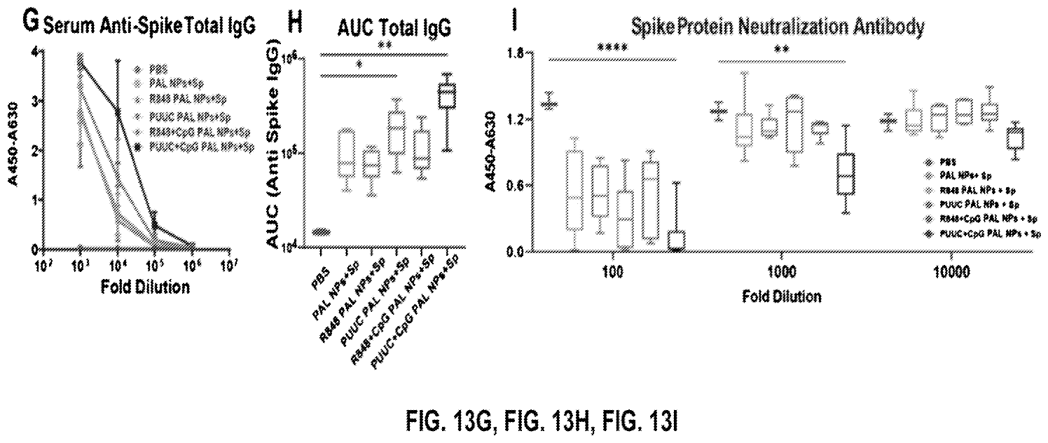

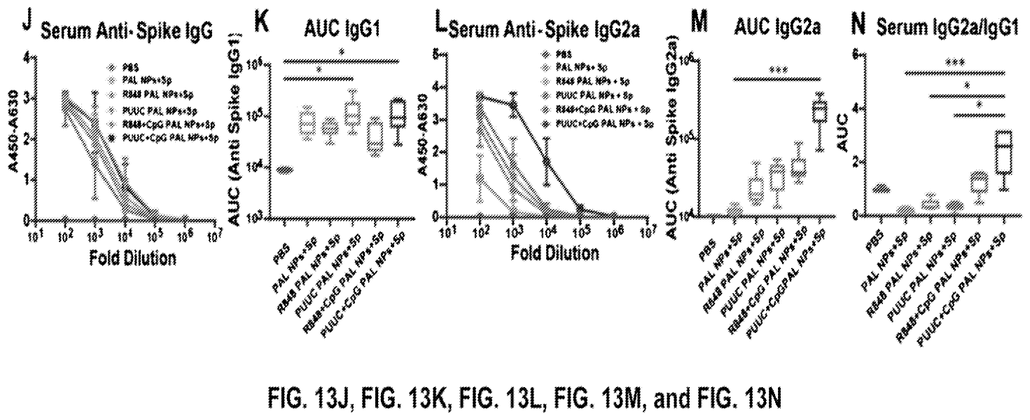

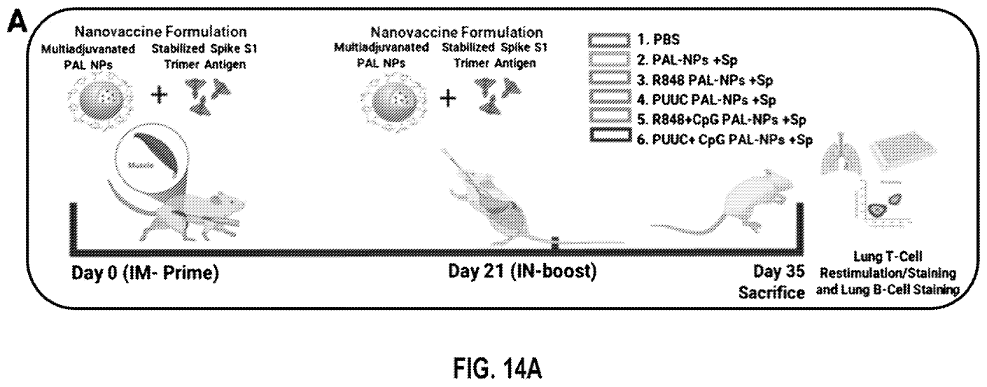

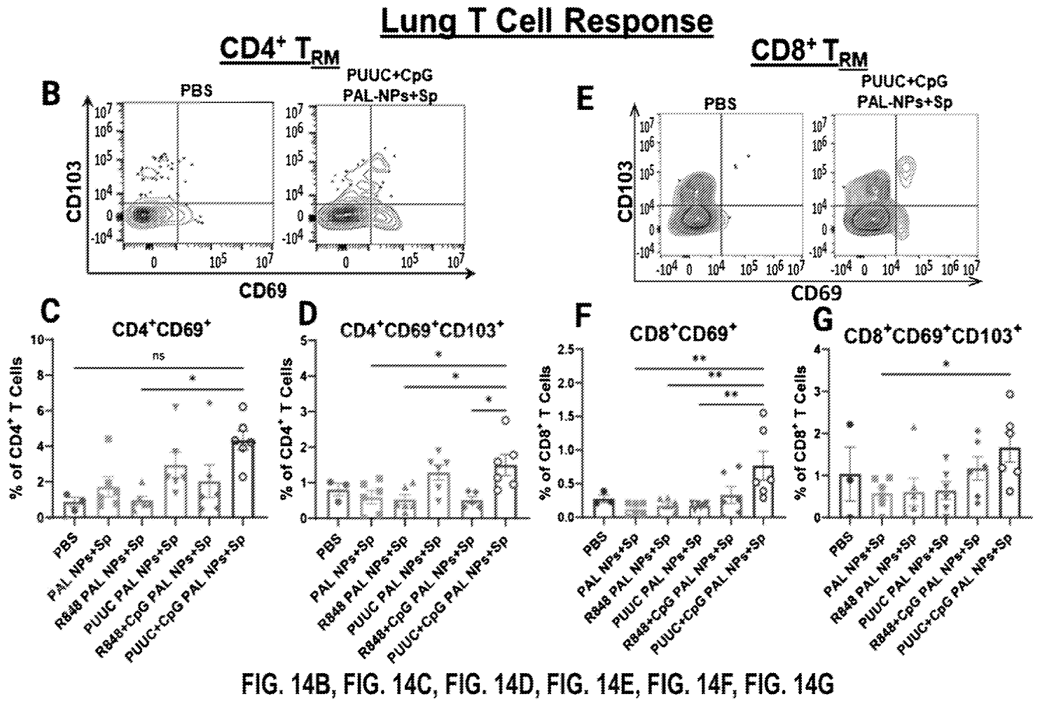

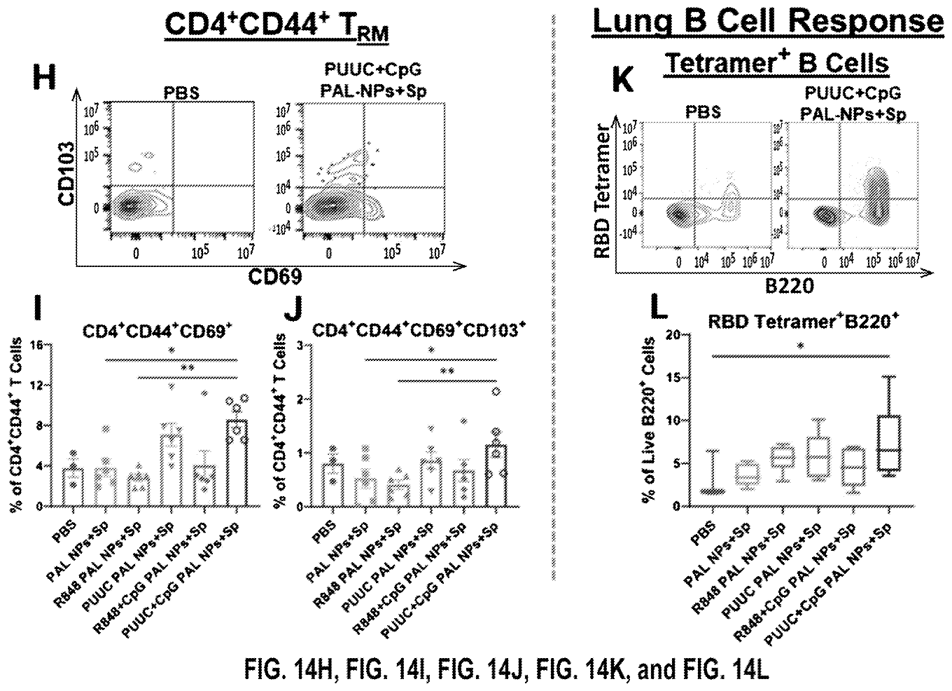

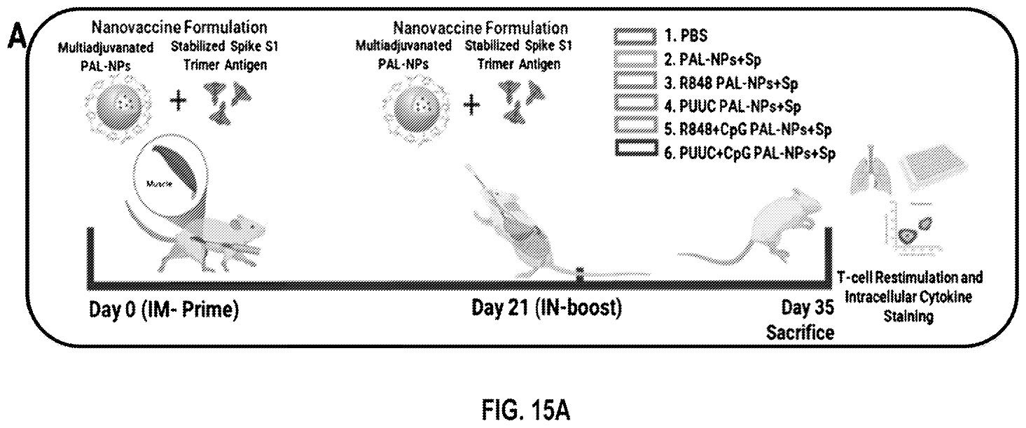

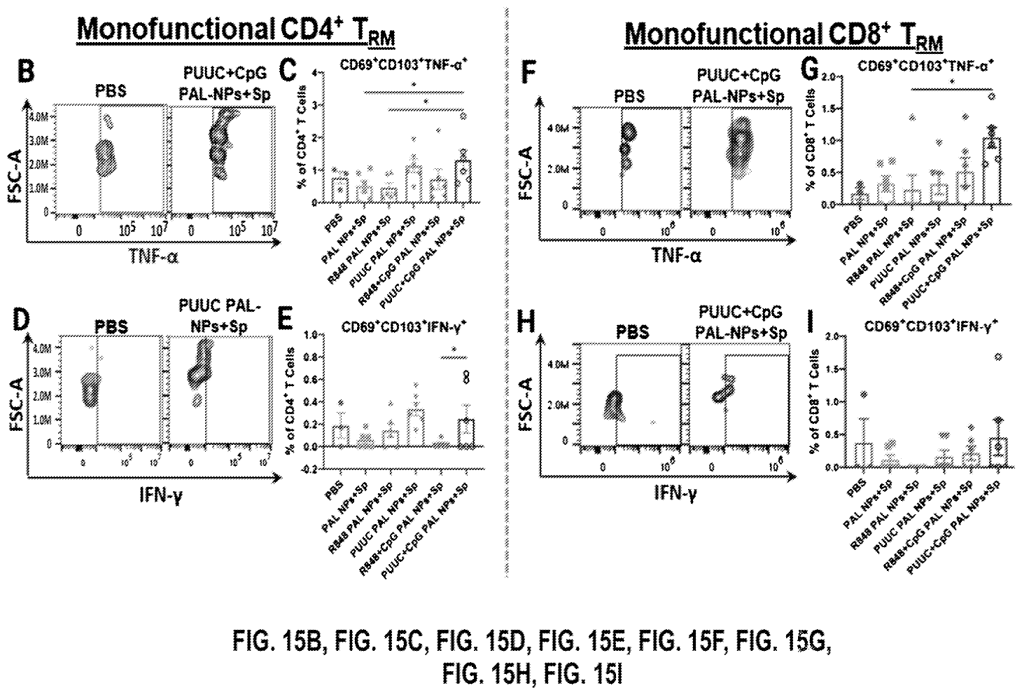

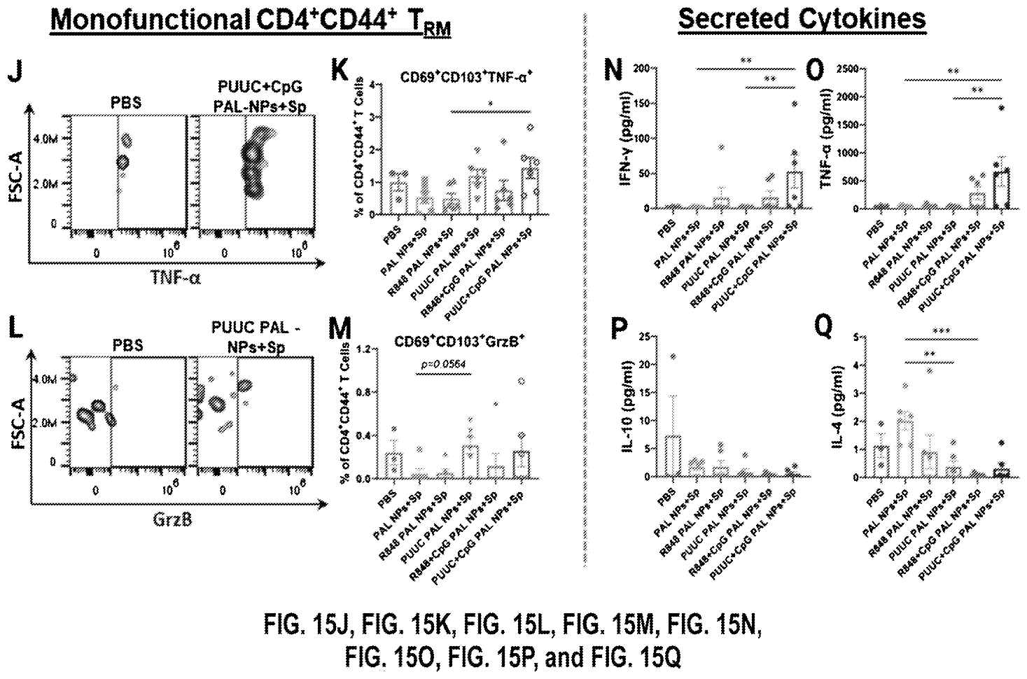

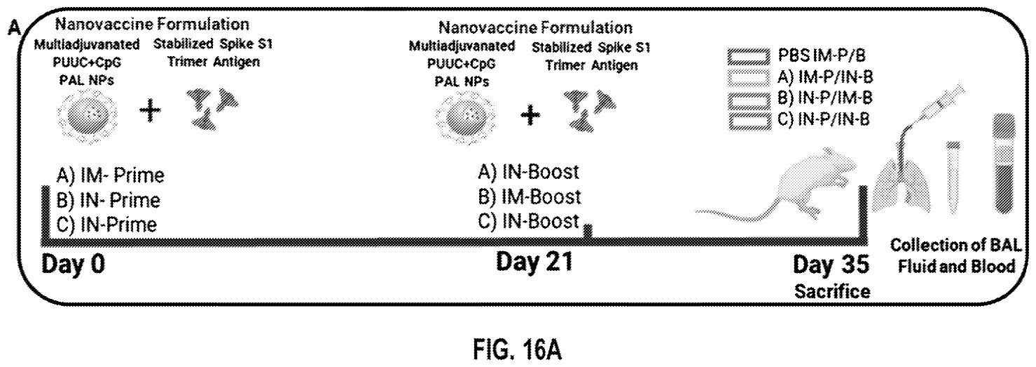

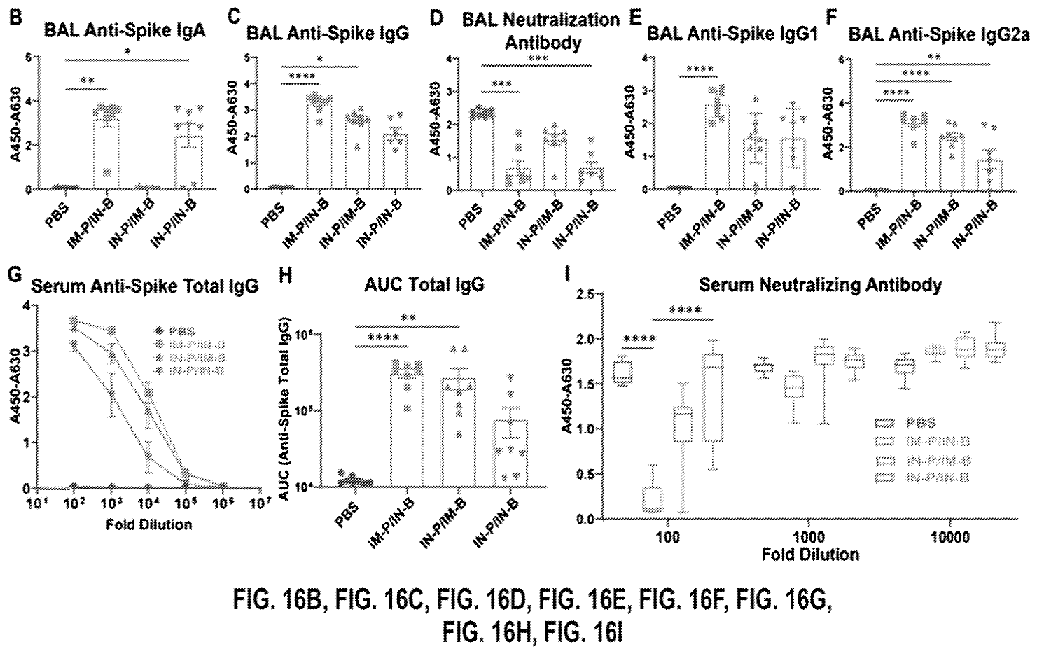

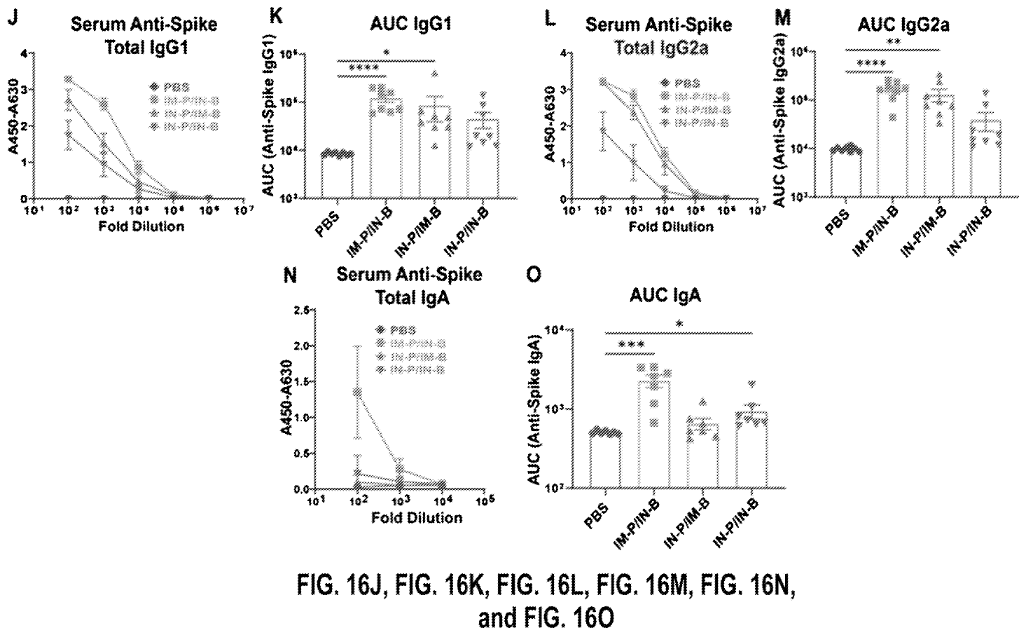

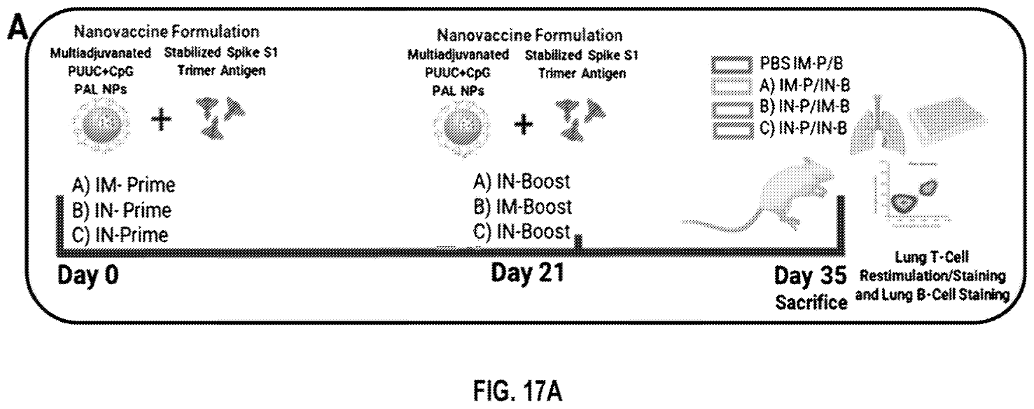

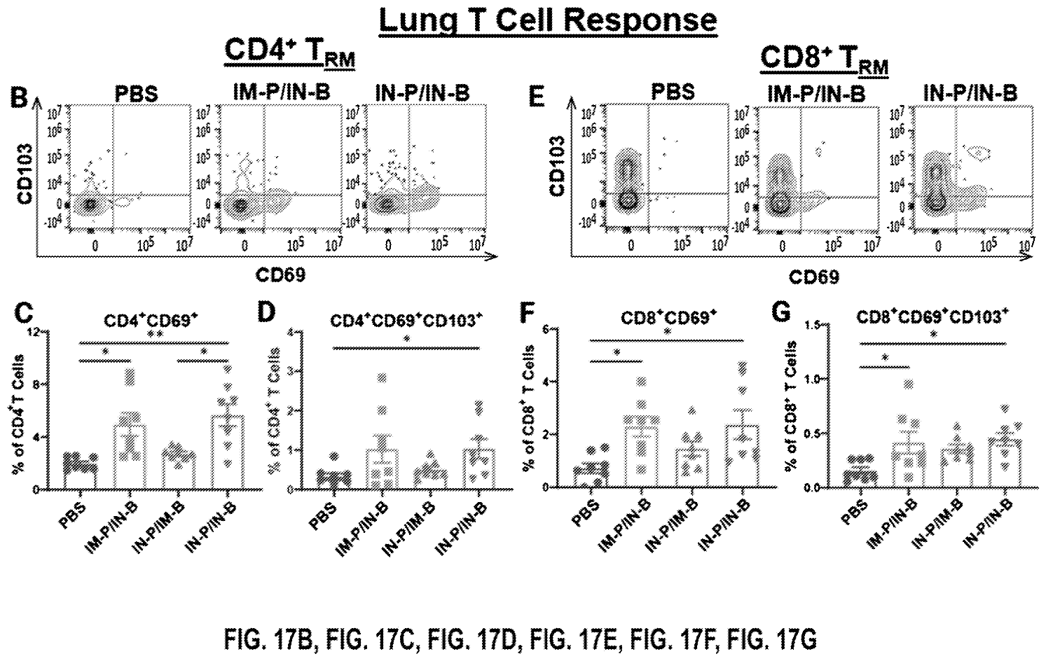

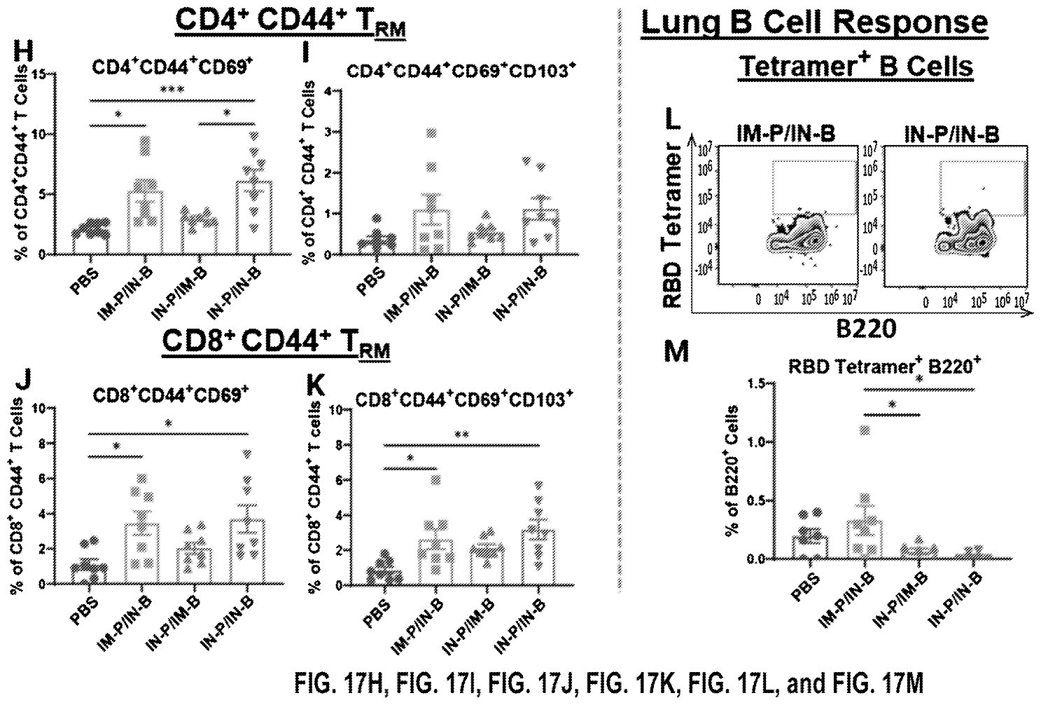

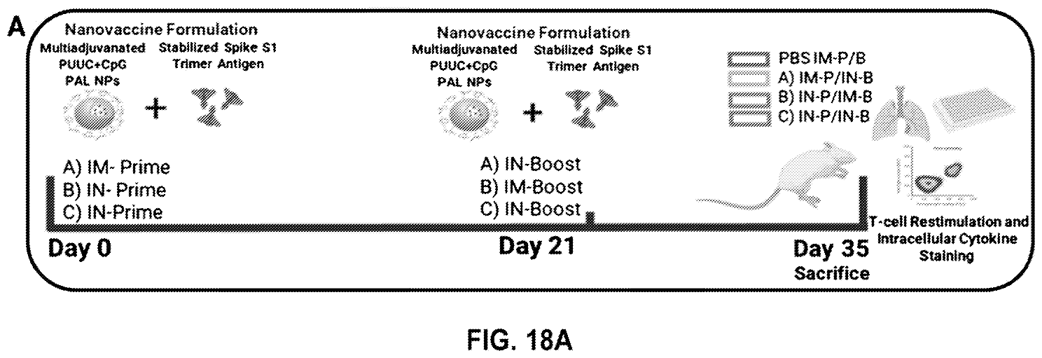

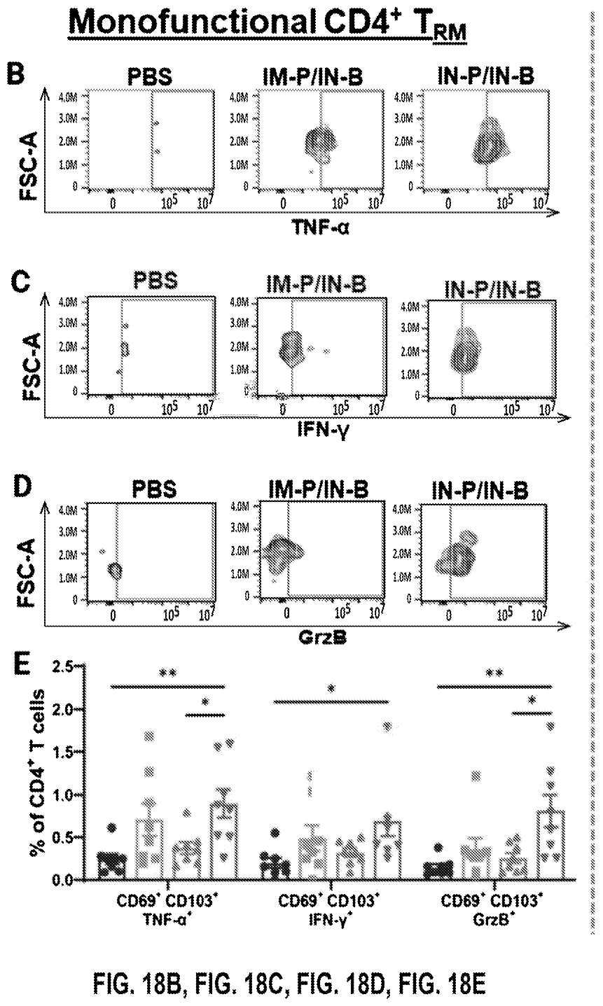

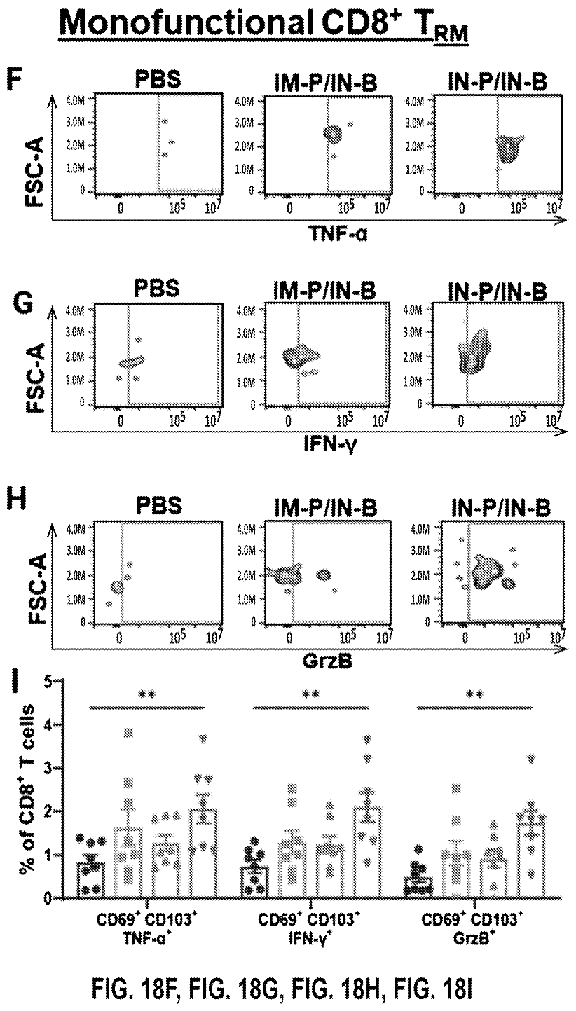

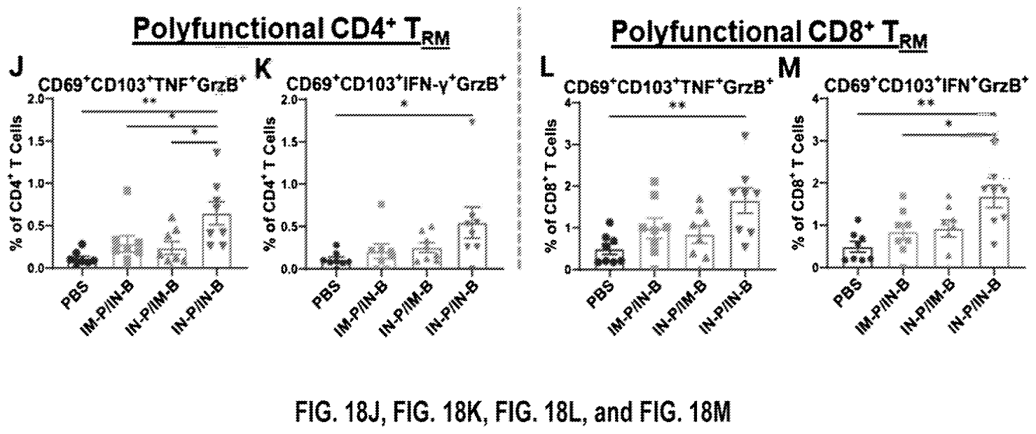

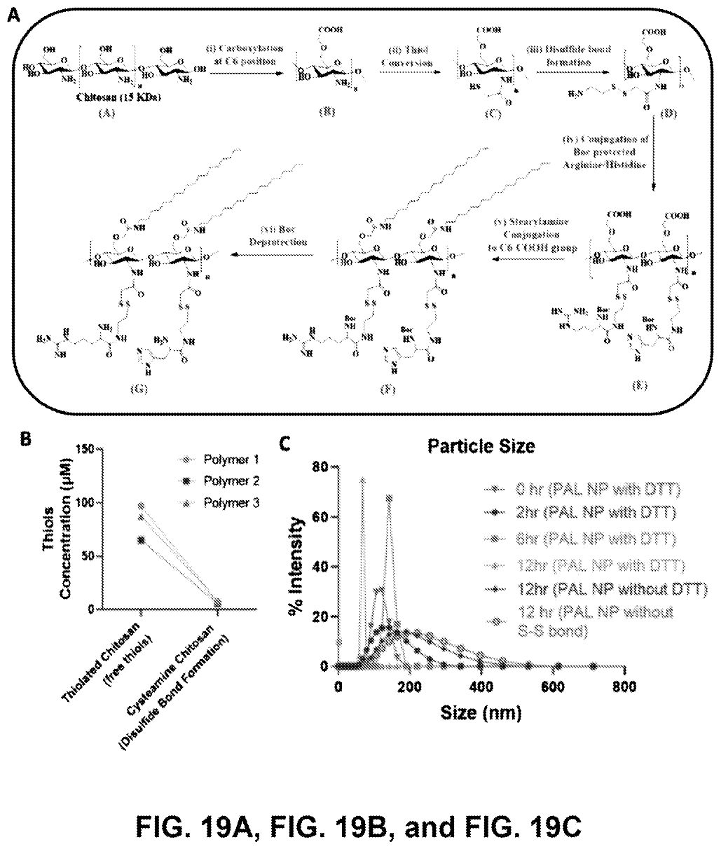

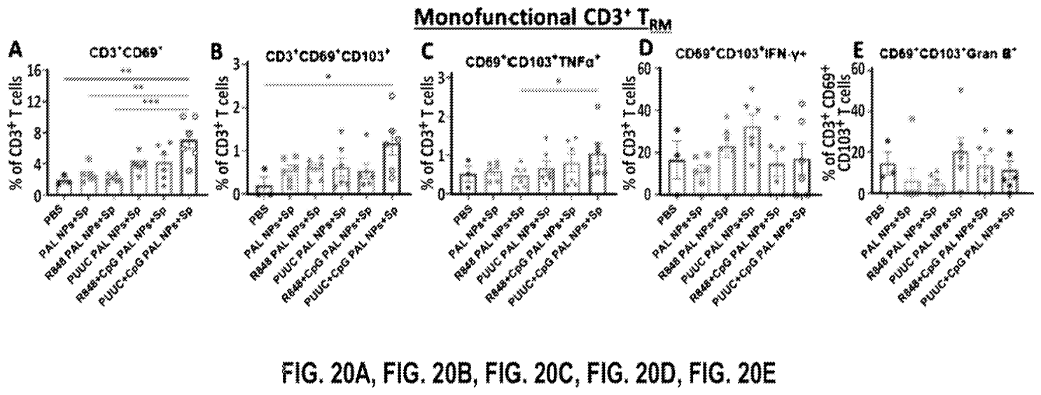

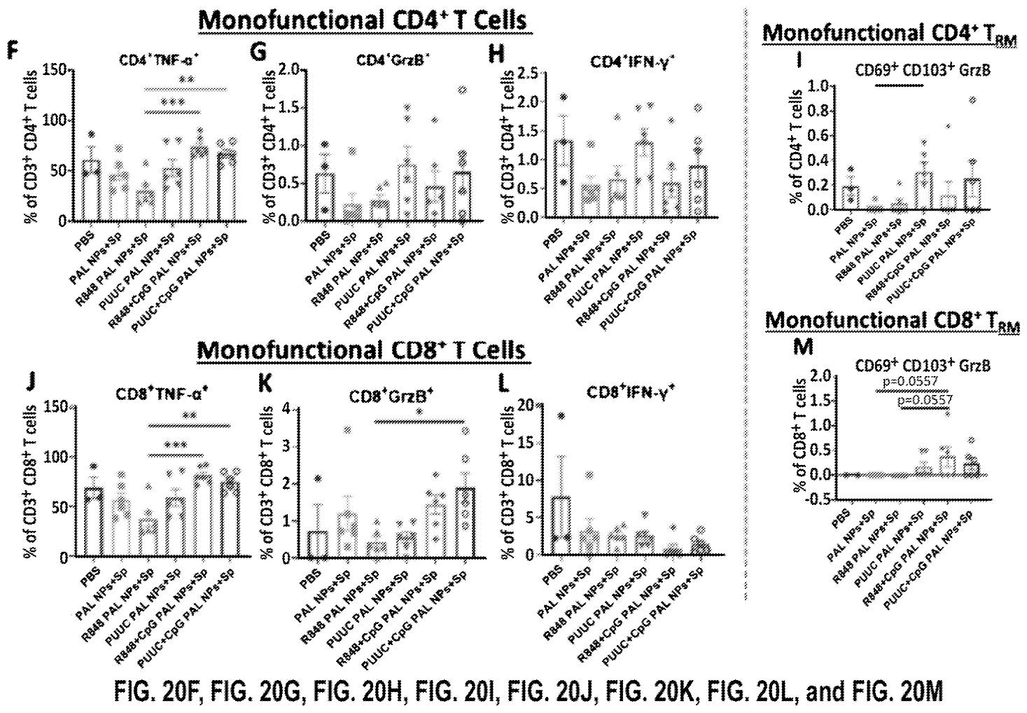

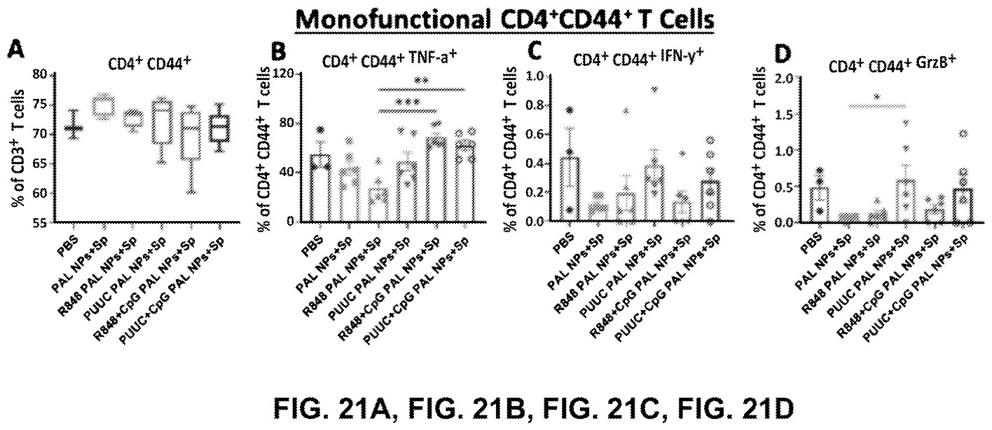

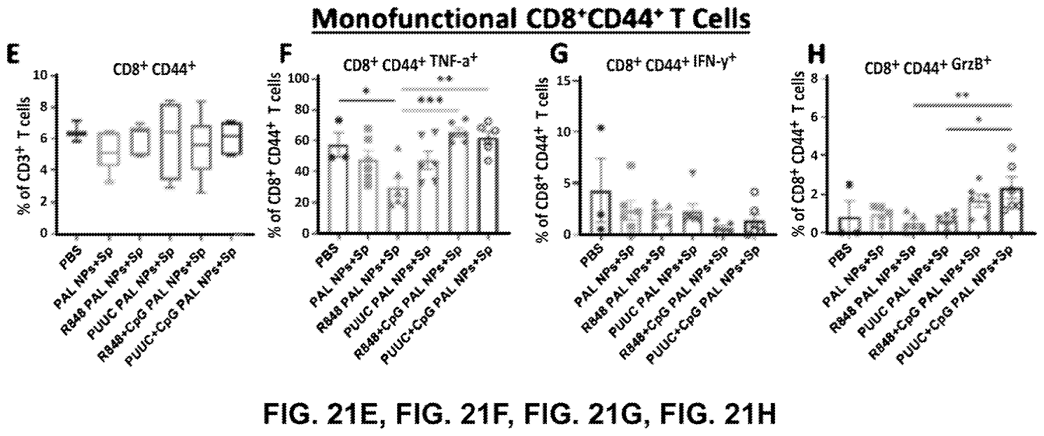

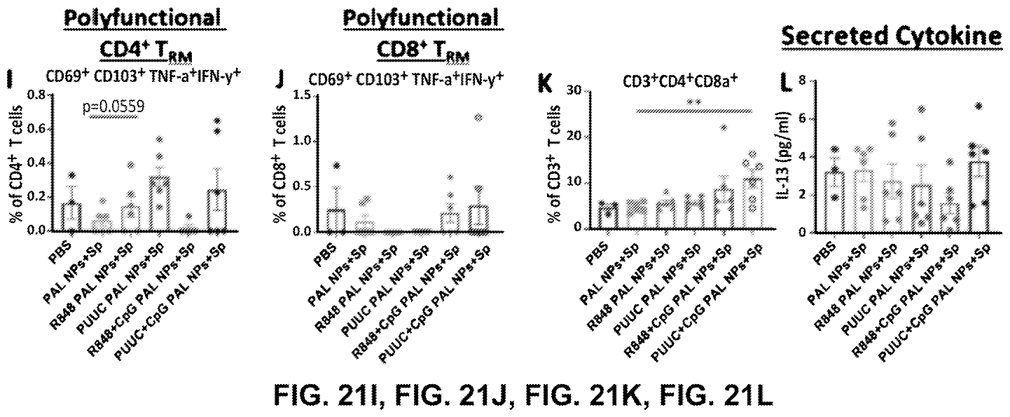

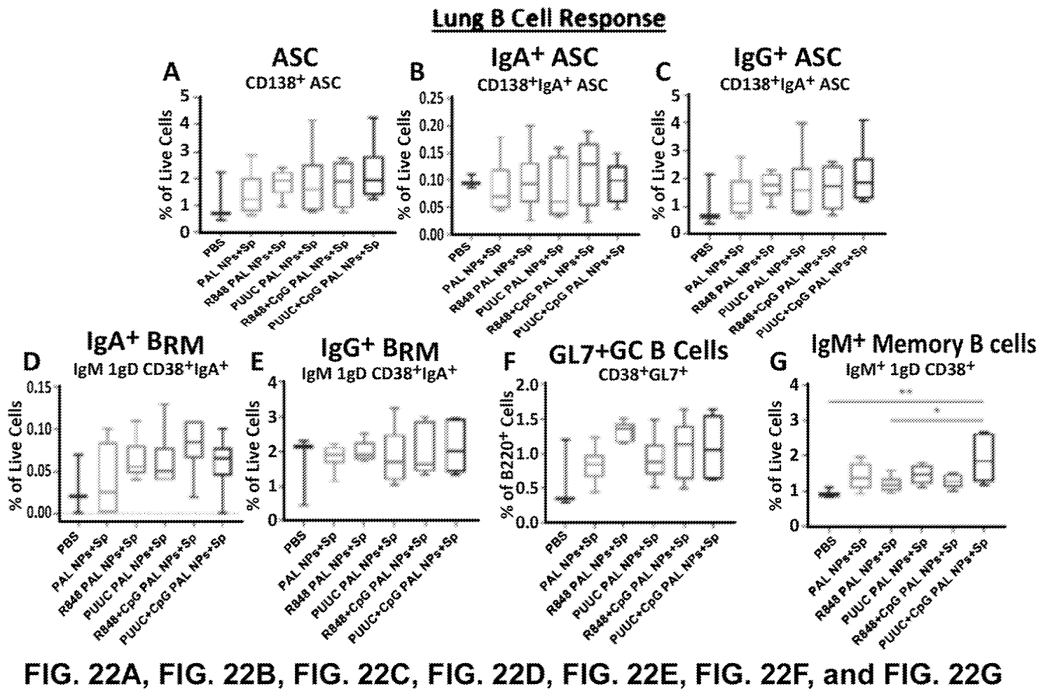

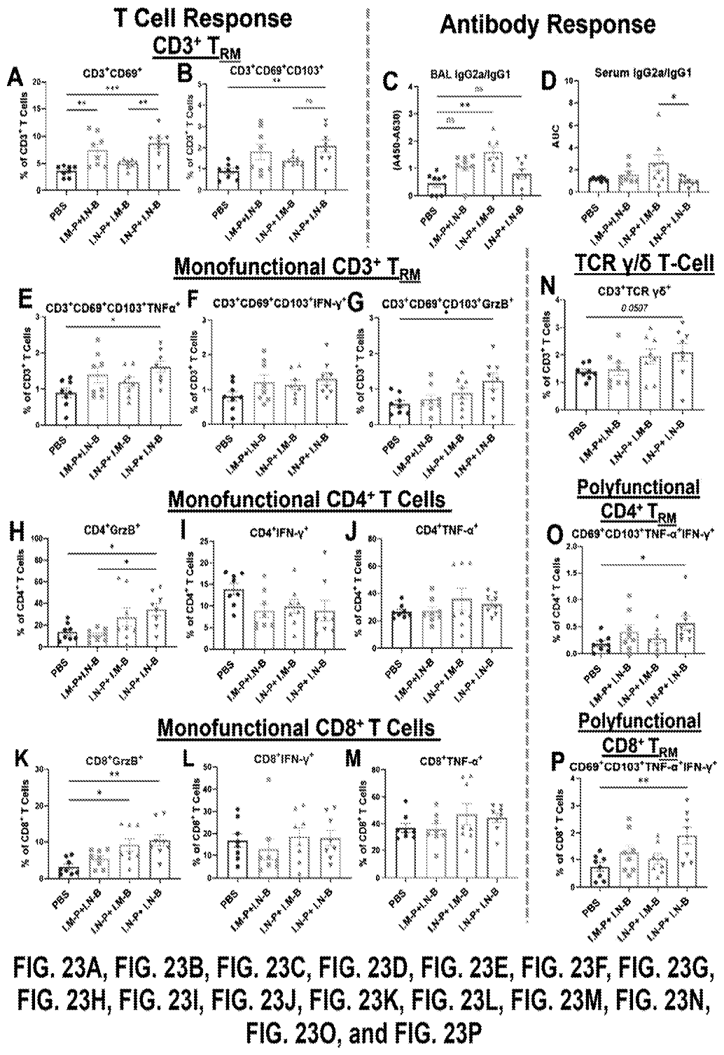

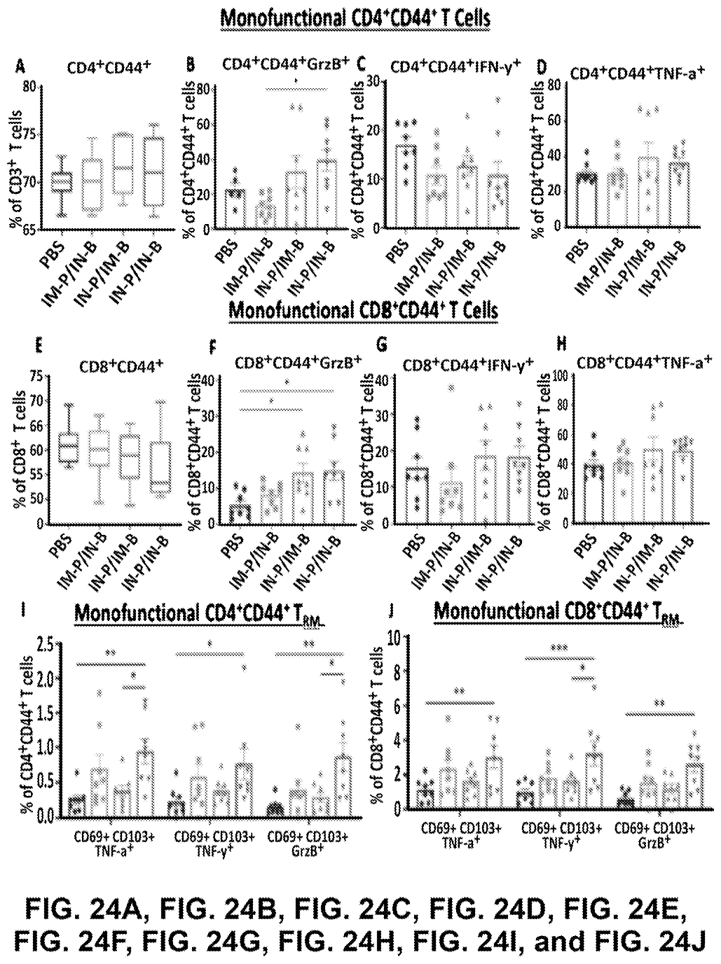

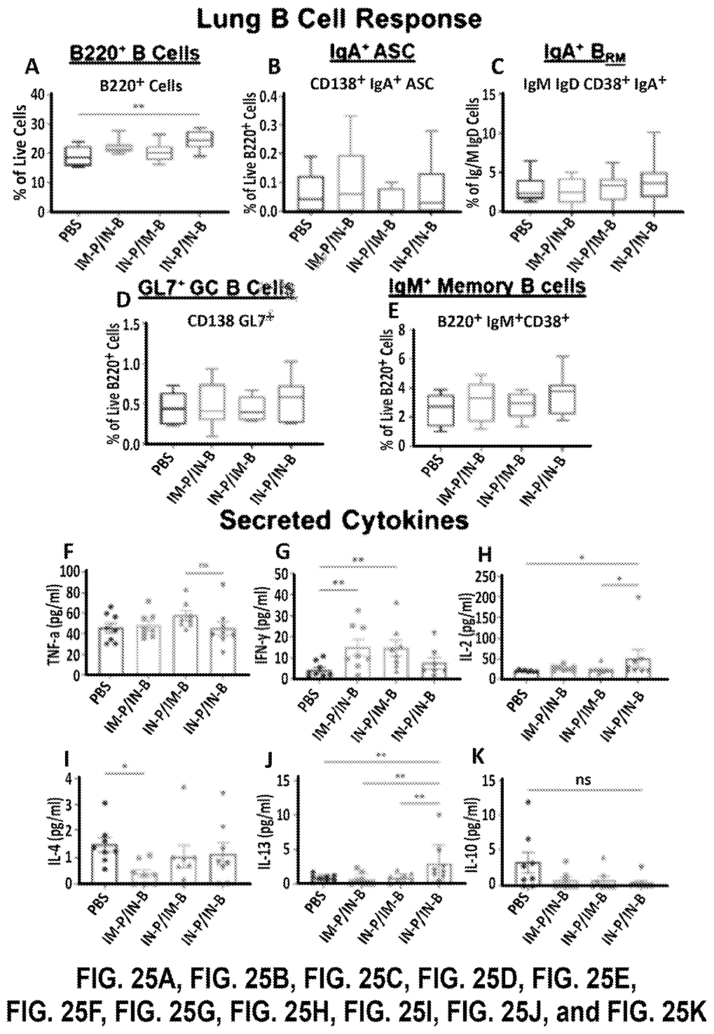

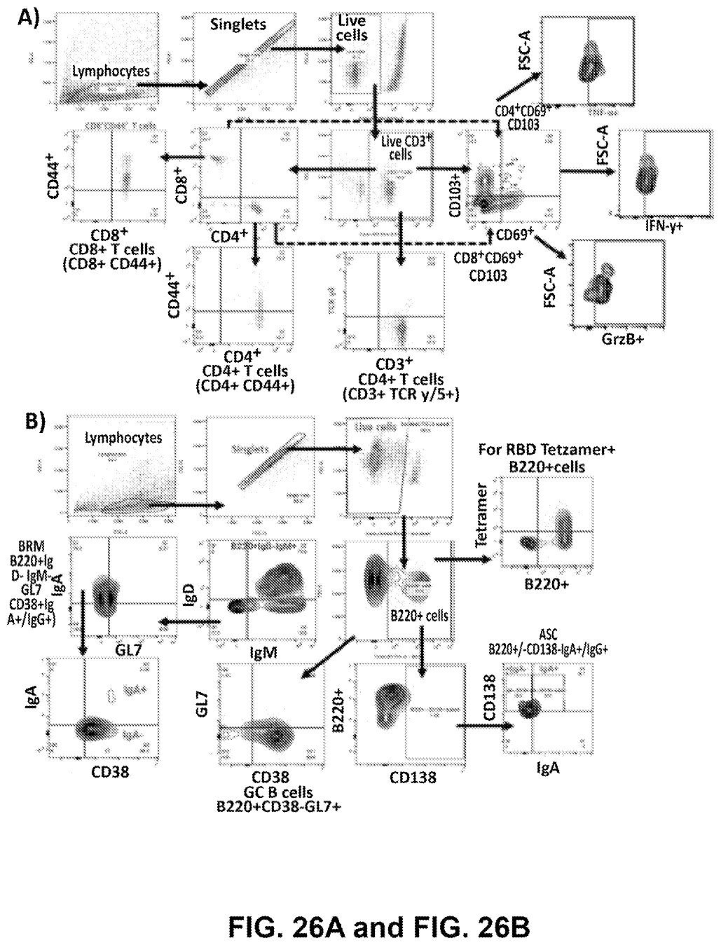

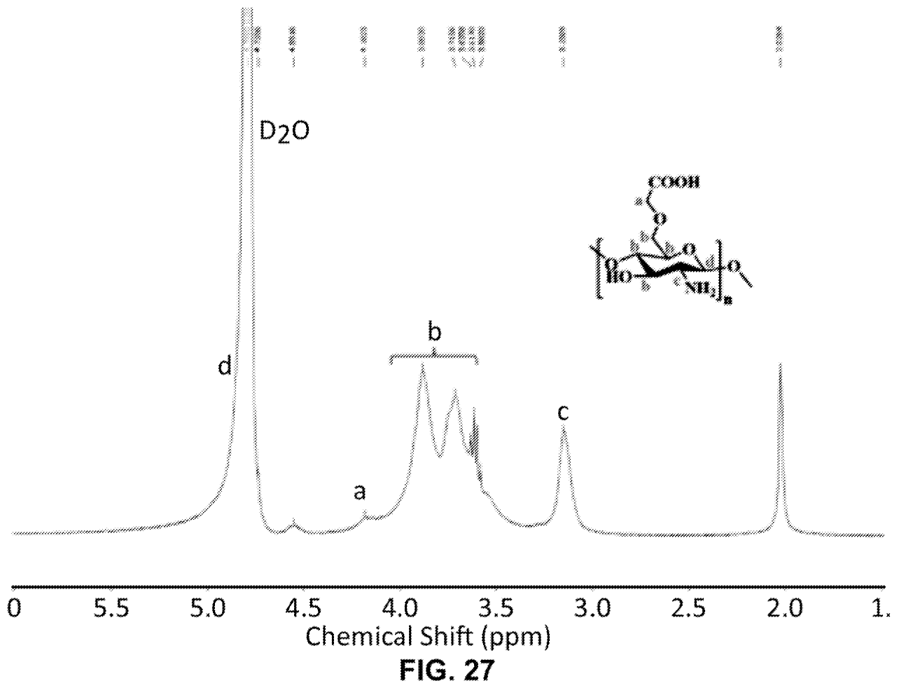

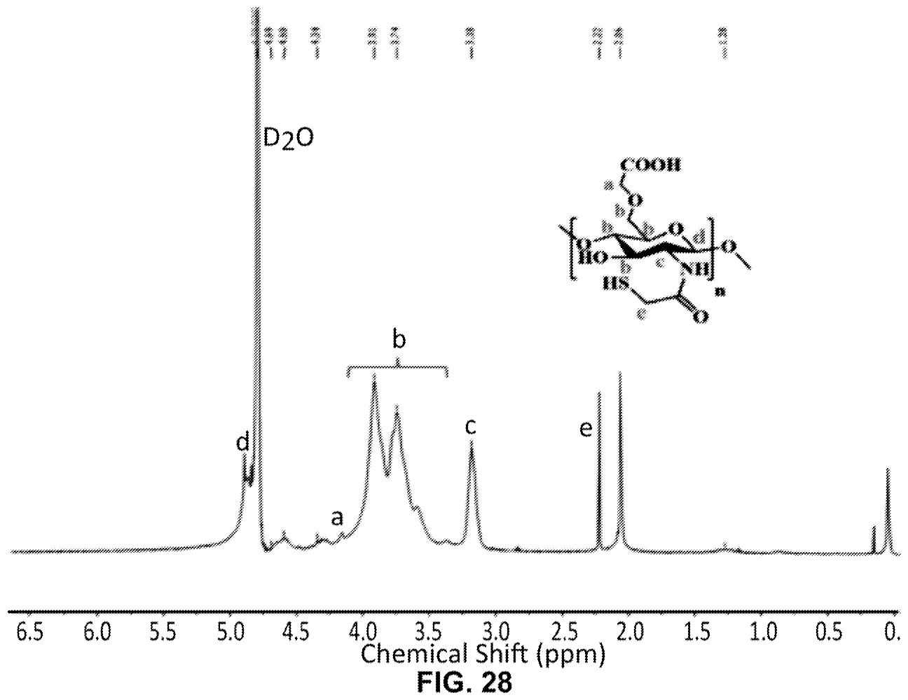

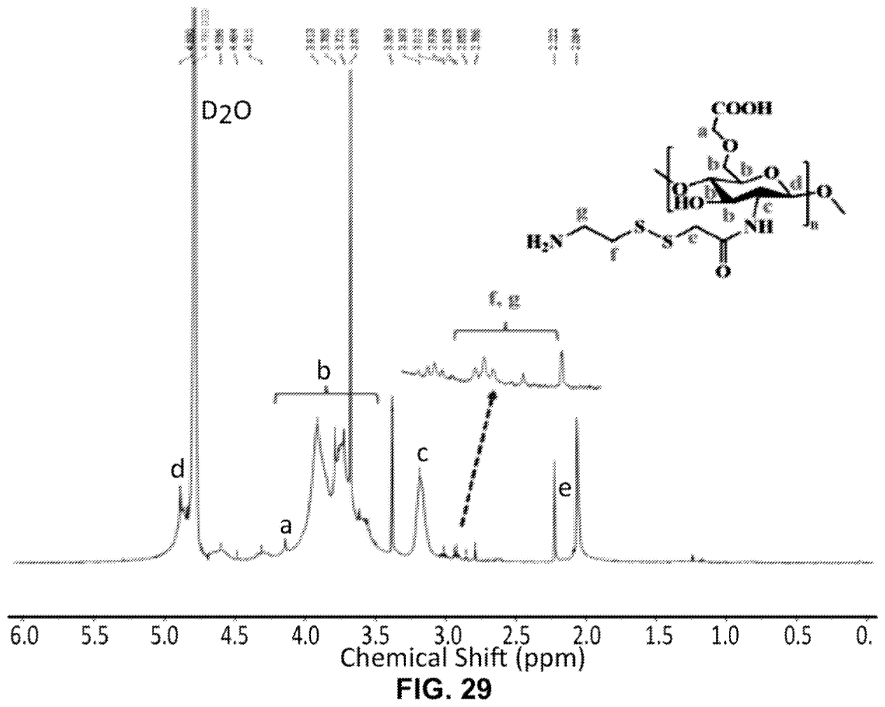

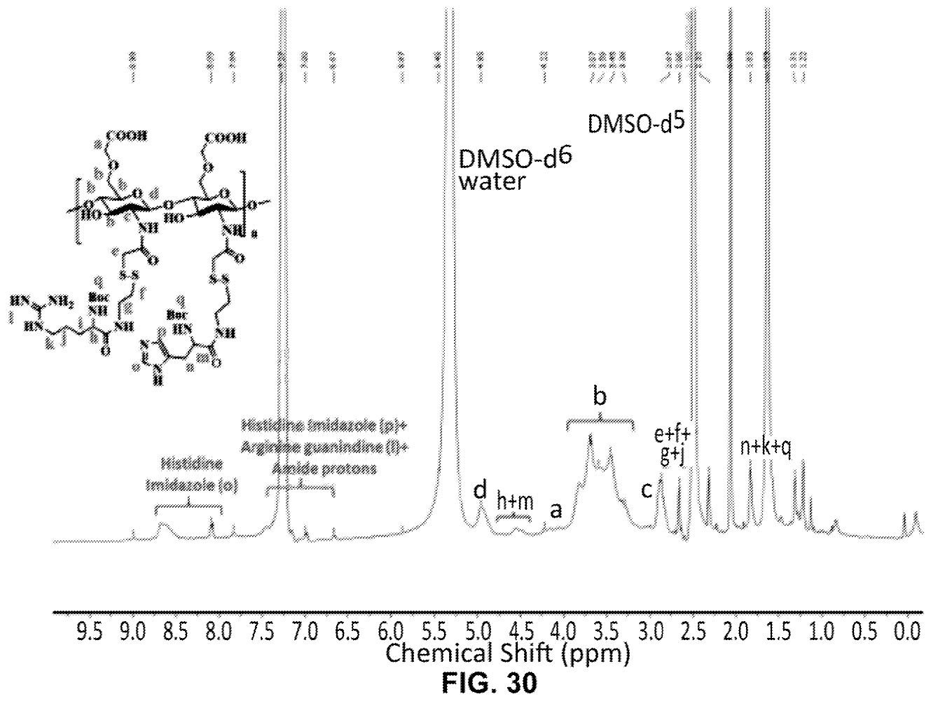

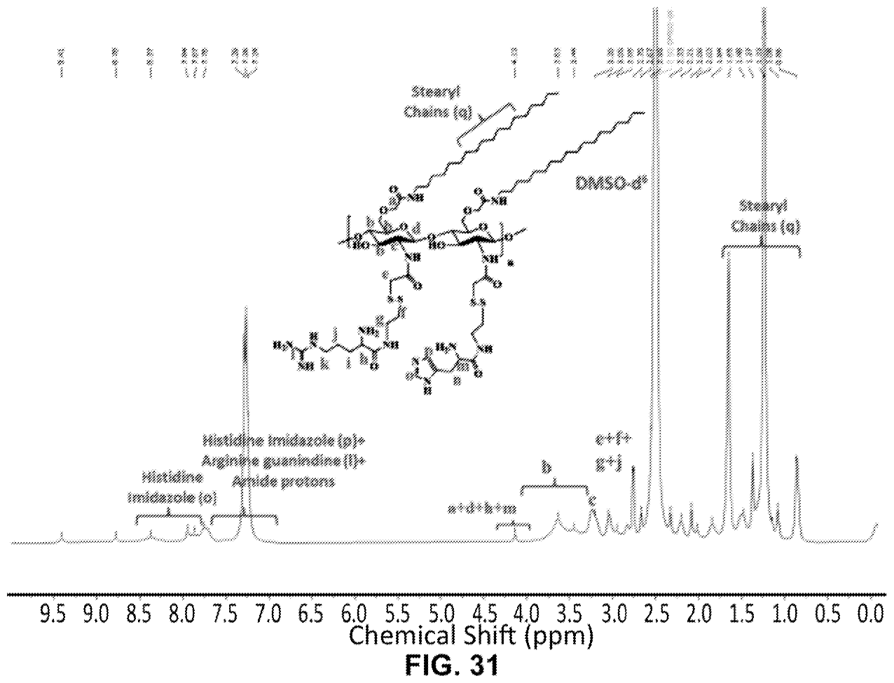

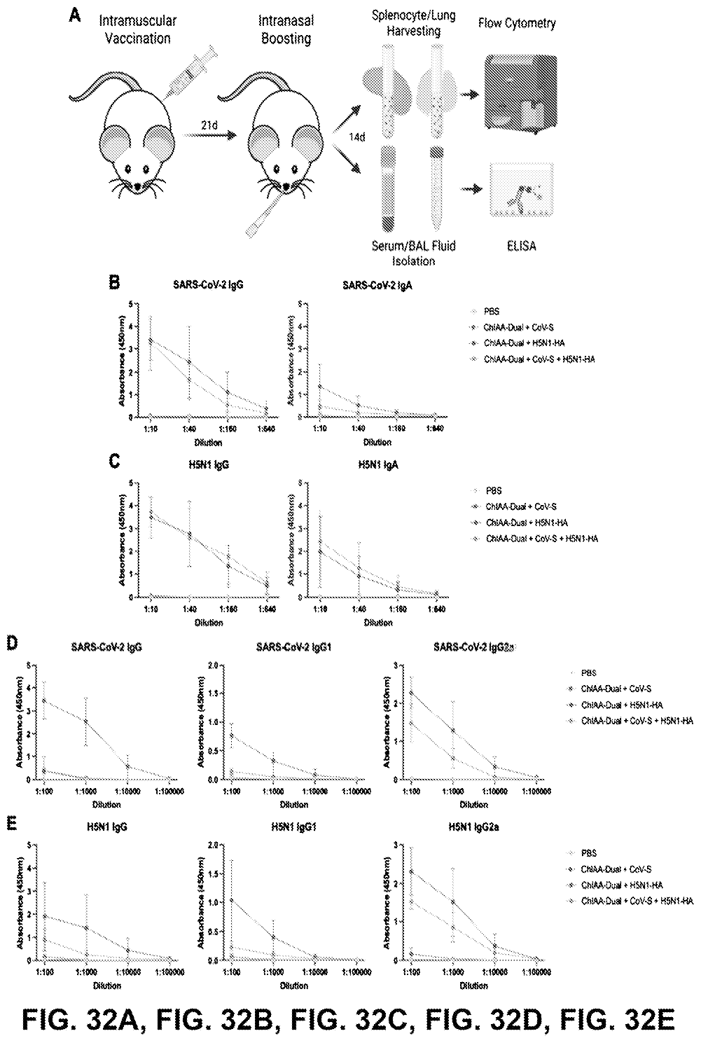

FIGS. 1 1A-11G show multi-adjuvanated CL-NP's delivered intramuscularly prime and intranasally boost with spike protein enhance B cell responses. Female BALB/c mice were immunized I.M. into both tibialis anterior muscles at day 0 (1st dose) with soluble spike protein at doses of 1000 ng with or without adjuvant-NPs (250ug) loaded with CpG, R848 and PUUC (40ug, 20ug, 20ug), respectively. On day 21, mice received the 2nd dose of protein subunit vaccines I.N with similar doses of formulations except for the CpG dose of 20ug. Mice were euthanized after two weeks on day 36 to collect blood, BAL fluid, and lungs. Plots showing the percentages of B cell subsets, including (1 IA) RBD tetramer-binding B cells, (01B) IgA+resident memory B cells (isotype switched IgA+BRM), (11C) IgM+ resident memory B cells (IgM+BRM), (11D) Germinal center B cells (GC-B cells), (11E) antibody-secreting cells (ASC), (11F) IgA+antibody antibody-secreting cells (ASC), (11G) IgG+antibody-secreting cells in lung tissues. Normality was assessed with the Kolmogorov-Smirnov test. Statistical significance was determined with the Kruskal-Wallis test and Dunn's post-hoc test for multiple comparisons. Statistical significance calculated with One-Way ANOVA and Tukey post-hoc test. *p≤0.05, **p≤0.01, ***p≤0.001, ****p5 0.0001 for all graphs. CL-NP's with the CpG and PUUC adjuvants show a non-significant increase in the RBD Tetraner+B cells and antibody-secreting cells (ASC) expressing IgG+ in lung tissues. This combination adjuvant formulation also shows a significant increase in the IgM+ resident memory B cells (IgM+BRM) and germinal center B cells (GC-B cells). While NP's with the CpG and R848 adjuvants shows a non-significant increase in 1gM+ resident memory B cells (IgM+BRM) and antibody-secreting cells (ASC)expressing IgA+.

FIGS. 12A-12G shows the synthesis and characterization of multiadjuvanated PAL-NPs. FIG. 12A shows the multistep synthetic scheme of cationic and degradable polysaccharide-amino acid-lipid (PAL) amphiphilic polymer. FIG. 128 shows a comparison of 1 H NMR spectra of amphiphilic polymer with the III NMR spectra of thiolated chitosan polymer after structural modification. FIG. 12C shows a schematic of PAL-NPs fabrication from polymer, depiction of PAL-NPs with encapsulated hydrophobic adjuvant (R848) and surface-loaded nucleic acids adjuvant (PUUC, CpG) for their delivery in both in vitro and in vivo. FIG. 12D shows physiochemical characterization of PAL-NPs: hydrodynamic diameter and zeta potential, (inset: TEM image of PAL-NPs, scale bar is 500 nm). FIGS. 12E and 12F show nanoparticle co-delivery of multi-adjuvants broadens the innate immune response in GM-CSF differentiated murine BMDCs. Murine GM-CSF differentiated BMDCs were treated with single/dual/triple adjuvanated PAL-NP formulations and controls. Analysis of cytokine level: IL-1β (E), IFN-P (12F), and IL12p70 (12G) after 24 h of adjuvanted PAL-NP treatment (n=6) from GM-CSF differentiated murine BMDCs. Error bars represent SEM (standard error of the mean). Statistical significance was determined by one-way ANOVA followed by Tukey's post-hoc test for multiple comparisons. *p≤0.05, **p≤0.01, ***p≤0.001, ****p≤0.0001 for all graphs.

FIGS. 13A-13N show a subunit nanovaccine formulation of PAL-NPs adjuvanated with RIG-1 (PUUC) and TLR9 (CpG) agonists mixed with S1 spike protein elicits robust SARS-CoV-2 mucosal and systemic humoral immunity, when delivered IM-Prime/IN-Boost. FIG. 13A show the experimental schematics: Female BALB/c mice (n=3 for PBS and n=6 for other adjuvanated PAL-NP formulations) were immunized IM into both anterior tibialis muscles at day 0 (1st dose) with vaccine formulation of adjuvanated PAL-NPs (NPs: 250 μg, PULTC: 20 μg, CpG: 40 μg and R848: 20 μg) and combined with stabilized spike (Sp) S1 trimer protein at a dose of 1000 ng respectively. On day 21, mice received the 2nd dose of vaccine formulation IN using similar doses of adjuvants, PAL-NPs, and spike protein, except for the CpG dose reduced to 20 μg. Mice were euthanized after two weeks on day 35 to collect BAL fluid and serum. BAL and serum from vaccinated mice were assayed with ELISA assay. FIG. 13B, 13C, 13D,a dn 13E show BAL fluid from vaccinated mice was assayed for anti-spike IgA (13B), IgG (13C), IgG (13D), and IgG2a (13E) with ELISA at 1:10 dilution. FIG. 13F shows calculated value of BAL IgG2a/IgG1 ratio. FIG. 13G shows anti-spike total IgG in serum at various dilutions measured by absorbance (A450-630 nm) during ELISA assays. FIG. 13H shows a comparison of area under the curve (AUC) of serum anti-spike IgG. FIG. 131 shows ACE-2 signal measured by absorbance (A450-630 nm) in spike protein neutralization assay with ELISA. Lower absorbance values indicate higher spike-neutralizing antibody levels in serum. FIG. 13J shows serum from vaccinated mice was assayed for IgG1. FIG. 13K shows a comparison of area under the curve (AUC) of serum anti-spike IgG1. FIG. 13L shows the serum from vaccinated mice was assayed for IgG2a. FIG. 13M shows a comparison of area under the curve (AUC) of serum anti-spike IgG2a. FIG. 13N shows the calculated value of serum IgG2a/IgG1 ratio. Error bars represent the SEM. Normality was assessed with the Kolmogorov-Smirnov test. Statistical significance was determined with the Kruskal-Wallis test and Dunn's post-hoc test for multiple comparisons. *p≤. 0.05, **p≤0.01, ***p≤0.001, ****p≤0.0001 for all graphs.

FIGS. 14A-14L show a subunit nanovaccine formulation of PAL-NPs adjuvanated with RIG-I (PUUC) and TLR9 (CpG) agonists, mixed with S1 spike protein elicit robust SARS-CoV-2 mucosal cellular immunity, when delivered IM-Prime/IN-Boost. FIG. 14A shows experimental schematics: Female BALB/c mice (n=3 for PBS and n=6 for adjuvanated PAL-NP formulations) were immunized IM into both anterior tibialis muscles at day 0 (1st dose) with vaccine formulation of adjuvanated PAL-NPs (NPs: 250 μg, PUUC: 20 μg, CpG: 40 μg, and R848: 20 μg) and combined with stabilized spike (Sp) S1 trimer protein at a dose of 1000 ng respectively. On day 21, mice received the 2nd dose of protein subunit vaccine formulation IN, using similar doses of adjuvants, PAL-NPs, and protein, except for the CpG dose reduced to 20 sg. Mice were euthanized on day 35 to collect lungs. Lung cells were restimulated with spike peptide pools for 6 h and stained for analysis by flow cytometry. FIGS. 14B, 14C, and 14D shows representative flow cytometry plots (FCM) (14B) and percentage of CD4+CD69+(14C) and CD4+CD69+CD103+(14D) T cell population. FIGS. 14E, 14F, and 14G show representative FCM plots (14E) and percentage of CD8+-CD69+(14F) and CD8+CD69+CD103+(14G) Tcell population. FIGS. 1411, 141, and 14J show representative FCM plots (14H) and percentage of CD4+CD44+CD69+(141) and CD4+CD44+CD69+CD103+(14J) T cell population. Lung cells were stained for B cell markers and analyzed by flow cytometry. (14K and 14L) Representative FCM plots and percentage of RBD tetramer+B220 cells. Outliers were identified by the ROUT method and removed. Error bars represent the SEM. Statistical significance was calculated using one-way ANOVA followed by Tukey's post-hoc test for the FIGS. (14C), (14D), (14F), and (141), and Bonferroni's post-hoc test for figure (14G), (14J), and (14L), for multiple comparisons. *p≤0.05, **p≤0.01, ***p≤0.001, ****p S0.0001 for all graphs. ns represents the non-significant values.

FIGS. 15A-15Q shows a subunit nanovaccine formulation of PAL-NPs adjuvanated with RIG-I (PUUC) and TLR9 (CpG) agonists, mixed with S1 spike protein elicit robust SARS-CoV-2 mucosal cellular immunity, when delivered IM-Prime/IN-Boost (15A) Experimental schematics: Female BALB/c mice (n=3 for PBS and n=6 for other formulations) were immunized IM into both anterior tibialis muscles at day 0 (1st dose) with vaccine formulation of multiadjuvanated PAL-NPs (NPs: 250 μg, PUUC: 20 μg, CpG: 40 μg, and R848: 20 μg) and combined with stabilized spike (Sp) S1 trimer protein at a dose of 1000 ng respectively. On day 21, mice received the 2nd dose of protein subunit vaccine formulation IN, using similar doses of adjuvants, PAL-NPs, and protein, except for the CpG dose reduced to 20 μg. Mice were euthanized on day 35 to collect lungs. Lung cells were restimulated with spike peptide pools for 6 h and stained for analysis by flow cytometry. FIGS. 15B and 15C shows representative flow cytometry plots (FCM) of monofunctional CD4+ TRM and percentages of cells expressing TNF-α. FIGS. 15D and 15E show representative FCM plots of monofunctional CD4+ TRM and percentages of cells expressing IFN-γ. FIGS. 15F and 15G show representative FCM plots of monofunctional CD8+ TRM and percentages of cells expressing TNF-α. FIGS. 15H and 15I show representative FCM plots of monofunctional CD8+ TRM and percentages of cells expressing IFN-γ. FIGS. 15J and 15K show representative FCM plots of monofunctional CD4+CD44+ TRM and percentages of cells expressing TNF-α. FIGS. 15L and 15M show representative FCM plots of monofunctional CD4+CD44+ TRM and percentages of cells expressing GrzB. FIGS. I5N, 15O, 15P, and ISQ show cytokine concentration in supernatants from restimulated lung cells TNF-α (15N), IFN-γ (15O), IL-1β (ISP), IL-4 (15Q). Error bars represent the SEM. Outliers were identified by the ROUT method and removed. Statistical significance was calculated using one-way ANOVA followed by Tukey's post-hoc test for FIGS. (15I) and (15K to I5Q), and Bonferroni's post-hoc test for FIGS. (15C), (15E), and (15G), for multiple comparisons. *p≤0.05, **p≤0.01, ***p≤0.001, ****p≤0.0001 for all graphs. ns represents the non-significant values.

FIGS. 16A-160 show PUUC+CpG PAL-NPs protein subunit vaccine formulation, elicit robust SARS-CoV-2 mucosal and systemic humoral immunity with IM-Prime/IN-Boost group and induces a significant level of mucosal humoral responses with IN-Prime/IN-Boost group. FIG. 16A shows experimental Schematics: Female BALB/c mice (n=8 for all groups) were immunized with three prime-boost strategies. At day 0 (1st dose), vaccine formulation of PUUC+CpG PAL-NPs (NPs: 250 μg, PUUC: 20 μg, CpG: 40 μg, and R848: 20 μg) combined with stabilized spike (Sp) S1 trimer protein (1000 ng) was administered. On day 21, mice received the 2nd dose of protein subunit vaccine formulation IN (CpG dose reduced to 20 μg). Mice were euthanized on day 35 to collect BAL fluid, and blood. BAL and serum from vaccinated mice was assayed with ELISA. FIGS. 16B to 16F show BAL fluid from vaccinated mice was assayed for anti-spike IgA (16B), IgG (16C), spike neutralization antibody (16D), IgG1 (16E), and IgG2a (16F) with ELISA at 1:5 dilution except for IgA and neutralization assay which was performed at 1:2 dilution. FIG. 16G shows anti-spike total IgG in serum at various dilutions measured by absorbance (A450-630 nm) during ELISA assay. FIG. 1611 shows a comparison of area under the curve (AUC) of serum anti-spike IgG. FIG. 161 shows ACE-2 signal measured by absorbance at 450 nm in spike protein neutralization assay with ELISA. FIG. 16J shows serum from vaccinated mice was assayed for IgG1. FIG. 16K shows a comparison of area under the curve (AUC) of serum anti-spike IgG1. FIG. 16L shows serum from vaccinated mice was assayed for IgG2a. FIG. 16M shows a comparison of area under the curve (AUC) of serum anti-spike IgG2a. FIG. 16N shows serum from vaccinated mice was assayed for IgA. FIG. 160 shows a comparison of area under the curve (AUC) of serum anti-spike IgA. Error bars represent the SEM. Normality was assessed with the Kolmogorov-Smirnov test. Statistical significance was determined with the Kruskal-Wallis test and Dunn's post-hoc test for multiple comparisons. *p≤0.05, **p≤0.01, ***p≤0.001, ****p≤0.0001 for all graphs. ns represent the not significant values.

FIGS. 17A-17M show that the PUUC+CpG PAL-NPs protein subunit vaccine formulation elicits robust SARS-CoV-2 T cell (TRM) immunity with IN-Prime/IN-Boost and B cell responses with IM-Prime/IN-Boost. FIG. 17A shows experimental schematics: Female BALB/c mice (n=8 for all groups) were immunized with three prime-boost strategies. At day 0 (1st dose), a vaccine formulation of PUUC+CpG PAL-NPs combined with stabilized spike protein (Sp) S I trimer protein was administered. On day 21, mice received the 2nd dose of protein subunit vaccine formulation IN (CpG dose reduced to 20 μg). Lung cells were restimulated with spike peptide pools for 6 h and stained for analysis by flow cytometry. FIGS. 17B to 17D show representative flow cytometry plots (FCM groups: PBS, IM-Prime/IN-Boost, and IN-Prime/IN-Boost) (17B) and percentage of CD4+CD69+(17C) and CD4+ TRM (17D) cell population. FIGS. 17E to 17G show representative FCM plots (groups: PBS, IM-Prime/IN-Boost, and IN-Prime/1N-Boost) (17E) and percentage of CD8+CD69+(17F) and CD8+ TRM (17G) cell population. Percentage of T cell populations: CD4+CD44+CD69+(17H), CD4+CD44+ TRM (17I), CD4+CD44+CD69+(17J), CD4+CD44+ TRM (17K). FIGS. 17L and 17M shows representative flow cytometry plots and percentage of RBD tetramer+B220+cells. Error bars represent the SEM. Statistical significance was calculated with One-Way ANOVA and Tukey post-hoc test for multiple comparisons. *p≤0.05, **p≤0,01, ***p≤0.001, ****p≤0.0001 for all graphs. ns represents the not significant values.

FIGS. 18A-18M show PUUC+CpG PAL-NPs subunit vaccine formulation enhances TH1 type immunity with IN-Prime/IN-Boost group. FIG. 18A shows experimental schematics: Female BALB/c mice (n=8 for all groups) were immunized with three prime-boost strategies. At day 0 (1st dose), a vaccine formulation of PUUC+CpG PAL-NPs combined with a stabilized spike (Sp) Si trimer protein was administered. On day 21, mice received the 2nd dose of protein subunit vaccine formulation IN (CpG dose reduced to 20 μg). Mice were euthanized on day 35 to collect lungs. Lung cells were restimulated with spike peptide pools for 6 h and stained for analysis by flow cytometry. (18B, 18C, and 18D) Representative FCM plots (groups: PBS, IM-Prime/IN-Boost, and IN-Prime/:N-Boost) which monofunctional CD4+ TRM cells expressing TNF-α (18B), IFN-γ (18C), and GrzB (18D). FIG. 18E shows the percentages of monofunctional CD4+ TRM cells expressing TNF-α, IFN-γ, and GrzB. (18F to 18H) Representative FCM plots (groups: PBS, IM-Prime/IN-Boost, and IN-Prime/IN-Boost) of monofunctional CD8+ TRM cells expressing TNF-α (F), IFN-γ (18G), and GrzB (18H). FIG. 181 shows the percentages of monofunctional CD8+ TRM cells expressing TNF-α, IFN-γ, and GrzB. FIG. 18J shows the percentages of polyfunctional CD4+ TRM cells co-expressing TNF-α and GrzB. FIG. 18K shows the percentages of polyfunctional CD4+ TRM cells co-expressing IFN-γ and GrzB. FIG. 18L shows the percentages of polyfunctional CD8+ TRM cells co-expressing TNF-α and GrzB. FIG. 18M shows the percentages of polyfunctional CD8+ TRM cells co-expressing IFN-γ and GrzB. Error bars represent the SEM. Statistical significance for cytokine+ T cell frequencies was calculated with One-Way ANOVA and Tukey post-hoc test for multiple comparisons. *p≤0.05, **p≤0.01, ***p≤0.001, ****p≤0.0001 for all graphs, ns represent the not significant values.

FIGS. 19A, 19B, and 19C shows the synthetic scheme of PAL polymer and PAL-NPs characterization. FIG. 19A shows multistep synthesis of polysaccharide-amino acid-lipid amphiphilic (PAL) polymer- (i) Chitosan, NaOH, −10° C. incubation, 1 h, Cl—CH2—COOH, heat (45° C.), 24 h (ii) EDC/NHS, HS-CH2COOH (iii) NH2—CH2—CH2—SH, cysteamine, pH=6 (iv) EDC/NHS, Nu-Boc-L-arginine and Na-Boc-L-histidine (v) EDC/NHS, CH3(CH2)17NH2, heating 80° C. (vi) TFA/4M HCl in Dioxane, Boc deprotection. FIG. 19B shows the sstimation of thiols and disulfide concentration in thiolated polymer and cysteamine conjugated chitosan polymer by Elmann assay. FIG. 19C shows time-dependent degradation study of the PAL-NPs by DLS analysis in the presence of DTT (10 mM).

FIGS. 20A-20M show PUUC+-CpG PAL-NP protein subunit vaccine formulation with S1 spike protein, elicits robust SARS-CoV-2 elicits T cell immunity when delivered IM-Prime/IN-Boost. On days 0 (IM prime) and 21 (IN boost), female BALB/c mice were immunized with adjuvanated PAL-NP vaccine formulation with S1 spike protein (see Materials/Methods and Table 1 for doses). Mice were euthanized, and lungs were collected on Day 35 (one-week post-boost). Lung cells were restimulated with spike peptide for 6 h. FIGS. 20A and 20B shows the percentage of CD3+CD69+ and CD3+CD69+CD103+(CD3+ TRM) cell population. FIGS. 20C, 20D, and 20E shows the percentage of monofunctional CD3+TRM cells expressing TNF-α, IFN-γ, and GrzB. FIGS. 20F, 20G, and 20H show the percentage of monofunctional CD4+ T cells expressing TNF-α, IFN-1, and GrzB. FIG. 20I shows the percentage of Monofunctional CD4+ TRM cells expressing GrzB. FIGS. 20J, 20K and 20L show the percentage of monofunctional CD8+ T cells expressing TNF-α, IFN-γ, and GrzB. FIG. 20M shows the percentage of polyfunctional CD8+ TRM cells expressing GrzB. Error bars represent the SEM. Statistical significance was calculated using one-way ANOVA followed by Tukey's post-hoc test for the FIGS. (20A), (20B), (20J), and (20K), and Bonferroni's post-hoc test for the FIGS. (20C), (20I), and (20M), for multiple comparisons. *p 5 0.05, **p≤0.01, ***p≤0.001, ****p S0.0001 for all graphs.

FIGS. 21A-21L show PUUC+CpG PAL-NP protein subunit vaccine formulation with Si spike protein, elicits robust SARS-CoV-2 elicits T cell immunity when delivered IM-Prime/IN-Boost. On days 0 (IM prime) and 21 (IN boost), female BALBic mice (n=3 for PBS and n=6 for other adjuvanated PAL-NP groups) were immunized with adjuvanated PAL-NP vaccine formulation with S I spike protein (see Materials/Methods and Table I for doses). Mice were euthanized, and lungs were collected on Day 35 (one-week post-boost). Lung cells were restimulated with spike peptide for 6 h. FIG. 21A shows the percentage of CD4+CD44+ cell population. FIGS. 21B, 21C, and 21D show the percentage of CD4+CD44+ cells expressing TNF-α, IFN-γ, and GrzB. FIG. 21E shows the percentage of monofunctional cells expressing CD8+CD44+. FIGS. 21F, 21G, and 21H shows the percentage of monofunctional CD8+CD44+ T cells expressing TNF-α, IFN-γ, and GrzB. FIG. 21I shows the percentages of monofunctional CD4+ TRM cell population co-expressing both TNF-α and IFN-γ. (21J) Percentage of monofunctional CD8+ TRM cell population co-expressing both TNF-α and IFN-γ. (21K) Percentage of CD3+CD4-CD8- cell population. (21L) IL-13 Cytokine concentration in supernatants from restimulated lung cells. Error bars represent the SEM. Statistical significance was calculated using one-way ANOVA followed by Tukey's post-hoc test for the FIGS. (B), (21F), and (21H), and Bonferroni's post-hoc test for the FIGS. (21D), (21I), and (21K), for multiple comparisons. Statistical significance for cytokine concentrations was calculated with One-Way ANOVA and Tukey post-hoc test. *p≤0.05, **p≤0.01, ***p≤0.001, ****p≤0.0001 for all graphs.

FIGS. 22A-22G shows the analysis of lung B cell responses when multiple adjuvanated PAL-NP protein subunit vaccine formulations are delivered to mice via IM-Prime/IN-Boost vaccination. On days 0 (IM prime) and 2I (IN boost), female BALB/c mice (n=3 for PBS and n=6 for other PAL-NP groups) were immunized with adjuvanated PAL-NP vaccine formulation with S1 spike protein (see Materials/Methods and Table 1 for doses). Mice were euthanized, and lungs were collected on Day 35 (one-week post-boost). FIG. 22A shows the percentage of CD138+ASC population. FIG. 22B shows the percentage of IgA+ASC population. FIG. 22C shows the percentage of IgG+ASC population. FIG. 22D shows the percentage of IgA+BRM cell population. FIG. 22E shows the percentage of IgG+BRM cell population. FIG. 22F shows the percentage of GL7+GC B cell population. FIG. 22G shows the percentage of IgM+Memory B cell population. Error bars represent the SEM. Statistical significance was calculated with One-Way ANOVA and Tukey post-hoc test. *p≤0.05, **p≤0.01, ***p≤0.001, ****p≤0.0001 for all graphs.

FIGS. 23A-23P show the PUUC+CpG PAL-NP protein subunit vaccine formulation with S1 spike protein, elicits robust SARS-CoV-2 lung-specific T cell immune response with IN-Prime/IN-Boost strategy. Female BALB/c mice were immunized with PUUC+CpG PAL-NP vaccine formulation with S I spike protein (see Materials/Methods and Table I for doses). Female BALB/c mice (n=8 for all groups) were immunized with three prime-boost strategies: IM-Prime/IN-Boost, IN-Prime/IM-Boost, and IN-Prime/IN-Boost. Mice were euthanized, and lungs were collected on Day 35. Lung cells were restimulated with spike peptide for 6h. FIG. 23A shows the percentage of CD3+CD69+ cell population and, (23B) percentage of CD3+CD69+CD103+(CD3+ TRM) cell population. FIG. 23C shows the calculated value of BAL IgG2a/IgG1. FIG. 23D shows the calculated value of BAL IgG2a/IgG1. FIGS. 23E, 23F, and 23G show the percentages of monofunctional CD3+ TRM cells expressing TNFα, IFNγ, and GrzB. FIGS. 23H, 23I, and 23J show the percentages of monofunctional CD4+Tcells expressing TNFα, IFNγ, and GrzB. FIGS. 23K, 23L, and 23M show the percentages of monofunctional CD8+ T cells expressing TNFα, IFNγ, and GrzB. FIG. 23N shows the percentages of CD3+ TCR γϵ cells. FIG. 230 shows the percentages of polyfunctional CD8+ TRM cells co-expressing TNF-α and IFN-γ. FIG. 23P shows the percentages of polyfunctional CD8+ TRM cells co-expressing TNF-α and IFN-γ. Error bars represent the SEM. Statistical significance was calculated with One-Way ANOVA and Tukey post-hoc test. *p≤0.05. **p≤0.01, ***p≤0.001, ****p≤0.0001 for all graphs. Ns represent the non-significant values.

FIGS. 24A-24J show PUUC+CpG PAL-NP protein subunit vaccine formulation with S1 spike protein, elicits robust SARS-CoV-2 T cell immune responses with IN-Prime/IN-Boost route. Female BALB/c mice were immunized with PUUC+CpG PAL-NP vaccine formulation with Si spike protein (see Materials/Methods and Table 1 for doses). Female BALB/c mice (n=8 for all groups) were immunized with three prime-boost strategies: IM-Prime/IN-Boost, IN-Prime/IM-Boost, and IN-Prime/IN-Boost. Mice were euthanized, and lungs were collected on Day 35. Lung cells were restimulated with spike peptide for 6 h. FIG. 24A shows the percentages of CD4+CD44+ cell population. FIGS. 25B, 24C, and 24D show the percentages of monofunctional CD4+CD44+ cells expressing GrzB, IFN-γ, and TNFα. FIG. 24E shows the percentages of monofunctional cells expressing CD8+CD44+. FIGS. 24F, 24G, and 24H shows the percentages of monofunctional CD8+CD44+ T cells expressing GrzB, IFN-γ, and TNFα. FIG. 241 shows the percentages of monofunctional CD4+CD44+ TRM cells expressing TNFα, IFN-γ, and GrzB. FIG. 24J shows the percentages of monofunctional CD8+CD44+ TRM cells expressing TNFα, IFN-γ, and GrzB. Error bars represent the SEM. Statistical significance T cell frequencies were calculated with One-Way ANOVA and Tukey post-hoc test. *p≤0.05, **p 0.01, ***p≤0.001, ****p≤0.0001 for all graphs.

FIGS. 25A-25K show lung-specific B cell and T cell (secreted cytokine) responses, when PUUC+CpG PAL-NP protein subunit vaccine formulation and mixed with S I spike protein, delivered with three different prime-boost routes. Female BALB/c mice were immunized with PUUC+CpG PAL-NP vaccine formulation with S1 spike protein (see Materials/Methods and Table I for doses). Female BALB/c mice (n=8 for all groups) were immunized with three prime-boost strategies: IM-Prime/IN-Boost, IN-Prime/IM-Boost, and IN-Prime/IN-Boost. On days 0 (prime) and 21 (boost), mice were euthanized, and lungs were collected on Day 35. Quantification of B cell response (25A) Percentage of B220+B cell population. FIG. 25B shows the percentage of IgA+ASC cell population. (FIG. 25C shows the percentage of IgA+BRM cell population. FIG. 25D shows the percentage of GL7+GC B cell population. FIG. 25E shows the percentage of IgM+Memory B cell population. Lung cells were restimulated with spike peptide for 6h. FIGS. 25F to 25K show the cytokine concentration in supernatants from restimulated lung cells: TNFα, IFN-γ, IL-2, IL-4, IL-13, and IL-10. Error bars represent the SEM. Statistical significance T cell frequencies was calculated with One-Way ANOVA and Tukey post-hoc test. Statistical significance for cytokine concentrations was calculated with one-Way ANOVA and Tukey post-hoc test *p≤0.05, **p≤0.01, ***p≤0.001, ****p s 0.0001 for all graphs. ns represents the non-significant values.

FIGS. 26A and 26B show gating strategies for analysis of adaptive immune responses in the lungs. FIG. 26A shows gating strategies to identify CI)4+ and CD8+ T cells and gating strategies to identify cytokine producing CD4+ and CD8+ T cells. FIG. 26B shows gating strategies to identify antigen-specific and polyclonal B cells.

FIG. 27 shows 400 MHz 1 H NMR spectrum of the carboxylated chitosan (OCMC) in D2O with 1% DCI.

FIG. 28 shows 400 MHz 1H NMR spectrum of the thiolated OCMC in D2O with 1% DC1.

FIG. 29 shows 400 MHz 1 H NMR spectrum of the OCMC-S-S-Cys in D2O with 1% DC1.

FIG. 30 shows 400 MHz 1H NMR spectrum of the OCMC-S-S-(A/H) in DMSO-d6.

FIG. 31 shows 400 MHz 1H NMR spectrum of the OCMC-S-S-(A/H)-SA in DMSO-d6.

FIGS. 32A-32E show that chitosan-IAA nanoparticle systems induce strong joint antibody responses in vivo. FIG. 32A shows a schematic of in vivo experiment for assessing antibody titers and T cell populations post-vaccination with Chitosan-IAA-TPP adjuvant-nanoparticles and SARS-CoV-2 S protein and/or H5N I HA protein. Absorbance data derived from ELISAs conducted on BAL fluid (FIG. 32B, 32C) and serum from vaccinated mice (FIG. 321), 32E).

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups. etc. of these materials are disclosed that while specific reference of each various individual and collective combinations and permutation of these compounds may not be explicitly disclosed, each is specifically contemplated and described herein. For example, if a particular nanoparticle is disclosed and discussed and a number of modifications that can be made to a number of molecules including the nanoparticle are discussed, specifically contemplated is each and every combination and permutation of nanoparticle and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect, disclosed herein are nanoparticles comprising a polymer having an outer surface and an inner core a lipid, and an amino acid. A nanoparticle is a small particle that can range from between I to 100 nm in size. Nanoparticles can exhibit notably different physical and chemical properties in comparison to their larger material counterparts. Nanoparticles can be created naturally, for examples as by-products of combustion reactions, or produced purposefully through engineering to perform a specialized function. The use of nanoparticles spans across a wide variety of industries, from healthcare and cosmetics to environmental preservation and air purification.

In healthcare field, nanoparticles can be used in a variety of ways, one of which is for delivery of substances such as antibodies, drugs, imaging agents, and other substances to certain parts of the body. For example, nanoparticles can be used in detection, diagnosis, prevention, and treatment of healthcare issues in patients. In some examples, the substance or substances of interest can be loaded into the core of the nanoparticle, loaded onto the surface of the nanoparticle, or both.

Nanoparticles can be from 50 nm to 600 nm, 100 nm to 400 m, 150 to 300 nm, or 200-250 nm. For example the nanoparticle can be 50, 55, 60, 65, 70, 75, 80, 85, 90, 95, 100, 105, 110,115, 120,125, 130,135, 140,145, 150, 155, 160,165, 170, 175, 180, 185, 190, 195, 200, 205, 210, 215, 220, 225, 230, 235, 240, 245, 250, 255, 260, 265, 270, 275, 280, 290, 300, 325, 350, 375, 400, 425, 450, 475, or 500 nm.

In one aspect, the disclosed nanoparticles comprise polymers. Polymers are macromolecules formed by the chemical bonding of large numbers of smaller molecules, or repeating units, called monomers. The number of monomers within the polymer molecule can vary greatly, and the degree to which regularity appears in the order, relative orientation, and the presence of differing monomers within the same polymer molecule can vary as well. In many synthetic and some natural polymers, the number of monomers (sometimes referred to as the degree of polymerization) can be determined exactly, often in order to tailor the properties of the material.

Monomers bonded together in twos, threes, and fours are called dimers, timers, and tetramers, respectively, and these short repeating units are further called oligomers. There are numerous possible combinations of monomers that can combine to form a polymer. The simplest form of polymer is one that is made up of only one type of monomer (homopolymer). Copolymers are composed of monomers that differ from one another. The degree to which they differ, either by structure or composition, and the quantities of each type of monomer relative to one another in the same polymer molecule can impact that material's chemical and physical properties.

The term “polysaccharide” refers to a polymeric carbohydrate molecule composed of a number of monosaccharide units that are covalently linked together by glycosidic linkages. Hydrolysis of the glycosidic linkages in a polysaccharide by chemical or biochemical (e.g., enzymatic digestion) reactions can produce the constituent monosaccharides or oligosaccharides. Monosaccharides are simple sugar molecules, including molecules with a chemical formula of Cₓ(H₂O)_{y}, wherein in x and y are integers that are typically at least about 3 and no more than about 10, as well as modified molecules thereof, such as amino sugars (e.g., galactosamine, glucosamine, N-acetylglucosamine). Oligosaccharides are polymers containing a small number (e.g., about 3 to about 9) of monosaccharides. As used herein, “polysaccharide” may refer to a naturally occurring full length polysaccharide molecule, a mixture of any combinations of hydrolysis products (including monosaccharide, oligosaccharide and polysaccharide species) of a full length polysaccharide molecule, any chemically modified or functionalized derivative of the full-length polysaccharide molecule or its hydrolysis product. or any combinations thereof. The polysaccharide may be linear or branched, a single chemical species or a mixture of related chemical species (such as molecules with the same basic monosaccharide units, but different number of repeats). As used herein biocompatible polymers include, but are not limited to polysaccharides such as alginate, Fungal Pullulan, Scleroglucan, Chitin, Chitosan, Elsinan, Bacterial Xanthan gum, Curdlan, Dextran, Gelatin, Levan, Emulsan, Cellulose, Hyaluronic Acid hydrophilic polypeptides; proteins such as collagen, fibrin, and gelatin; poly(amino acids) such as poly-L-glutamic acid (PGS), gamma-polyglutamic acid, poly-L-aspartic acid, poly-L- serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof Biocompatible polymers can also include polyamides, polycarbonates, polyalkylenes, polyalkylene glycols, polyalkylene oxides, polyalkylene terepthalates, polyvinyl alcohols (PVA), methacrylate PVA(m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpyrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerate), polyanhydrides, poly(acrylic acids), polyglycolides, poly(urethanes), polycarbonates, polyphosphate esters, polyphospliazenes, derivatives thereof, linear and branched copolymers and block copolymers (including triblock copolymers) thereof, and blends thereof,

In some embodiments the particle contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95. 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like.

In one aspect, disclosed herein are nanoparticles comprising a polymer having an outer surface and an inner core, wherein the polymer comprises a polysaccharide (such as, for example chitosan), a lipid, and an amino acid.

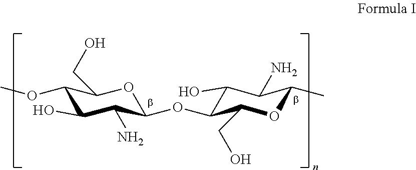

Chitosan is a natural polycationic linear polysaccharide derived from partial deacetylation of chitin. Chitin is the structural element in the exoskeleton of insects, crustaceans, and cell walls of fungi. Chitosan is made of β-(1-4)-linked D-glucosamine and N-acetyl-D-glucosamine randomly distributed within the polymer. Chitosan can be used in various applications due to its biocompatibility, non-toxicity, low allergenicity and biodegradability. The degree of deacetylation and the molecular weight of chitosan can impact the biological properties of chitosan. Chitosan is made from the deacetylation of chitin. Chitosan has the following formula:

As noted above, the disclosed nanoparticles can comprise one or more amino acids. The term “amino acid” refers to naturally occurring and synthetic α, β, γ, or δ amino acids, and includes but is not limited to, amino acids found in proteins, such as glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In certain embodiments, the amino acid is in the L-configuration. In certain embodiments, the amino acid is in the D-configuration. In one aspect, disclosed herein are nanoparticles comprising a polymer having an outer surface and an inner core, wherein the polymer comprises a polysaccharide (such as, for example chitosan), a lipid, and an amino acid (including but not limited to arginine at 0% to 100% by weight of total amino acids and/or histidine at 0% to 100% by weight of total amino acids).

Arginine (2-amino-5-guanidinovaleric acid)) is an amino acid coded for as part of ribosomal protein synthesis in humans. Arginine has the following formula:

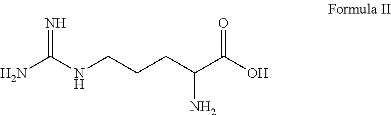 [SMILES: N=C(N)NCCCC(N)C(=O)O]

Biologically available arginine comes from three sources: (1) recycling of amino acids from normal cellular protein turnover, (2) dietary intake, and (3) de novo synthesis from arginine precursor compounds. The human body expresses enzymes that are able to synthesize arginine endogenously, and therefore it is not an essential amino acid that needs to be obtained from a person's diet. The majority of arginine for host metabolic requirements in non-stressed states is obtained endogenously, mostly, from protein turnover.

Histidine (a-amino-b-[4-imaidazole]-propionic acid is an amino acid used by the body in growth, to repair damaged tissues, and make blood cells. Further, is helps protect nerve cells and is used by the body to make histamine. Histidine has the following formula:

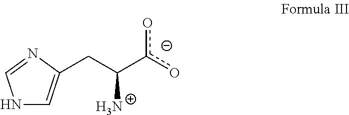

Histidine is not synthesized de novo in humans, but rather requires that humans and other animals ingest histidine or histidine-containing proteins. Sources of histidine include grain products and milk and dairy products.

As noted above, the disclosed nanoparticles can comprise one or more lipids. Lipids include fatty, waxy, or oily compounds that are soluble in organic solvents and insoluble in polar solvents such as water. Lipids can include fats and oils, like triglycerides, phospholipids, waxes, or steroids. Lipids can be made of a glycerol backbone, 2 fatty acid tails that are hydrophobic, and a phosphate group that is hydrophilic.

Fats and oils are esters made up of glycerol (a 3-carbon sugar alcohol/polyol) and 3 fatty acids. Fatty acids are hydrocarbon chains of differing lengths with various degrees of saturation that end with carboxylic acid groups. Additionally, fatty acid double bonds can either be cis or trans, creating many different types of fatty acids. Fatty acids in biological systems usually contain an even number of carbon atoms and are typically 14 carbons to 24 carbons long. Triglycerides store energy, provide insulation to cells, and aid in the absorption of fat-soluble vitamins. Fats are normally solid at room temperature, while oils are generally liquid.

Another type of lipid is wax. Waxes are esters made of long-chain alcohol and a fatty acid.

A further class includes steroids, which have a structure of 4 fused rings. One important type of steroid is cholesterol. Cholesterol is produced in the liver and is the forerunner to many other steroid hormones, such as estrogen, testosterone, and cortisol. It is also a part of cell membranes, inserting itself into the bilayer and influencing the membrane's fluidity. Conjugated to form the disclosed nanoparticles the lipids and amino acids can be conjugated to the polymer. As used herein, “conjugated” refers to polymers in which a backbone of alternative single and multiple bonds result in a-conjugation by overlap of the it-orbitals, giving rise to a continuum of energy states called a band structure. The overlapping i-orbitals can create a system of delocalized π-electrons, which can result in certain optical and electronic properties. Conjugated polymers include polythiophene, polyaniline, polypyrrole, polyphenylene, polyphenylene-ethynylene, polyacetylene, and polydiacetylene. For example, the amino acid can be conjugated to the C-2 carbon in chitosan and the lipid can be conjugated to a C-6 carbon in chitosan.

A disulfide link, or disulfide bond, is a covalent bond between two sulfur atoms (-S-S—) formed by the coupling of two thiol (—SH) groups. Herein, the disulfide link can link the lipid-chitosan to the arginine and/or histidine to make the copolymer present in the nanoparticle. A disulfide link can be synthesized by using cysteamine.

In one aspect, the nanoparticles disclosed herein can comprise an outer surface of the nanoparticle is hydrophilic and/or wherein the inner core of the nanoparticle is hydrophobic.

Hydrophilic refers to a surface that has a strong affinity for water and aqueous solutions. Hydrophilic surfaces have a high surface energy, attract water, and allow wetting of the surface. They can have a droplet contact angle measurement of less than 90 degrees.

Hydrophobic refers to a surface that has a low affinity for water and aqueous solutions. A hydrophobic surface is water repelling, has low surface energy, and resists wetting.

The nanoparticles are functionalized by being loaded with agents (including, but not limited to a therapeutic agent) on the outer surface and/or the inner core. In some aspects, wherein the outer surface of the nanoparticle is loaded with a first agent (such as, for example, a nucleic acid, a polynucleotide, peptide, protein, antibody, a siRNA molecule, a miRNA molecule, a shRNA molecule, a pDNA molecule, viral particle, heat killed virus, inactivated virus, or any combination thereof) and/or wherein the inner core of the nanoparticle is loaded with a second agent (such as, for example, a small molecule, immune adjuvants, fluorochrome, contrast agents including, but not limited to hydrophobic agents). The agent loaded on the outer surface can be hydrophilic and the agent loaded in the inner core can be hydrophobic.

Zeta potential is a physical property exhibited by any particle in suspension, macromolecule, or material surface. It can be used to optimize the formulations of suspensions, emulsions, and protein solutions, predict interactions with surfaces, and optimize the formation of films and coatings. Factors that affect zeta potential include pH, conductivity, and concentration of a formulation component. In some aspects, the nanoparticle has a zeta potential from +10 mV to +90 mV, +20 mV to +60 mV, +30 mV to +37 mV including, but not limited to +10, 11, 12, 13, 14, 15, 16, 17, 18, 19, 20, 21, 22, 23, 24, 25, 26, 27, 28, 29, 30, 30.1, 30.2, 30.3, 30.4, 30.5, 30.6, 30.7, 30.8, 30.9, 31.0, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 mV.

Many adjuvants contain a substance designed to protect the antigen from rapid catabolism, such as aluminum hydroxide or mineral oil, and a stimulator of immune responses, such as lipid A, Bordetella pertussis or Mycobacterium tuberculosis derived proteins. Certain adjuvants are commercially available as, for example, Freund's incomplete Adjuvant and Complete Adjuvant (Difco Laboratories, Detroit, Mich.); Merck Adjuvant 65 (Merck and Company, Rahway, N.J.); AS-2 (GlaxoSmithKline, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

The adjuvant can induce an anti-inflammatory immune response (antibody or cell-mediated). Accordingly, high levels of anti-inflammatory cytokines (anti-inflammatory cytokines may include, but are not limited to, interleukin 4 (IL-4), interleukin 5 (IL-5), interleukin 10 (IL-10), and transforming growth factor beta (TGFβ). Optionally, an anti-inflammatory response can be mediated by CD4+ T helper cells. Bacterial flagellin has been shown to have adjuvant activity (McSorley el al., J. Immunol. 169:3914-19, 2002). Also disclosed are polypeptide sequences that encode flagellin proteins that can be used in adjuvant compositions. Additional adjuvants include but are not limited to, monophosphoryl lipid A (MPL), aminoalkyl glucosaminide 4-phosphates (AGPs), including, but not limited to RC-512, RC-522, RC-527, RC-529, RC-544, and RC-560 (Corixa, Hamilton, Mont.).

Additional illustrative adjuvants for use in the disclosed compositions (e.g., vaccines) include, for example, a combination of monophosphoryl lipid A, preferably 3-de-O-acylated monophosphoryl lipid A, together with an aluminum salt adjuvants available from Corixa Corporation (Seattle, Wash.; see, for example, U.S. Pat. Nos. 4,436,727; 4,877,611; 4.866,034, 4,912,094); CpG-containing oligonucleotides (in which the CpG dinucleotide is unmethylated; see e.g., WO 96/02555, WO 99/33488 and U.S. Pat. Nos. 6,008,200, 5,856,462); immunostimulatory DNA sequences (see e.g., Sato et al., Science 273:352, 1996); saponins such as Quil A, or derivatives thereof, including QS21 and QS7 (Aquila Biopharmaceuticals Inc., Framingham, Mass.); Escin; Digitonin; or Gypsophila or Chenopodium quinoa saponins; Montamide ISA 720 (Seppic, France), SAF (Chiron, Calif., United States), ISCOMS (CS), MF-59 (Chiron), the SBAS series of adjuvants (e.g., SBAS-2 or SBAS-4, available from GlaxoSmithKline, Philadelphia, Pa.), Detox (Enhanzynr™) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826, 09/074,720: polyoxyethylene ether adjuvants such as those described in WO 99/52549A 1; and combinations thereof. In some embodiments, the adjuvant comprises alpha Galactosylceramide.

In some aspects, the nanoparticle comprises structurally modified (arginine/histidine/disulfide) amphiphilic chitosan-lipid polymers (CL-NPs) with RIG-d adjuvant/cytosine phosphoguanine (PUUC/CpG) loaded on the particle surface and R848 was encapsulated inside the core.

It is understood and herein contemplated that the disclosed nanoparticles can be used in the construction of a vaccine against microbial infection. In one aspect, disclosed herein are vaccines comprising the nanoparticle of any preceding aspect and one or more immunogenic nucleic acids, polynucleotide, peptides, antibody, protein, inactivated virus, killed virus, viral particle, or any combination thereof.

The immunogen used in the vaccine to generate an immune response can be a single immunogen or multiple immunogens (i.e., multivalent). Accordingly, the vaccine can comprise a single (i.e., one), 2, 3, 4, 5, 6, 7, 8, 9, or 10 immunogenic nucleic acids, polynucleotides, peptides, proteins, antibodies, viral particles, inactivated viruses, and/or killed viruses, or any combination thereof When multiple immunogenic nucleic acids, polynucleotides, peptides, proteins, antibodies, viral particles, inactivated viruses, and/or killed viruses, or any combination thereof are present, the nucleic acids, polynucleotides, peptides, proteins, antibodies, viral particles, inactivated viruses, and/or killed viruses, or any combination thereof can be specific for the same or different epitopes (i.e., a multivalent vaccine). Thus, in one aspect, disclosed herein are vaccines, wherein the more than one or more (i.e., 2, 3, 4, 5, 6, 7,8 9, 10) immunogenic nucleic acids (including, but not limited to a siRNA molecule, a miRNA molecule, a shRNA molecule, a pDNA molecule), polynucleotides, peptides, proteins, antibodies, viral particles, inactivated viruses, and/or killed viruses are immunogenic against a first epitope. That is, the immunogenic nucleic acids, polynucleotides, peptides, proteins, antibodies, viral particles, inactivated viruses, and/or killed viruses illicit immune responses against the same epitope. However, as noted above, the immunogenic nucleic acids (including, but not limited to a siRNA molecule, a miRNA molecule, a shRNA molecule, a pDNA molecule), polynucleotides, peptides, proteins, antibodies, viral particles, inactivated viruses, and/or killed viruses can also illicit immune responses against multiple different epitopes. Accordingly, disclosed herein are vaccines, wherein the more than one immunogenic nucleic acids, polynucleotides, peptides, proteins, antibodies, viral particles, inactivated viruses, and/or killed viruses are immunogenic against a first epitope and at least one second epitope. In one aspect, the first and second epitopes are the same. In another aspect the first and second epitopes are different.

The disclosed nanoparticles and vaccines can also be used as part of a treatment regimen to facilitate the treatment, reduction, inhibition, decrease, amelioration, and/or prevention of a microbial infection. Thus, disclosed herein are antimicrobial treatment regimens comprising administering one or more (i.e., 2, 3, 4, 5, 6, 7,8 9, 10) vaccines disclosed herein and/or one or more (i.e., 2, 3, 4, 5, 6, 7,8 9, 10) of the nanoparticles disclosed herein and a separate vaccine.

In one aspect, disclosed herein are antimicrobial treatment regimens, wherein the vaccine comprises one or more (i.e., 2, 3, 4, 5, 6, 7,8 9, 10) immunogenic nucleic acids (including, but not limited to a siRNA molecule, a miRNA molecule, a shRNA molecule, a pDNA molecule), polynucleotides, peptides, proteins, antibodies, inactivated viruses, killed viruses, viral particles, or any combination thereof.

Also disclosed herein are antimicrobial treatment regimens, wherein the treatment regimen comprises the administration at least two vaccines, a first vaccine and a second vaccine. In one aspect, each vaccine comprises a single immunogenic nucleic acid, polynucleotide, peptide, protein, antibody, viral particle, inactivated virus, or killed virus. In one aspect, the vaccine is multivalent.

In one aspect, the first and second vaccines are administered at the same or different times. For example, the first and second vaccines can be a prime/boost regimen with the second vaccine administered at least 7, 8, 9, 10, 11, 12, 13, 14, 15,1 6,17,18, 19, 20, 21, 22, 23, 24, 25, 26, 27, 28, 29, 30, 31, 35, 40, 45, 50. 55, 60, 65, 70, 75, 80, 85, 90 days, 3, 4, 5, 6, 7, 8,9, 10,11,12,13,14,15,16,17,18, 19,20,21,22,23,24,25,26,27,28,29,30,31,32,33, 34, 35, or 36 months after administration of the first vaccine. In some instances, the first and second vaccines are the same or different immunogenic agents (i.e., nucleic acid (including, but not limited to a siRNA molecule, a miRNA molecule, a shRNA molecule, a pDNA molecule), polynucleotide, peptide, protein, antibody, inactivated virus, killed virus, or viral particles) but eliciting an immune response against the same epitope. In some instances, the first and second vaccines are the same or different immunogenic agents (i.e., nucleic acid (including, but not limited to a siRNA molecule, a miRNA molecule, a shRNA molecule, a pDNA molecule), polynucleotide, peptide, protein, antibody, inactivated virus, killed virus, or viral particles) but eliciting an immune response against the different epitopes of the same microbial infection.

### A. Method of Treating Airborne or Pulmonary Infections

The nanoparticles disclosed herein can be used to generate immune responses against microbial pathogens that infect the lungs. Pulmonary infections include coronavirus, influenza, pneumonia, and other viruses, bacteria, fungi, and parasites that infect the lungs. Pulmonary infections can further include empyema, lung abscess, tuberculosis, chronic obstructive pulmonary disease (COPD), cystic fibrosis, bronchitis, bronchiolitis, or asthma. For example, the disclosed nanoparticles can be used to treat, inhibit, reduce, decrease, ameliorate, and/or prevent a infection with a Rhinovirus, Coronavirus (including, but not limited to avian coronavirus (IBV), porcine coronavirus HKU15 (PorCoV HKU15), Porcine epidemic diarrhea virus (PEDV), ICoV-229E, HCoV-OC43, HCoV-HKU1, HCoV-NL63, SARS-CoV, SARS-CoV-2 (including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1A/AY.2 (Delta plus), SARS-CoV-2 with D614G, P681H, and D950N mutations in B.1.621 (Mu), SARS-CoV-2 with G75V, T76I, Δ246-252, L452Q, F490S, D614G, and T859N mutations in C.37 (Lambda), SARS-CoV-2 with T478K, Q498R, and H655Y mutations in B.1.1.529 (Omicron)), or MERS-CoV), Influenza virus A, Influenza virus B, Measles virus, Varicella-Zoster virus, Respiratory syncytial virus, Mumps virus, Adenovirus, Rotavirus, Ebola virus, Marburg virus, Lassa fever virus, M1,cobacterium tuberculosis, Mycobacterium hovis, Mycobacteriumn bvis strain BCG. BCG substrains, Mycobacterium avium, Mcobacterium intracellular, Mvcobacterium africanum. Mycobacterium kansasii, Mycobacterium marinum, Mycobacterium ulcerans, Mycobacterum avium subspecies parauberculosis, Mycobacterium chimaera, Bacillus anthracis. Bordetella avium, Bordetella pertussis, Bordetella bronchiseptica, Bordetella trematum, Bordetella hinzii, Bordetella pteri, Bordetella parapertussis, Bordetella ansorpii other Bordetella species, Corynebacterium diphtheriae, Aspergillusfumigatus. Accordingly, disclosed herein are methods of treating, inhibiting, reducing, decreasing, ameliorating, and/or preventing a pulmonary infection comprising administering a therapeutically effective amount of any of the nanoparticles disclosed herein to a patient in need thereof. For example, disclosed herein are methods of treating, inhibiting, reducing, decreasing, ameliorating, and/or preventing a pulmonary infection (such as, for example an infection with a Rhinovirus, Coronavirus (including, but not limited to avian coronavirus (IBV), porcine coronavirus HKU15 (PorCoV HKU15), Porcine epidemic diarrhea virus (PEDV), HCoV-229E, HJCoV-OC43, HCoV-HKU1, HCoV-NL63, SARS-CoV, SARS-CoV-2 (including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY2 (Delta plus), SARS-CoV-2 with D614G, P681H, and D950N mutations in B.1.621 (Mu), SARS-CoV-2 with G75V, T76I, A246-252, L452Q, F490S, D614G, and T859N mutations in C.37 (Lambda), SARS-CoV-2 with T478K, Q498R, and 1H655Y mutations in B.1.1.529 (Omicron)), or MERS-CoV), Influenza virus A, Influenza virus B, Measles virus, Varicella-Zoster virus, Respiratory syncytial virus, Mumps virus, Adenovirus, Rotavirus, Ebola virus, Marburg virus, Lassa fever virus, Mycobacterium tuberculosis, Mycobacterium bovis. Mycobacterium bovis strain 8CGN, BCG substrains, Mycobacterium avium, Mycobacterium intracellular, Mycobacterium africanum, Mvcobacterium kansasii, 4M,cobacterium marinum, Mycobacterium ulcerans. Mvcobacterium avium subspecies paratubercuslosis, Acobacteriun chimaera, Bacillus anthracis, Bordetella avium, Bordetella pertussis, Bordetella bronchiseptica, Bordetella trematum, Bordetella hinzii, Bordetella pteri, Bordetella parapernussis, Bordetella ansorpii other Bordetella species, Corynebacterium diphtheriae, AsImigillus fumigatus) comprising administering to a patient in need thereof a therapeutically effective of a nanoparticles comprising a polymer having an outer surface and an inner core, wherein the polymer comprises a polysaccharide (such as, for example chitosan), a lipid, and an amino acid (including but not limited to arginine at 0% to 100% by weight of total amino acids and/or histidine at 0% to 100% by weight of total amino acids), wherein the lipid and amino acid are conjugated to the polysaccharide. The nanoparticle can comprise an outer surface of the nanoparticle is loaded with a first agent (such as, for example, a nucleic acid, a polynucleotide, peptide, protein, a siRNA molecule, a miRNA molecule, a shRNA molecule, a pDNA molecule, or any combination thereof) and/or and inner core of the nanoparticle is loaded with a second agent (such as, for example, a small molecule, immune adjuvants, fluorochrome, contrast agents including, but not limited to hydrophobic agents).

When treating, inhibiting, reducing, decreasing, ameliorating, and/or preventing a pulmonary infection, the nanoparticle can comprise an antigen to generate an immune response against the infecting pathogen or an effector molecule like siRNA, shRNA, miRNA or small molecule. In some aspects, the immunizing agent (i.e., peptide, protein, siRNA, miRNA, polynucleotide, shRNA, vaccine, or antibody) is not comprised in the nanoparticle, but administered in a composition with the nanoparticle or in a separate composition administered concurrently, before, or after the administration of the nanoparticle. Whether comprising the immunizing agent or not, the nanoparticle can act as an adjuvant enhancing the immune response to the antigen, shRNA, siRNA, miRNA, polynucleotide, peptide, protein, antibody, or vaccine.

In some aspects, the nanoparticle is administered via an intramuscular route, an intranasal route, or any combination thereof.

In one aspect, the methods disclosed herein comprise a first and second vaccines are administered at the same or different times. For example, the first and second vaccines can be a prime/boost regimen with the second vaccine administered at least 7, 8, 9, 10, 11, 12, 13, 14, 15,1 6,17,18, 19, 20, 21, 22, 23, 24, 25, 26, 27, 28, 29, 30, 31, 35, 40, 45, 50, 55, 60, 65, 70, 75, 80, 85, 90 days, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after administration of the first vaccine. In some instances, the first and second vaccines are the same or different immunogenic agents (i.e., nucleic acid (including, but not limited to a siRNA molecule, a miRNA molecule, a shRNA molecule, a pDNA molecule), polynucleotide, peptide, protein, antibody, inactivated virus, killed virus, or viral particles) but eliciting an immune response against the same epitope. In some instances, the first and second vaccines are the same or different immunogenic agents (i.e., nucleic acid (including, but not limited to a siRNA molecule, a miRNA molecule, a shRNA molecule, a pDNA molecule), polynucleotide, peptide, protein, antibody, inactivated virus, killed virus, or viral particles) but eliciting an immune response against the different epitopes of the same microbial infection.

As noted herein, the disclosed nanoparticles can be used to treat a Coronaviral infection. Coronavirus can include, but is not limited to, avian coronavirus (IBV), porcine coronavirus HKU15 (PorCoV IHKU15), Porcine epidemic diarrhea virus (PEDV), HCoV-229E, HCoV-OC43, HCoV-HKUI, HCoV-NL63, SARS-CoV, SARS-CoV-2 (including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS25 CoV-2 with T487K, P681 R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, A246-252, L452Q, F490S, D614G, and T859N mutations in C.37 (Lambda), SARS-CoV-2 with T478K, Q498R, and H655Y mutations in B.1.1.529 (Omicron)), or MERS-CoV).

Severe acute respiratory syndrome coronavirus 2 (SARS-CoV-2) is a type of huma coronavirus. Representative examples of human coronavirus can also include, but are not limited to, human coronavirus 229E (HCoV-229E), human coronavirus 0C43 (HCoV-OC43), human coronavirus HKUI (HCoV-HKU1), Human coronavirus NL63 (HCoV-NL63), severe acute respiratory syndrome coronavirus (SARS-CoV), and Middle East respiratory syndrome-related coronavirus (MERS-CoV). Accordingly, disclosed herein are methods of treating, reducing, decreasing, inhibiting, ameliorating, and/or preventing a human coronavirus infection (such as, for example, SARS-CoV-2 (including, but not limited to the SARS-CoV-2 B1.351 variant, SARS-CoV-2B.1.1.7 (alpha), SARS-CoV-28.1.1.7 variant mutant N501Y (alpha), SARS-CoV-2 delta variant, SARS-CoV-2 P.1 variant, SARS25 CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T761, A246-252, L452Q, F490S, D614G, and T859N mutations in C.37 (Lambda), SARS-CoV-2 with T478K, Q498R, and H655Y mutations in B.1.1.529 (Omicron)) comprising administering to a subject a lipid-functionalized chitosan-based stabilized Spike-protein nanovaccine, co-loaded with TLR (R848, CpG) and RNA-based RIG-I-like receptor as adjuvants. In some aspects, nanovaccine comprise structurally modified (arginine/histidine/disulfide) amphiphilic chitosan-lipid polymers (CL-NPs) with RIG-1 adjuvant/cytosine phosphoguanine (PUUC/CpG) loaded on the particle surface and R848 was encapsulated inside the core. The vaccine can further comprise, along with the adjuvant-loaded PLP, a stabilized spike protein

In some embodiments, the coronavirus infection can be caused by an avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV.

As used herein, “COVID-19” refers to the infectious disease caused by SARS-CoV-2 and characterized by, for example, fever, cough, respiratory symptoms, rhinorrhea, sore throat, malaise, headache, chills, repeated shaking with chills, diarrhea, new loss of smell or taste, muscle pain, or a combination thereof

In some embodiments, the subject with a coronavirus exhibits one or more symptoms associated with mild COVID-19, moderate COVID-19, mild-to-moderate COVID-19, severe COVID-19 (e.g., critical COVID-19), or exhibits no symptoms associated with COVID-19 (asymptomatic). It should be understood that in reference to the treatment of patients with different COVID-19 disease severity, “asymptomatic” infection refers to patients diagnosed with COVID-19 by a standardized RT-PCR assay that do not present with fever, cough, respiratory symptoms, rhinorrhea, sore throat, malaise, headache, or muscle pain.

In some embodiments, the subject with a coronavirus exhibits one or more symptoms selected from dry cough, shortness of breath, and fever. In other embodiments, the subject exhibits no symptoms associated with COVID-19 but has been exposed to another subject known or suspected of having COVID-19.

### B. Methods of Making Nanoparticles

Also disclosed herein in one aspect are methods of making any of the nanoparticles disclosed herein comprising a) carboxylating the polysaccharide; b) thiolating the polysaccharide; c) forming disulfide with a cysteamine; d) conjugating the amino acid using carbodiimide chemistry; e) conjugating stearyl amine using carbodiimide chemistry; f) deprotecting a tert-Butyloxycarbonyl group with trifluoroacetic acid; g) sonicating the nanoparticle; and h) purifying the nanoparticle with dialysis. In some aspect, the method further comprises loading the nanoparticle with the second therapeutic agent.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### 1. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, scFv, and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992). 128. As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Cur. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### 2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447451, (1991); Bagshawe, K. D., Br. J. Cancer, 60:275-281, (1989); Bagshawe, et al., Br. J. Cancer, 58:700-703, (1988); Senter, et al., Biocontjugate Chem., 4:3-9, (1993); Battelli, et al., Cancer Immunol. Immuno/her., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, D)NA and Cell Biology 10:6,399409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A.R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops. suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable..

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about I pg/kg to up to 100 mg/kg of body weight or more per day, depending on the factors mentioned above.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: a Lipid-Polysaccharide Based Multi-Adjuvant Intramuscular-Prime Intranasal Boost SARS-CoV-2 Subunit Nanovaccine Generates Strong Systemic and Mucosal Immune Response in Mice

### a) Introduction

The COVID-19 has made the development of vaccines and therapies an immediate priority worldwide. Even though the current mRNA vaccines have shown excellent efficacy and safety, several limitations have become apparent. These include the breadth and durability of protective immunity, efficacy against emerging variants, inadequate protection from infection and transmission, and lack of mucosal immunity in the lung and nasopharynx. Combination adjuvants are known to induce broadened and synergistic immune responses and can help in long-term protection against SARS-CoV-2 variants. Combination adjuvants on lipid-modified polysaccharide-based subunit nanovaccines can modulate innate and adaptive immune responses against SARS-CoV-2. A lipid-functionalized chitosan-based stabilized Spike-protein nanovaccine, co-loaded with TLR (R848, CpG) and RNA-based RIG-1-like receptor as adjuvants was developed. A heterologous vaccination strategy with intramuscular priming (IM) followed by intranasal (IN) boosting was examined.

### b) Methods

Degradable nanovaccines were synthesized using structurally modified (arginine/histidine/disulfide) amphiphilic chitosan-lipid polymers (CL-NPs). RIG-I adjuvant/cytosine phosphoguanine (PUUC/CpG) was electrostatically loaded on the particle surface, and R848 was encapsulated inside the core. For in vivo studies, mice were injected with adjuvant-loaded PLPs and stabilized spike protein in a heterologous manner (e.g., EM prime and IN boost, 3 weeks after priming). Mice were sacrificed on day 36, and lungs, blood, and BAL fluid were collected. Antibody titers were measured in serum and BAL fluid via ELISA. Immune cell phenotypes were analyzed by flow cytometry.

### c) Results

When delivered in vivo, CL-CpG+PUUC NPs generated strong anti-spike IgA levels in the BAL fluid (FIG. 1a) and induced strong humoral immune responses, which were characterized by anti-spike IgG and neutralizing antibodies (FIGS. 1b and 1c). CL-CpG+PUUC NPs also increased the cell populations with tissue-resident memory T cell markers (CD3+CD69⁺CD103*) in the lung tissue (FIG. 1d). Combination adjuvant-NPs targeting TLR, RIG-I, and cytokine secretion (IL-12p70, IL-1β, IFN-β) in APCs cultured in vitro. Specifically, TLR-RLR-STING NPs showed differential and robust mucosal immune responses in vivo.

### d) Conclusion

The results herein suggest that SARS-CoV-2-mimicking adjuvanted subunit vaccines with heterologous immunization techniques can lead to robust and lung-specific protective immunity against SARS-CoV-2 and may help in reducing transmission and enhance protection for future emerging variants.

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods and compositions of the appended claims are not limited in scope by the specific methods and compositions described herein, which are intended as illustrations of a few aspects of the claims and any methods and compositions that are functionally equivalent are intended to fall within the scope of the claims. Various modifications of the methods and compositions in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

### 2. Example 2: a Multiadjuvants Polysaccharide-Amino Acid-Lipid (PAL) Subunit Nanovaccine Generates Robust Systemic and Lung-Specific Mucosal Immune Responses Against SARS-CoV-2 in Mice

The COVID-19 pandemic continues to cause a global health crisis with frequent viral mutations and uncontrolled transmission in many parts of the world. In the U.S. alone, weekly death rates from COVID-19 remain well over 1000, even three years after the start of the pandemic. Almost 72% of the world's population have received the licensed parenteral mRNA-LNP and adenoviral vectors-based vaccines. These approved vaccines are designed to be administered via intramuscular (IM) route, show high efficacy against severe SARS-CoV-2 infections, and reduce hospitalization and deaths. Unfortunately, recent studies have shown that even after vaccination with booster doses, there are asymptomatic, symptomatic, and some severe cases of SARS-CoV-2 infection observed after a few months. These results indicate declining antiviral immunity within a short period and raise questions about durable efficacy and protection through these vaccines. Moreover, continuous viral mutation (VOC: variants of concern) that evades the immune response also results in reduced vaccine effectiveness. Additionally, vaccine accessibility and acceptance remain a significant concern.

The SARS-CoV-2 pathogen infects human cells through binding of its RBD (Receptor Binding Domain) region to the AC&2 receptors present in the cells of respiratory tract (mucosal tissues), which makes SARS-CoV-2 primarily a mucosal pathogen. However, IM vaccinations predominantly induce systemic immune responses (circulating antibodies, memory B cells, effector T cells), with limited mucosal immunity at the sites of infection, i.e., nasopharynx and lungs. The immune response generated after IM immunization leaves the upper respiratory tract vulnerable to viral replication and dissemination, leading to reduced sterilizing immunity through IM vaccines. Collectively, approved vaccines have limited efficacy in preventing SARS-CoV-2 infection due to several factors, including poor respiratory mucosal immunity, viral immune evasion, and increased viral transmission. Mucosal vaccination can be one of the solutions to the above problems. It can generate both mucosal and systemic antiviral immune responses (humoral and cellular) similar to a natural infection and can ultimately lead to better protection and reduced transmission.

The antiviral mucosal immunity is characterized by the generation of robust mucosal IgA, IgG, and neutralizing antibodies in the bronchoalveolar lavage (BAL) fluid, lung-specific T cell immunity (Tissue-resident memory T and B cell) with TH1 type and systemic immune responses. Vaccine administration through the respiratory route (nasal/oral vaccines) generates more potent tissue-level mucosal immunity against SARS-CoV-2, compared to the IM route, thus emerged as an effective approach for new mucosal vaccines. One can either boost the already IM vaccinated population with the intranasal vaccine (heterologous vaccine strategy), which can also strengthen the circulating immunity achieved via intramuscular priming (IM-Prime/IN-Boost), or use them for both priming and boosting (homologous strategy, IN-Prime/IN-Boost) in the unvaccinated population, which can increase compliance and acceptance. Recently, encouraging preclinical findings have emerged utilizing the IM-Prime/IN-Boost approach. One study involved systemic priming with an mRNA-LNP vaccine followed by an intranasal boost of spike protein, while another study administered a Diphtheria toxin-conjugated vaccine through IM-Prime and IN-Boost. This vaccination strategy can enhance systemic immunity through mucosal boost (a prime-pull mechanism) and help achieve sterilizing immunity against SARS-CoV-2.

As of December 2022, among a total of 21 mucosal vaccines in trials, five have been authorized for use or registered for regulatory agency review for SARS-CoV-2. Out of which, three are viral vector-based vaccines: Bharat Biotech in India, Gamaeleya in Russia, and CanSino Biologics in China. However, the effectiveness of these viral vector-based vaccines for the worldwide population is still under assessment. Indeed, the fact that none of these vaccines have been authorized for use in the United States or Europe, nor has the WHO granted them emergency use listing, could be attributed to the limited data available from clinical efficacy trials to assess their impact on transmission, infection, or disease. In rare cases, for live-attenuated vaccines, there might be a potential risk of returning to their virulent state. Mucosal vaccines for influenza that utilized intranasal live attenuated pathogens had been approved but were not 100% effective and safe. Therefore, there is a need to develop new mucosal vaccine strategies that not only overcome the limitations of IM vaccines and achieve high levels of durable protection against infection but also mitigate the post-COVID severe effects, such as post-acute sequelae of SARS-CoV-2 infection and long COVID.

Protein subunit vaccines are known to be comparatively safer as they use only a fragment of a pathogen. Some promising results with recombinant protein subunit vaccines have been recently reported. However, protein subunit vaccines exhibit less immunogenicity requiring the use of vaccine adjuvants, which are often delivered using biomaterial-based polymeric nanoparticle (NP) formulations, often known as nanovaccines. Adjuvants on nanoparticles increase antigen immunogenicity by activating pattern recognition receptors (PRRs) of innate immune system, modulating the antigen pharmacokinetics, and facilitating antigen dose sparing. Several studies have shown that combination of adjuvants in a vaccine formulation can elicit synergistic, complementary, and antagonistic effects on innate and adaptive immunity. Several adjuvants that target multiple PRRs, including RLRs (retinoic inducible gene 1: (RIG-I)-like receptors) and Toll-like receptors (TLRs: 4, 7/8, 9), either alone or in combination, have been utilized in studies related to SARS-CoV-2 mucosal vaccines. While these adjuvants have shown promising results in animal models, they are not able to provide complete protection against infection. However, RIG-1 agonists are mainly used to enhance antiviral immunity in other viral infections such as influenza or the west nile virus.

Here we designed a novel, multiadjuvanated protein subunit SARS-CoV-2 nanovaccine formulation using different combinations of PRR-agonists-PUUC RNA: RLRs agonist, CpG DNA: TLR9 agonist and R848: TLR7/8 agonist, which are loaded on degradable, polysaccharide- amino acid-lipid polymeric nanoparticles (PAL-NPs). We first evaluated the best combination adjuvant formulation that can enhance both the mucosal and systemic immunity against SARS-CoV-2 via the intramuscular (IM) prime and intranasal (IN) boost (IM-Prime/IN-Boost) strategy. We found that PAL-NPs with PUUC (RIG-I agonist) and CpG DNA (TLR9 agonist), combined with the recombinant stabilized Spike S1 trimer protein, induced robust mucosal and systemic humoral and local cellular immunity (TH i response) with IM-Prime/IN-Boost group. However, the IN-Prime/IN-Boost group also induces robust lung T cell-mediated immunity, higher than the IM-Prime/!N-Boost group, and a comparable mucosal humoral response (IgA and neutralizing antibodies), which indicates that a mucosal delivery route can be attainable for future vaccines compared to only parenteral route.

### A) RESULTS

### (1) Synthesis and Characterization of Multiadjuvanated PAL-NP Vaccine Formulations

We developed a Mult adjuvanted nanoparticle-based SARS-CoV-2 protein subunit vaccine using a newly designed polymer-lipid molecule (FIG. 12). The use of polymer particles for delivery of combination adjuvants and antigens, both in vitro and in vivo. Proper selection of polymers is necessary for better loading and delivery of multiple charged or hydrophobic adjuvants on the polymer nanoparticles. Synthetic amphiphilic polymers can fulfill these criteria because they are chemically modified with the desired functional group moieties, specific for adjuvant loading and delivery. Therefore, we first synthesized the amphiphilic polymer-lipid [OCMC-S-S-(A/H)-SA], namely- polysaccharide-amino acid-lipid (PAL polymer), by sequential, structural, and chemical modification of polysaccharide (chitosan Mw: 15 KDa) (FIG. 12A, fig. SIA, and supplementary text 2.1). The PAL polymer synthetic steps start with (i) carboxylation at C-6 position using mono-chloroacetic acid in slight basic medium, (ii) thiolation of C-2 amine groups with thioglycolic acid using carbodiimide chemistry, (iii) disulfide formation between thiols at C-2 position of carboxylated chitosan and the thiols of cysteamine, (iv) carbodiimide conjugation of the carboxyl group of N-α Boc protected amino acids (arginine and histidine) with amine group of cysteamine (v) stearyl amine conjugation at C-6 carboxyl group (0-substitution) using carbodiimide chemistry, (vi) final deprotection of Boc groups. Purification of all steps was performed by dialysis or precipitation and followed by lyophilization at the required pH. The incorporation of functional groups in polysaccharides is confirmed by proton NMR spectroscopy. We confirm the conjugation and incorporation of amino acids and stearyl chains (lipid) into the chitosan backbone by comparing the ¹HNMR spectra of thiolated chitosan polysaccharide and the final modified polysaccharide-amino acid-lipid polymer (FIG. 12B). The appearance of a characteristic peak at 7.1-7.4 ppm is due to a guanidine functional group in arginine, indicating the successful arginine grafting in polysaccharide. Additionally, a characteristic peak of imidazole ring protons at 8.6 ppm and 7.6 ppm confirms histidine grafting on the polysaccharide backbone. The peaks at 1.2 ppm (—CH2—) and 1.6 ppm (—CH3) in the ¹H NMR spectrum of amphiphilic chitosan polymer confirm the successful incorporation of the stearyl chain in chitosan. The presence of disulfide bond formation was confirmed by Elmann's assay, which shows a total reduction in free thiol concentration after cysteamine conjugation (rig. SIB).

We further synthesized a cationic and degradable nanoparticle from amphiphilic PAL polymer, known as PAL-NP, using the probe sonication method in DMSO/water mixture and purification by dialysis. Multiadjuvanated PAL-NPs were prepared by the loading of different adjuvant combinations of PUUC RNA (targeting RIG-1-like receptors: retinoic acid-inducible gene 1), CpG DNA (targeting TLR9) and R848 (targeting TLR7/8), either by surface electrostatic adsorption (anionic adjuvants: PUUC and CpG) or by encapsulation (hydrophobic adjuvants: R848) inside the nanoparticles' lipid core (FIG. 12C and Table 1). The blank and R848 loaded PAL-NPs have an average hydrodynamic size of −250 nm and zeta potential of ˜+30 mV at pH ˜7.0, which makes it appropriate for surface loading of nucleic acid adjuvants (FIG. 12D). Transmission electron microscopy (TEM) was used to determine the morphology of PAL-NPs, revealing a well-dispersed spherical shape with the average diameter between 150-200 nm (FIG. 12D, inset). The degradability of PAL-NPs mediated by disulfide bond reduction was confirmed by a decrease in nanoparticle size from −200 nm to −50 nm with the addition of dithiothreitol (DTT) for 24 h (FIG. S1C), which was not observed in disulfide-bond deficient PAL-NPs (non- degradable PAL-NPs).

To examine the immunostimulatory effects of different combinations of PRR agonists (RIG-I agonist and TLRs agonist) in vitro, we screened eight PAL-NPs adjuvant formulations (CpG, PUUC, CpG+PUUC, R848, R848+CpG, R848+PUUC, R848+CpG+PUUC) (FIG. 12, E to G). These formulations were incubated with murine bone-marrow-derived dendritic cells (BMDCs) generated using GM-CSF cytokines for 7 days in a 96-well plate (Table 1). After 24 h, the collected supernatants were analyzed to quantify proinflammatory cytokine secretion. In vitro studies show that PUUC+CpG PAL-NPs significantly increased the secretion of proinflammatory cytokine IL-1β (FIG. 12E). However, IL-1β secretion is mainly driven by CpG PAL-NPs. Similarly, the IFN- secretion is also CpG driven, but interestingly. R848+CpG PAL- NPs synergistically increase the IFN-0 secretion (FIG. 12F). R848 PAL-NPs is the only group that significantly stimulates the secretion of proinflammatory cytokine IL12p70 (FIG. 12G). Furthermore, none of the triple adjuvanated PAL-NPs enhanced proinflammatory cytokine secretion. Surprisingly, only blank PAL-NP, which are considered as a control group due to the absence of real RLR and TLR agonist adjuvants, also show considerable IL-1P secretion but do not stimulate the secretion of the IFN-β and IL12p70. However, both PUUC+CpG PAL-NPs and R848+CpG PAL-NPs enhance the stimulation of the initial innate immune response.

(2) PAL-NPs Protein Subunit Vaccine Adjuvanated with RIG-1 (PUUC) and TLR9 (CpG) Agonists Elicit Robust SARS-CoV-2 Mucosal and Systemic Humoral Immune Responses, when Delivered IM-Prime/IN-Boost

For boosting the current SARS-CoV-2 vaccine or developing a mucosal vaccine, an ideal vaccine candidate should have an appropriate adjuvant combination that generates potent and balanced mucosal and systemic immunity. Therefore, we first performed the in vivo screening of multiple adjuvant combinations on PAL-NPs and evaluated the best adjuvant combination that enhances the mucosal and systemic SARS-CoV-2 immune response. We selected four adjuvanated (PUUC, R848, R848+CpG, PUUC+CpG) PAL-NPs groups and administered them in mice through IM-Prime IM-Boost strategy (FIG. 13A). These nanovaccine formulations were prepared by loading/encapsulation the adjuvants (PUUC, R848, R848+CpG, PUUC+CpG) on PAL-NPs and mixing them with stabilized recombinant SARS-CoV-2 S1 trimer subunit as the target antigen. S1 trimer subunit is more immunogenic than the RBD alone due to the presence of other epitopes at the outer part of the RBD, which contribute to the neutralization. The blank NPs (no real adjuvants) in our study exhibit minimal immune responses; therefore, we have considered them as the control group along with PBS. Mice were immunized via the IM-Prime at day 0 and boosted via IN route at day 21 with PAL-NPs vaccine formulations and sacrificed at day 35 (14 days post- boost), and BAL fluid and serum samples were collected for analyzing the generated mucosal and systemic humoral response. We first quantified the mucosal anti-spike S1 IgA and IgG levels in the BAL fluid. We found that PUUC+CpG PAL-NP group significantly increases BAL anti-spike IgA and IgG levels at 1:10 dilution compared to other formulations, including control groups (FIGS. 13B and 13C). The secreted mucosal IgA and IgG levels in BAL fluid of mice vaccinated with adjuvanated PAL-NPs vaccine formulations and controls follow the order: PUUC+CpG>PUUC>R848>R848+CpG>PAL-NPs>PBS. To assess TH1/TH2 skewed IgG response in BAL fluid elicited through adjuvanated PAL-NPs vaccine immunization, we evaluated IgG subsets (IgG1 and IgG2a) present (FIGS. 13D and 13E). The TH1-associated IgG2a levels were significantly higher in mice immunized with PUUC+CpG PAL-NP group than in the other groups (FIG. 13E). The IgG2a/IgG1 ratio indicates that both PUUC+CpG and R848+CpG groups show the TH 1-biased response, but a comparatively higher response was observed in the former (FIG. 13F). This data indicates that PUUC+CpG PAL-NPs vaccine formulation generates more potent mucosal IgA and IgG in BAL fluid.

Further, we assessed serum systemic IgG and neutralizing antibody (nAb) responses (FIG. 13G, 13H, and 13I). The PUUC+CpG PAL-NPs group resulted in significantly increased levels of anti- SARS-CoV-2 IgG in serum at 10-fold dilution (FIG. 132G). The serum IgG levels generated (Area under the curve: AUC) after administration of adjuvanated PAL-NPs formulation and control groups follow this order: PUUC+CpG>PUUC>R848+CpG>R848=PAL-NPs (FIG. 13H). nAbs play a crucial role in reducing the replication of SARS-CoV-2 and are essential in protecting against severe infections caused by the virus. We assessed generated serum nAbs by measuring the inhibition of the Spike RBD-ACE2 interaction using an ELISA-based ACE2 competition assay. We found that PUUC+CpG PAIL-NPs group elicited high titers of neutralizing antibodies. Spike protein neutralization was detectable at up to a 10r-fold dilution in PUUC+CpG PAL-NPs group, which is higher than any other groups (lower absorbance value: A450-A630), and the total neutralization was observed at 100-fold sera dilution (FIG. 13H). To evaluate TH1/TH2 skewed IgG response, elicited through IM-Prime/IN-Boost immunization, we assessed the serum IgG subsets (IgG1 and IgG2a) present. We observe that among various PRR agonist formulations on PAI-NP, the PUUC+CpG and PUUC group alone, demonstrated the similar and highest induction of total IgG1 [FIGS. 13J and 13K (IgG1 dilution curve and AUC)]. We also observe that only PUUC+CpG PAL-NP group significantly induces highest anti-spike IgG2a titers [FIGS. 13L and 13M (IgG2a dilution curve and AUC)]. However, the ratio of IgG2a/IgG1 indicates that PUUC+CpG on PAL-NPs show the highest TH1 biased antibody response compared to other adjuvant groups (FIG. 13N). Thus, PUUC+CpG PAL-NPs vaccine formulation shows more potent mucosal IgA and IgG levels in BAL fluid and serum. These findings indicate that the multiadjuvants PUUC+CpG PAL-NPs elicit a stronger mucosal antibody (IgA and IgG) response, systemic antibody response, and neutralizing antibody titers, highlighting their significant use in mucosal subunit nanovaccines.

(3) PAL-NPs Protein Subunit Vaccine Adjuvanated with RIG-I (PUUC) and TLR9 (CpG) Agonists Induces Robust SARS-CoV-2 Mucosal T Cell and B Cell Immune Responses, when Delivered IM-Prime/IN-Boost

The adaptive cellular immune responses were generated and functional in the local tissues during respiratory infection and are responsible for providing long-lasting protective immunity at the infection sites. Accordingly, we investigated the pulmonary T cell and B cell responses on 35^{th} day (14 days post-boost) of PAL-NPs subunit nanovaccine immunization with 1M-Prime/IN-Boost route (FIG. 14A). For T cell responses, the single-cell suspension of harvested lungs was restimulated with overlapped spike peptide pools for 6 h and stained with canonical T cell markers and further analyzed with flow cytometry. Memory CD4C and CD8⁺ T cells are more prominent in the local tissues and are non-circulating, known as tissue-resident memory T cells (TRM). Traditionally, adjuvants are more responsible for the induction of potent antigen- specific T cell responses in protein subunit vaccines. The gating strategy for lung T cells subset is shown in supplementary FIG. 26A. We observed a significantly highest expression of CD4⁺CD69⁺ T cells (˜4.96 fold) in the lung of PUUC+CpG PAL-NP vaccine immunized mice compared to PBS, which is also highest among all other PAL-NP groups, as shown in FIGS. 148 and 14C, with flow cytometry plots (FCM) and the cell percentage. The CD69 is the earliest activation marker generated on the surfaces of antigen-specific activated lymphocytes. The PUUC PAL-NP vaccine formulation also significantly increases the expression of CD4⁺CD69⁺ T cell population (˜2.95 fold)(FIG. 14C). Interestingly, a significant amount of CD4⁺ T cells co- expresses both CD69⁺ and CD103Y markers, when mice were vaccinated with PUUC+CpG PAL- NP vaccine formulation (˜2.4 fold), which indicates the presence of lung CD4⁺ TRM responses (FIG. 14D).

Similar to CD4⁺ cell responses, we further analyzed the CD8⁺ T cell responses after PAL- NP vaccine immunization. We observed that lung CD8⁺ T cells significantly expressed substantially higher levels of CD69⁺ activation marker (˜2.15-fold), [FIGS. 14E and 14F (FCM plots and cell percentage)]and CD69⁺CD103⁺ tissue-resident memory markers (˜1.4 fold), [FIGS. 14E and 14G (FCM plots and cell percentage)], after PUUC+CpG PAL-NP vaccine immunization. We did not observe the significant population of effector memory CD4^{CD}44⁺ T cells (FIGS. 21A and 21E), but further gating shows the significant expression of CD4⁺CD44^{CD}69⁺ and CD4⁺CD44⁺CD69*CD103′ (resident memory) cell population with both PUUC+CpG and PUUC PAL-NP vaccine group and comparatively higher in PUUC+CpG PAL-NP group (FIG. 141H, 141, and 14J). This population represents the effector resident memory T cell population (EffTRM). We also evaluated the TRM cell population from total T cells (CD3⁺), which is significantly higher with the PUUC+CpG PAL-NP vaccine formulation and correlates with high frequencies of individual CD4 and CD8⁺ TRM cell population (FIGS. 20A and 20B). With the same formulation, a double negative CD4-CD8- population was also observed in T cell subset, which can be γδ T cells, probably activated in an MHC-independent manner and found in epithelial and mucosal tissues (FIG. 21K). This result shows that PUUC+CpG PAL-NP vaccine formulation induces strong local antiviral T cell immunity when administered IM-prime and IN-boost in mice.

The presence of anti-viral memory B cell responses in the local tissues also contribute to long-term immune protection against SARS-CoV-2. Therefore, we examined induced SARS-CoV-2 specific B cell responses with adjuvanated PAL-NP vaccine formulations via IM- Prime/IN-Boost immunization. After lung harvesting, single-cell suspension was stained with different B cell markers: antigen (RBD) specific, antibody-secreting cells (ASC), GC B cells, and memory B cells (tissue-resident memory B cells: BRM), and analyzed with the flow cytometry. The gating strategy for lung B cells is shown in supplementary FIG. 26B. Mice immunized with PUUC+CpG PAL-NP vaccine formulation through IM-Prime/IN-Boost route show a significant RBD tetramer B cell population [FIGS. 14K and 14L (FCM plots and percentage)], which is specific for receptor binding domain (RBD) of the spike protein. Compared to PBS control, the RBD tetramer+B cells are two-fold higher in mice immunized with PUUC+CpG PAL-NP vaccine formulation. In addition, upon IM-Prime/IN-Boost administration of PUUC+CpG PAL-NP vaccine formulation, we have observed the presence (non-significant) of several types of immune cells, including antibody-secreting cells (ASC: B220CD138′), IgG⁺ antibody-secreting cells (IgG⁺ ASC: B220CD138‘IgA’), gM memory B cells (B220‘IgM’IgD′CD38′), and IgG⁺ BRM (B220‘IgD-IgM’CD38′1gA-), as compared to control groups and other adjuvant groups (FIGS. 22A, 22C, 22E, 22G, and 22H). Interestingly, R848+CpG PAL-NP vaccine immunized mice with IM- Prime/IN-Boost strategy show a non-significant IgA′ ASC (IgA′ASC: B220′″CD138‘IgA’) and IgA′ BRM (B220′IgD-IgM-CD38′IgA⁴) cell population (FIGS. 22B and 22D).

(4) PUUC+CpG and PUUC PAL-NP Subunit Vaccine Formulation Enhances TH Type Immunity and Reduces the Secretion of TH2 Type Cytokines, when Delivered IM-Prime/IN-Boost

To further investigate the TH1/TH2 expression profile of the T cell population, restimulated lung cells from spike peptide pool were stained with intraccilular cytokines (Tumor necrosis factor-alpha: TNF-α, Interferon-gamma: IFN-γ, and Granzyme B: GrzB) and analyzed with flow cytometry (FIG. 15A). Mice immunized with PUUC+CpG PAL-NP vaccine formulation significantly increased the frequency of monofunctional CD4⁺ TRM which are enriched for THi type cytokine TNF-α (FIG. 15B and 15C). PUIUC PAL-NP vaccine formulation also shows a significant increase in the frequency of monofunctional CD4⁺ TRM population in lungs that express TH1 type cytokine IFN- (FIG. 15D and 15E). Similar to CD4⁺ TRM, the induction of CD8⁺ TRM cell population expressed TNF-α, is also enhanced by PUUC+CpG PAL-NP immunization (FIG. 15F and 15G). Lungs from mice immunized with PUUC+CpG PAL-NP vaccine formulation had a non- significant IFN-γ enriched CD8⁺ TRM cell population (FIGS. 1511 and 151). The PUUC PAL-NP group enhances the frequency of CD4⁺CD44⁺ TRM cell population that expresses GrAB (p-=0.0564) (FIGS. 15J and 15K). The PUUC PAL-NP vaccine formulation alone, also increases the percentage of GrzB expressing monofunctional CD4⁺CD44⁺ TRM, and CD4⁺CD44⁺ T cell population in a non- significant manner [FIG. 15I and 15M, and FIG. 21D]). PUUC-CpG PAL-NP vaccine formulation significantly increases the percentage of GrzB expressing monofunctional CD8⁺CD44⁺ T cell population in lungs (FIG. 21H). A significant population of CD4 TRM cells enriched with GrzB was observed in the mice vaccinated with PUUC PAL-NPs formulation (FIG. 201). Whereas both PUUC+CpG and PUUC PAL-NP formulations did not increase a significant population of GrzB expressing CDW TRM cells in the mice lungs (FIG. 20M). Mice vaccinated with PUUC PAL-NP vaccine formulation have non-significant CD4⁺ TRM polyfunctional cells that co-express TNF-α, and IFN-γ (FIG. 21I). Similarly, a non-significant increase in the percentage of CD8 TRM polyfunctional cells that co-express both TNF-α and IFN-γ was observed in the lungs of mice vaccinated with PUUC+CpG PAL-NP formulation (FIG. 21J). PUUC+CpG and RS48+CpG PAL-NP vaccine formulations significantly increase the CD4 CD8^{, CD}4 CD44⁺, and CD8⁺CD44⁺cell populations in the mice lungs that express TNF-α (FIGS. 20F, 20J, 21B, and 21F). PUUC+CpG PAL-NP vaccine formulation significantly increased the CD3⁺ TRM that expresses TNF-α (FIG. 20C), whereas the PUUC PAL-NP group showed a non-significant increase in total CD3⁺ TRM that expresses IFN-γ and GrzB (FIG. 20) and 20E). PUUC+CpG PAL-NP group induces a significant increase in the GreB expressing monofunctional CD8⁺ TRM cell population, but PUUC PAL-NP group induces a non-significant increase in GrzB expressing monofunctional CD4⁺ TRM cell population (FIGS. 20G and 20K). None of the adjuvanted PAL-NP formulations increase the CD4⁺ IFN-γ T cell population in the mice lungs (FIGS. 20H and 20L). For a more comprehensive study of TH VTH2 cytokine profile, we performed a multiplexed cytokine assay to assess various cytokine concentrations from supernatants of lung T cells after restimulation. Secreted cytokine profile is associated more with the TH1 type response, where PUUC+CpG PAL-NP vaccinated mice secrete TH1 type cytokine TNF-α (FIG. 15O), and PUUC PAL-NP vaccinated mice secrete TH1 type cytokine IFN-γ (FIG. 15N). This profile is consistent with prior assessment by flow cytometry data, where TNF-α and IFN-γ expressing T cells were significantly higher in RUG-1 adjuvanated PAL-NPs (PUUC+CpG) than the cohorts immunized with other adjuvants and controls. Thus, the elevated level of TH1 type (TNF-α and IFN-γ) cytokine (FIG. 15N and 15O) and suppressed (IL-10, IL-4) (FIGS. 15P and 15Q) or very low detection level of TH2 type cytokine (IL- 13) profile were observed in PUUC-CpG PAL-NP vaccinated mice (FIG. 21L). These results demonstrate that RIG-I targeted PAL-NP combination greatly enhanced T cells' potency and THI biased response.

(5) PUUC+CpG PAL-NPs Subunit Vaccine Formulation Elicits Comparable SARS-CoV-2 Mucosal Humoral Immunity with IN-Prime/IN-Boost

PUUC+CpG PAL-NP vaccine formulation was found to be one of the highly promising candidates that shows strong mucosal humoral and local cellular and, systemic responses with IM- Prime/IN-Boost. As a result, we conducted a more comprehensive second in vivo study, which is more concentrated on immune responses specific to the prime-boost vaccine administration routes. PUUC+CpG PAL-NP vaccine formulation is administered in mice with three prime-boost strategies: (A) IM-Prime/IN-Boost, (B) IN-Prime/IM-Boost, and (C) IN-PrimeiN-Boost. Mice were immunized prime at day 0 and boosted at day 21 with PUUC+CpG PAL-NP vaccine formulation and sacrificed at day 35. BAL fluid and serum samples were collected to analyze generated mucosal and systemic humoral response (FIG. 16A). We first evaluated mucosal anti- spike S1 IgA, IgG, and neutralizing antibody responses generated in BAL fluid through these three prime-boost strategies (FIG. 16B to 16F). Consistent with the first in vivo study, the IM-Prime/IN- Boost route enhances mucosal and systemic antibody responses. Surprisingly, IN-Boosting with PUUC+CpG PAL-NP vaccine formulation after IN-Priming (IN-Prime/N-Boost) induces substantial and significant anti-spike IgA level (A450-A630=2.4) in BAL fluid (at 1:2 dilution)(FIG. 168). Interestingly, a similar level of strong nAbs were induced with both IM-PrimeIN-Boost and IN-Prime/IN-Boost routes (at 1:2 dilution) (FIG. 16D). However, the total IgG level in BAL fluid is significantly higher in IM-Prime/IN-Boost, and follow this order: IM-Prime/IN-Boost>IN-Prime/IM-Boost>IN-Prime/IN-Boost (FIG. 16C). The IM-Prime/IN-Boost and IN-Prime/IM- Boost groups show more TH.I type immunity (IgG2aiIgG 1 ratio>l), whereas the IN-Prime/IN- Boost group is close to IgG2a biased immunity (FIGS. 16E and 16F, and FIG. 23C). The systemic humoral immune responses generated through IM-Prime/IN-Boost route are consistent with the first in vivo study results, showing a high and significant level of total IgG and nAb levels (FIG. 16G, 16H, and 16I). The secreted total IgG (AUC value) levels in serum after the PUUC+CpG PAL-NP immunization in mice with three prime-boost strategies follow the order: IM-Prime/IN-Boost>IN- Prime/IM-Boost>IN-Prime/IN-Boost [FIGS. 16G and 16H (IgG dilution curve and AUC)]. Interestingly, both IM-Prime/IN-Boost and IN-Prime/iM-Boost groups show more TH1 type immunity, whereas IN-Prime/IN-Boost group is close to IgG2a biased immunity [FIGS. 16J and 16K (IgG1 dilution curve and AUC), and FIGS. 16L and 16M (IgG2a dilution curve and AUC)]. With IN-Prime/IN-Boost, a significant serum IgA level group was observed, but it is comparatively lower than IM-Prime/IN-Boost group and follows the order IM-Prime/IN-Boost >IN-Prime/IN-Boost>IN-Prime/IM-Boost [(FIG. 16N and 16O (IgA dilution curve and AUC)]. These results altogether indicate that the PUUC+CpG PAL-NP vaccine formulation generates more potent mucosal and systemic humoral responses with the IM-Prime/IN-Boost group. However, the induction of strong mucosal IgA (comparable to IM-Prime/IN-Boost) and nAb level (similar to IM-Prime/IN-Boost) in the BAL fluid with the IN-Prime/IN-Boost group make this PUUC+CpG PAL-NP vaccine formulation also suitable for fully mucosal SARS-CoV-2 vaccine.

(6) PUUC+CpG PAL-NP Subunit Vaccine Elicits Robust SARS-CoV-2 T Cell (Tissue-Resident Memory) Immunity with IN-Prime/IN-Boost and B Cell Responses with IM-Prime/IN-Boost

In the second in vivo study, we further investigate route-specific cellular immune responses (T cell and B cell) with three prime-boost strategies: (A) IM-Prime/IN-Boost, (B) IN-Prime/AM-Boost, and (C) IN-Prime/IN-Boost)using PUUC+CpG PAL-NPs subunit vaccine formulation. For T cell responses, the lung single-cell suspension of harvested lungs was restimulated with spike peptide pools for 6 h, stained with canonical T cell markers, and further analyzed with flow cytometry (FIG. 17A). Gating strategy for lung T cells is shown in supplementary FIG. 26A. PUUC+CpG PAL-NPs group induced stronger and enhanced local T cell responses when delivered IN-Prime/IN-Boost, which are surprisingly higher than IM-Prime/IN-Boost. Analyzing tissue-resident memory T cells (TRM), we observed that a proportion of CD4 helper T cells significantly express both CD69⁺ and CD69⁺CD103 (CD4⁺ TRM) cells with IM-Prime/IN-Boost and IN-Prime/IN-Boost group, but the population frequency is higher in the later group [FIGS. 17B and 17C (FCM plots and CD4⁺CD69⁺ T cell percentage), and FIG. 17D (CD4⁺ TRM percentage)]. The frequency of CD4⁺CD69⁺ cells is also higher in IN-Prime/IN-Boost group (˜2.73 fold) than 1M- Prime/IN-Boost group (˜2.28 fold) with respect to the PBS control. Similarly, the frequency of CD4⁺CD69⁺CD103* population is slightly higher in IN-Prime/IN-Boost group (˜2.95 fold) than IM-Prime/IN-Boost group (˜2.92 fold). Overall, the frequency of generated CD4CCD69⁺ T cell population follows the order: IN-Prime/IN-Boost>IM-Prime/IN-Boost>IN-Prime/IM-Boost. In comparison, the CD4CD69″CD103* T cell population frequency follows the order: IN-Prime/IN-Prime>IM-Prime/IN-Boost>IN-Prime/IM-Boost. 170. With CD8⁺ T cells responses, we observed similar results. The frequency of CD8*CD69⁺ T cells is higher in the mice immunized through IN-Prime/IN-Boost strategy (˜2.37 fold) compared to IM-Prime/IN-Boost (˜2.29 fold) [FIGS. 17E and 17F (FCM plots and percentage)]. The frequency of CD8⁺ TRM in the IN-Prime/IN-Boost group (˜0.43 fold) is close to IM-Prime/IN- Boost group (˜0.4 fold) [FIGS. 17E and 17G (FCM plots and percentage)]. We also found that a proportion of CD4⁺CD44⁺ cells shows both CD69⁺ and CD69⁺CD103 populations, with both IN- Prime/IN-Boost and IM-Prime/IN-Boost groups, which indicate the presence of effector memory resident cell population (Eff TRM) (FIG. 17H and 171). However, the frequency of CD4*CD44⁺CD69⁺ cells are significantly higher in the IN-Prime/IN-Boost group, and the frequency of CD4^{CD}44⁺CD69″CD103′ cells is almost similar in both IN-Prime/IN-Boost and IM-Prime/IN- Boost groups. Like the CD4⁺CD44⁺ T cell response, we analyzed the CD8⁺CD44⁺ T cell responses after immunization, and similar results were observed. The frequencies of CD8*CD44⁺CD69⁺ and CD8⁺CD44*CD69^{CD}103⁺ T cells were also comparatively higher in IN-Prime/IN-Boost than IM-Prime/IN-Boost group. (FIG. 17J and 17K). We also evaluated the TRM cell population from total T cells (CD3⁺), which is higher with the IN-Prime/IN-Boost route and correlates with the frequencies of individual CD4⁺ and CD8⁺ TRM cell populations (FIG. 23A and 23B). Further confirmation of γδ phenotype from the first in vivo study, the lung cells were stained with TCRγδ: marker in the second in vivo study. We observed a high CD3″CD4-CD8-TCRγδ* cell population with IN-Prime/IN-Boost group (FIG. 23N). These results show that IN-Prime/IN-Boost administration of PUUC-CpG PAL-NPs, vaccine formulation induces strong antiviral T cell immunity in the lungs.

In addition to antiviral memory T cell responses, B cell responses are also necessary for clearing mucosal pathogens. Therefore, in the second in vivo study, we examined B cell responses generated with three prime-boost strategies (A) IM-Prime/IN-Boost, (B) IN-Prime/IM-Boost, and (C) IN-Prime/IN-Boost) using PUUC+CpG PAL-NPs vaccine formulation (gating strategy: supplementary FIG. 26B). We observed a significantly higher RBD tetramer B cell population with IM-Prime/IN-Boost, compared to IN-Prime/IM-Boost and IN-Prime/IN-Boost groups, which is specific for receptor binding domain (RBD) of spike protein [FIGS. 17L and 17M (FCM plots and percentage)]. At the same time, a non-significant increment in IgA antibody- secreting cells (IgA′ASC: B220′⁺CD138⁺IgA⁺) was observed with IM-Prime/IN-Boost route (FIG. 25B). The presence of IgA tissue-resident memory B cells (IgA^{BRM: B}220′IgD-IgMCD38⁺IgA⁺), GC-B cells (B220^{CD}38′GL7′), and IgM Memory B cells (B220˜IgDIgMCD38⁺) was also observed in a non-significant manner with IN-Prime/IN-Boost group (FIGS. 25E, 25D, and 25C).

(7) PUUC+CpG PAL-NPs Subunit Vaccine Formulation Enhances TH1 Type Immunity with IN- Prime/IN-Boost Group And Reduces the Secretion of TH2 Type Cytokines

In the second in vivo study, we further examine TH 1/TH2 expression profile by administrating the PUUC+CpG PAL-NP vaccine formulation in mice with three prime-boost strategies: (A) IM-Prime/IN-Boost, (B) IN-Prime/IM-Boost, and (C) IN-Prime/IN-Boost (FIG. 18A). After harvesting of lungs, the restimulated T cells from spike peptide pool, was further stained with intracellular cytokines: TNF-α, IFN-γ, and GrzB, and analyzed with flow cytometry. With both IN-Prime/IN-Boost and 1M-Prime/IN-Boost groups, we observed a significant monofunctional CD4C TRM population that expresses TH1 type intracellular cytokines: TNF-α, IFN-γ and cytotoxic GrzB [FIG. 18B, 18C, and 18D (FCM plots), and FIG. 18E (percentage)]. Similarly, with both the IN-Prime/IN-Boost and IM-Prime/IN-Boost groups, we observed a significant monofunctional CD8* TRM population that expresses TH1 type intracellular cytokines: TNF-α, IFN- γ, and cytotoxic GrzB [(FIG. 18F, 18G, and 18H (FCM plots), and FIG. 181 (percentage)]. We also observed the higher polyfunctional CD4⁺ TRM cell population, which co-express TH1 type cytokines: TNF- α and GrzB (FIG. 18J), IFN-γ and GrzB (FIG. 18K), and TNF-α and IFN-γ (FIG. 230), with IN- Prime/IN-Boost group compare to IM-Prime/IN-Boost group. Similar results were observed with the CD8⁺ TRM cell population with PUUC+CpG PAL-NP vaccine formulation when immunized IN-Prime/In-Boost (FIGS. 18t and 18M and, FIG. 23P). Furthermore, the monofunctional and polyfunctional CD4⁺ TRM and CD8⁺ TRM cells are significantly higher in IN-Prime/IN-Boost group than IM-Prime/IN-Boost group. The IN-Prime/IN-Boost group also induces CD3⁺ TRM cell populations that express TNF-α, IFN-γ, and GrzB (FIGS. 23E, 23F, and 23G). We also observed significantly high monofunctional CD4⁺, CD4⁺CD44⁺, CD8⁺*CD44⁺, and CD8⁺ T cell populations enriched for GrzB, with IN-Prime/IN-Boost group (FIGS. 23H, 23K,24B, and FIG. 24F). 173.

The IN-Prime/IM-Boost and IN-Prime/IN-Boost groups non-significantly increase the monofunctional CD4⁺. CD4⁺CD44, CD8 CD44⁺, and CD8⁺ T cell populations enriched for TNF-α (FIGS. 23J, 23M, 24D, and FIG. 24H). There is a non-significant increase in the IFN-γ enriched CD8⁺CD44⁺ cell population with IN-Prime/IN-Boost and IN-Prime/IM-Boost groups (FIG. 24G). At the same time, the IN-Prime/IN-Boost group shows a significant increase in the frequency of monofunctional CD4⁺CD44⁺ TRM and CD8⁺CD44⁺ TRM cell population that expresses TH1 type cytokines: TNF-α, IFN-γ, and cytotoxic GrzB, compared to IM-Prime/IN-Boost groups (FIGS. 24I and 24J). For a more comprehensive study of secreted TH1/TH2 cytokine profile, the supernatants of restimulated lung cells were analyzed with multiplexed cytokine assay to assess various cytokine concentrations (FIG. 25F to 25K). Secreted cytokine profile is more associated with TH1 type response with both IM-Prime/IN-Boost, and IN-Prime/IM-Boost groups, which secretes TNF-α and IFN-γ cytokines, respectively. A very low level (or below the detection limit) of TH2 cytokines (IL-4, IL-5, IL-13, IL-10) were observed with IN-Prime/IN-Boost group. These results demonstrate the enhanced and potent TH1 type response in T cells, elicited through IN-Prime/IN- Boost group with PUUC+CpG PAL-NP vaccine formulation.

### b) Discussion

The continuous waning of pre-existing systemic immunity and immunovasion reduces the current SARS-CoV-2 vaccine's efficacy for preventing viral transmission. To combat this, vaccines that target the mucosal tissues and induce robust and balanced mucosal-systemic immunity are required to reduce viral shedding, prevent initial infection, and provide overall protection. Protein subunit nanovaccines formulated with an appropriate combination of adjuvants can represent a promising strategy for developing mucosal vaccines. Moreover, other specific factors such as nanovaccine design, immunization methods (different prime-boost regimen), respective vaccine doses, and booster time intervals also play a significant role in modulating the SARS-CoV-2 immune response. Therefore in this study, we focus on three important aspects of developing a potent SARS-CoV-2 mucosal subunit nanovaccine: i) designing the nanoparticle by functional group modification in biopolymer that enhance adjuvant delivery and immunomodulation (ii) screening multiple adjuvant combinations of RLRs and TLRs agonists on nanoparticles for enhanced SARS-CoV-2 mucosal immune responses, and (iii) vaccine administration route-specific (prime and boost) comparative study using screened combination adjuvants NPs.

Proper design and synthesis of polymeric biomaterial-based nanocarriers are essential for the enhanced delivery of multiple combination adjuvants that modulate the immune response. Cationic polysaccharide biomaterials are commonly used for mucosal delivery due to their excellent mucoadhesive property and adjuvanticity. However, the high number of primary amines in polysaccharides can generate systemic toxicity, which can be reduced by chemical modification with higher-order amines (like secondary/tertiary). Therefore, we chose the polysaccharide as a base polymer for NP synthesis and performed the chemical/structural/functional modification to reduce toxicity and enhance multiple adjuvants loading/delivery capability on NPs. This results in an amphiphilic polymer known as polysaccharide-amino acid (arginine/histidine)-lipid polymer (PAL polymer), which undergoes the self-assembly process to form the PAL nanoparticles. Each chemical modification in the polysaccharide has its specific role: (a) the stearyl lipid core provides nanoparticle stability and better encapsulation of hydrophobic adjuvants (TLR7/8 agonist- R848), b) the cationic arginine/histidine aid in surface loading of anionic adjuvant (PUUC RNA: RIG-I agonist, CpG DNA: TLR9 agonist) and provide buffering capacity/endosomal escape for enhanced adjuvant delivery, c) the incorporation of disulfide bond maintains the NP degradability and enhances the adjuvant's delivery properties. This improved rational design of polymer and nanocarrier aids in achieving a balanced multiadjuvants delivery, demonstrating minimal toxicity in vitro and in vivo, and ultimately enhancing vaccine effectiveness.

Initial in vitro investigations indicate that adjuvanated PAL-NPs targeting RIG-I and TLRs in different combinations such as: PUUC, R848, PUUC+CpG, and R848+CpG, elicit more diverse proinflammatory cytokine responses (including IL12p70, IL-1β, and IFN-β) with GM-CSF differentiated BMDC, likely due to a broader population of APCs. Therefore, these adjuvanated PAL-NP groups can also induce more robust humoral and cellular responses in vivo.

Thus, we designed an in vivo SARS-CoV-2 vaccination study using adjuvanted PAL-NP formulations in a mouse model and conducted two distinct experiments. To conduct in vivo studies, the PAL nanovaccine formulation was prepared by mixing recombinant and stabilized S1 trimer subunit of the SARS-CoV-2 pathogen with multiadjuvanated PAL-NPs. For the physiological resemblance of multiadjuvanated PAL-NPs with the SARS-CoV-2 pathogen (containing ssRNA), a RIG-I agonist PUUC (ssRNA) is selected as one of the major adjuvants. Cytosolic RIG-I-like receptors recognize PUUC RNA and activate them to induce potent antiviral immunity. In the first in vivo study, we thoroughly investigate the combination adjuvant-mediated immunomodulation using four adjuvanated (R848, PUUC, R848+CpG, PUUC+CpG) PAL nanovaccine formulations, after administrating them in mice with IM-Prime/IN-Boost strategy. This study helps to choose the best multiadjuvants PAL nanovaccine candidate, which strengthens the existing IM immunity and triggers the potent mucosal immune responses against SARS-CoV-2. In the second in vivo study, we conducted a comprehensive head-to-head comparative study of modulated immune responses with different prime-boost immunization routes using the best multiadjuvants nanovaccine candidate examined from the first in vivo study.

Results from the first in vivo study revealed that the PUUC+CpG PAL-NPs is the best Mult adjuvanted nanovaccine, as this formulation elicits strong mucosal humoral (IgA and IgG), systemic humoral (IgG and nAb), and local cellular responses. Mucosal IgA is the major antibody response that restricts the entry of respiratory viral pathogens (like influenza, SARS-CoV-2, and MERS CoV) and is the most dominating antibody during early immune response after infection, thus providing sterilizing immunity. The PUUC+CpG PAL-NPs group also induced TH 1 polarized antibody response (IgG2a switching) in both BAL fluid and serum. Prior research has also shown comparable outcomes with adjuvant-based influenza vaccines containing RIG-I agonists that enhance the induction of IgG2a subclass response. Despite being a preclinical study on mice, our present work can be correlated to human humoral responses, especially the human IgG1, which is analogous to mice IgG2a isotypes and is the most preferred subclass of IgG antibody exhibits optimum antiviral activity. These findings further highlight the importance of RIG-4 agonists in fighting respiratory viral diseases. IN-boosted, PUUC+CpG PAL-NPs after IM priming are the potent inducer of local T cell responses (CD4⁺, CD8, and CD4⁺CD44⁺ TRM) compared to other adjuvant combinations except for PUUC PAL-NPs, which also induces a significant CD4⁺ TRM population. The CD8⁺ tissue-resident memory T (TRM) cells are known to be more effective for viral clearance, and CD4C TRM is involved in a broad spectrum of activities, including the durability of neutralizing antibody responses and promoting the development of protective memory B cells.

Studies on some adjuvanated vaccines (alum and CpG adjuvanated) for RSV and SARS-CoV have shown that vaccine-associated enhanced respiratory disease (VAERD) in patients has a connection with CD4 TH2 type response. However, our results show that the RIG-1 targeted PUUC+CpG and PUUC PAL-NP group induces TRM, that expresses TH1 type cytokines, and similar results were observed with secreted cytokine expression profile which shows an elevated level of TH1 type cytokine with suppressed or very low detection level of TH2 type cytokines. PUUC+CpG PAL-NPs group also elicits CD8⁺CD44⁺ cells that express higher cytotoxic molecules, like GrzB, and with PUUC PAL-NP group, the induced CD4⁺CD44⁺ cell populations express GrzB. However, CD8⁺ cytotoxic T cells are classically associated with virus- infected cell killing, and CD4⁺ GrzB cytotoxic T cells can be a significant part of the human antiviral T cell responses. Therefore, both the PUUC+CpG and PUUC PAL-NPs groups, which share a RIG-1 agonist as a common adjuvant. significantly enhance the magnitude of TRM responses, polarizing it to TH1 profile, and lead to potent antiviral immunity without showing pathogenic TH2 type responses. Induction of antigen-specific RBD tetramer⁺B cells with PUUC+CpG PAL-NP group signifies the antigen encounter and further B cell activation and formation of memory B cells. PUUC+CpG PAL-NP group also enhances the induction of IgM BRM, IgG⁺ BRM, and IgG⁺ASC. During reinfection, BRM cells are known to produce rapid and immediate recall responses against pathogen entry at mucosal tissues.

Although adjuvants can play a crucial role in enhancing potent antiviral mucosal immunity, most studies investigating their effectiveness have focused on I M vaccines with limited knowledge about the role of adjuvants in mucosal vaccines. For example, in humans, CpG (TLR9 agonist) based subunit vaccines elicit a systemic immune response when administered IM and are not an ideal adjuvant candidate for IN immunization. Few recent studies focused on RIG-I and TLRs targeted SARS-CoV-2 protein-subunit vaccines that can provide a useful comparison point for our study. Nguyen et al. developed a C.S./CpG/R2BD intranasal vaccine that induces mucosal humoral response and has demonstrated efficacy against VOCs but lacks to generate local T cell immunity. Jangra et al. presented a preclinical study showing that a three-dose nanoemulsion R.IG-I agonist (IVT DI) adjuvanated SARS-CoV-2 subunit IN vaccine results in systemic TH 1 and only IgG responses in both BAL fluid and serum. Despite using multiple high antigen doses of Sl subunit protein and incorporating R.IG-I agonists, the study did not observe an enhanced lung-specific humoral and memory T cell response. The aim of using mucosal vaccines is to establish protective local antiviral immunity, While preclinical studies have shown some success in achieving this, they have not fully met the criteria for overall protection. Therefore, it is essential to enhance current SARS-CoV-2 vaccine strategies by incorporating combination adjuvants.

In contrast, a recent preclinical study by Tianyang et al. showed that IM priming of m- RNA LNP and IN-Boost spike protein only (unadjuvanated) elicits protective SARS-CoV-2 immunity. In our study, we studied how adjuvants, specifically combination adjuvants induce or improve mucosal and systemic immunity. We showed that multiadjuvanated PUUC+CpG PAL- NP group offers an elevated level of mucosal antiviral immunity, both humoral and cellular, as well as a systemic humoral immune response with the IM-Prime/IN-Boost strategy. Also, robust and broad local T cell and comparable mucosal humoral (IgA and nAb) immune responses are induced with 1N-Prime/IN-Boost. In comparison to the unadjuvanted approach, our results have revealed significant benefits of using adjuvants. RIG-I is triggered by the native SARS-CoV-2 virus, so we hypothesized that it can mimic infection. Other reported adjuvant formulations, such as BECC: TLR4 agonist and cationic liposome (CAF01), also enhanced mucosal immunity, but with limited local T cell responses. The recently approved mucosal vaccines by India, China, and others in clinical trials appear to prioritize the induction of a mucosal humoral immune response as indicated by immune markers such as IgA, IgG, and nAbs. However, the development of robust resident memory T cell responses in the nasal-associated lymphoid tissue (NALT) and lungs provide a more favorable immune signature for long-term immunity and better control of lung infections which can lead to stronger protection compared to circulating cellular responses. Our study also confirmed that the IM-Primed/IN-Boosted PUUC+CpG PAL-NP group induces high-quality, robust mucosal and systemic antibody responses, potent cellular response, and heavily favoring TH 1 responses, making it more suitable for a mucosal subunit vaccine.

Different vaccine administration routes have different mechanisms to induce an immune response. We also confirmed this with our second in vivo study results, where PUUC+CpG PAL nanovaccine formulation was administered in mice via three different prime-boost routes: IM-Prime/IN-Boost, IN-Prime/IN-Boost, IN-Prime/IM-Boost. Our study found that IN-Boosting effectively and significantly enhanced the IM-prime generated immunity (IM-Prime/IN-Boost) with muhiadjuvant PUUC+CpG PAL-NP group, which is consistent with our first in vivo study and recent studies available in the literature. Interestingly, after IN priming, the IN- Boosted mice generated a higher level of lung T cell immunity than IM-Prime/IN-Boost group, along with good local humoral responses (IgA and nAb). The induction of lower T cell response with IN-Prime/7M-Boost vaccination indicates that IM-Boost did not enhance the T cell immunity generated by IN-prime, but rather, the overall response was significantly elevated with IN-Boost as observed in IN-Prime/IN-Boost group. This finding is an interesting point to consider.

Along with a strong T cell response and a comparable mucosal humoral response, the IN- Prime/IN-Boost group shows a considerable systemic IgG response but a relatively lower systemic nAb response than the other two groups, which include IM vaccination in either the prime or boost. The IN-Prime/IN-Boost group enhances the production of monofunctional and polyfunctional subsets of TRM cells (CD4⁺ and CD8⁺) that express TH 1 type intracellular cytokines: TNF-α, IFN- γ, and GrzB, but not the pathogenic TH2 type. Their levels are higher than those seen in the IM- Prime/IN-Boost and IN-Prime/IM-Boost groups. A similar trend of T cell and cytokine data was observed in the study on recovered SARS-CoV-2 patients by Grifoni e/al., which showed that T cell responses appeared as TH1 phenotype with lower levels of TH2 type response.

Additionally, most IFN-γ CD8⁺ T cells co-expressed GrzB and TNF-α in the same study, which persists in our results also. The large population of polyfunctional T cells producing multiple T H1 cytokines demonstrates adequate antigen-presentation and strong co-stimulation from professional APCs. As SARS-CoV-2 continuously evolves with new immune evasive variants, the population needs to be boosted with the new-generation potent mucosal vaccines that provide enhanced protection and reduced transmission while maintaining their safety and efficacy. Although our results focused on only nanoparticle-based subunit vaccines, if used as a booster, we believe this design can broadly apply to other primary immunization methods. Our preclinical study proves that the mucosal (IN) route is essential for new vaccination strategies and should be included in the current immunization protocols.

In conclusion, multiadjuvanted PUUC+CpG PAL-NP based subunit mucosal vaccine induces robust and potent antiviral mucosal immunity against SARS-CoV-2. We developed an immunization strategy where a balanced mucosal-systemic immunity is induced by the PUUC+CpG nanovaccine when delivering parenteral prime and intranasal boost. Promising outcomes from the intranasal prime and boost nanovaccine delivery also suggest the possibility of a fully mucosal delivery route. These results ensure that this uniquely designed multiadjuvant nanoparticle has great use for SARS-CoV-2 mucosal subunit nanovaccines. Our results are highly promising in preclinical studies, but due to the inherent immunological differences between animal models and humans, it requires further optimization for future clinical and translational use.

### c) Materials and Methods

All animal experiments were conducted in accordance to approved IACUC (Institutional Animal Care and Use Committee) protocols by the Georgia Institute of Technology.

### (1) Synthesis of PAL-NPs and multiple adjuvant loading

Amphiphilic polysaccharide-amino acid-lipid polymer was synthesized and characterized as described in the supplementary information (see supplementary materials, FIG. 19A). Cationic polysaccharide-amino acid-lipid nanoparticles (PAL-NPs) are synthesized by probe sonication using an amphiphilic PAL polymer with a final concentration of 0.5 mg/ml. The polymer was first hydrated and dispersed overnight in phosphate buffer saline (PBS, pH 7.2, 10 mM). The hydrated polymer was mixed with DMSO (PBS: DMSO ratio, 80:20), and probe sonicated on ice for 10 min. Nanoparticles were purified with vigorous dialysis in PBS (pH 7.2, 10 mM) for one day by changing water thrice. R848 adjuvant encapsulated cationic PAL-NPs (0.5 μg R848 per mg) were synthesized by the addition of R848 stock in DMSO, followed by probe sonication and dialysis. Nanoparticles were concentrated accordingly to the volume required for the in vivo and in vitro studies. Nanoparticles were electrostatically loaded with nucleic acid adjuvants, either CpG ODN 2395 (Invitrogen, Cat #tlrl-2395) or PUUC in 10 mM sodium phosphate buffer (made with nuclease-free water) and left for rotation for 24 h (See Table I for adjuvant doses). All adjuvants and antigen stock (except R848) were prepared in nuclease-free water. PUUC RNA was synthesized and characterized. Characterization of adjuvant loading on nanoparticles was described in the supplementary information.

(2) In Vitro Activation of Mouse BM-APCs with Multiadjuvanatyed PAL-NP Formulations

GM-CSF-derived BMDCs were generated. At day 7 of the culture, mBMDCs derived from GM-CSF were seeded in 96-well plates at a density of 500,000 cells per well and allowed to settle for 2 h. Adjuvanated PAL-NP formulations (see Table I for adjuvant and PAL-NP doses) were then added to the wells. After treatment, the supernatants were collected 24 hours later, and cytokine concentrations (lL-lp, IFN-β, and IL12p70) were measured using ELISA assays.

### (3) In Vivo Vaccination Studies

BALB/c female mice (6-8 weeks old Jackson Labs, Bar Harbor, ME) were used to measure the adaptive immune response and anesthetized using 30% v/v Isoflurane diluted in propylene glycol for vaccination. Final nanovaccine formulations are prepared by mixing the adjuvanated PAL-NPs (250 μg per mice) and recombinant, stabilized SARS-CoV-2 spike SI trimer protein (1 μg/mice, SPN-C52H9, ACRO Biosystems). For intramuscular vaccination, formulations were prepared in total 100 μl of PBS (pH 7.2, 10 mM), out of which 50 μl was injected to the right and 50 μl to the left anterior tibialis muscle at day 0 as the first dose and at day 21 for boost doses). The doses of adjuvants on the PAL-NP adjuvants formulation for the IM vaccination (per mice) are PUUC (20 μg), CpG (40 μg), and R848 (20 μg), and also shown in Table 1. For intranasal vaccination, the formulations are prepared in a total of 40 μl of PBS (pH 7.2, 10 mM), out of which 20 μl was administered dropwise in both the left and right nares. The doses of adjuvants on the PAL-NP adjuvants formulation for the IN vaccination (per mice) are PUUC (20 μg), CpG (20 μg), R848 (20 μg), as shown in Table 1.

| | | | | Adjuvant dose | Adjuvants loading |
|---|---|---|---|---|---|
| PAL-NP | | Polydispersity | Zeta potential | for BMDCs | levels for in-vivo |
| Formulations | Size (nm)^{#} | Index (PDI)^{#} | (mV)^{#} | (ng/5e5 cells/mL) | studies (ug/mg NPs) |
| Blank PAL-NPs | 249.4 ± 31.1 (n = 3) | 0.21 | 31.7 ± 2.5 (n = 3) | 12 ug NPs | 250 ug NPs (IN and IM) |
| R848 PAL-NPs | 236.0 ± 65.2 (n = 3) | 0.175 | 30.0 ± 6.92 (n = 3) | 20 ng R848 | 80 ug R848/mg NPs |
| | | | | | (IN and IM) |
| CpG PAL-NPs | 249.4 ± 31.1 (n = 3) | 0.21 | 31.7 ± 2.5 (n = 3) | 100 ng CpG | 160 ug CpG/mg NPs- IM |
| | | | | | 80 ug CpG/mg NPs- IN |
| PUUC PAL-NPS | 249.4 ± 31.1 (n = 3) | 0.21 | 31.7 ± 2.5 (n = 3) | 100 ng PUUC | 80 ug PUUC/mg NPs |
| | | | | | (IN and IM) |
| R848 + CpG PAL-NPs | 236.0 ± 65.2 (n = 3) | 0.175 | 30.0 ± 6.92 (n = 3) | 20 ng R848 and | 80 ug R848 and 80 ug |
| | | | | 100 ng CpG | PUUC/mg NPs (IN and IM) |
| PUUC + CpG PAL-NPs | 249.4 ± 31.1 (n = 3) | 0.21 | 31.7 ± 2.5 (n = 3) | 100 ng CpG and | 160 ug CpG and 80 ug |
| | | | | 100 ng PUUC | PUUC/mg NPs- IM |
| | | | | | 80 ug CpG and 80 ug |
| | | | | | PUUC/mg NPs- IN |
| R848 + PUUC PAL- | 236.0 ± 65.2 (n = 3) | 0.175 | 30.0 ± 6.92 (n = 3) | 20 ng R848 and | — |
| NPs | | | | 100 ng PUUC | |
| R848 + CpG + PUUC | 236.0 ± 65.2 (n = 3) | 0.175 | 30.0 ± 6.92 (n = 3) | 20 ng R848, 100 ng | — |
| PAL-NPs | | | | CpG and, 100 ng | |
| | | | | PUUC | |

^{#}Size, PDI, and zeta potential measurements were taken for all NPs prior to electrostatically loading adjuvants PUUC or CpG.

### (4) Ex Vivo Lung Cell Restimulation and T Cell Staining

After harvesting the lungs from vaccinated mice (individual experiment), single-cell suspensions were prepared with a gentleMACS™ Octo Dissociator and Lung Dissociation Kit (Miltenyi Biotec) according to the manufacturer's instructions, including RBC lysis. Cells were centrifuged and resuspended at 10 million cells/mL in RPMI media with 10% FBS, 1% penicillin- streptomycin, I mM sodium pyruvate, and lx p-mercaptoethanol. Cells were seeded at 2 million cells per well in a U-bottom 96-well plate and left to culture overnight (at 37C with % CO2).

Lung cells were centrifuged and resuspended with fresh complete R.PMI media with 20 μL/mL of PeptTivator® SARS-CoV-2 Prot S (Miltenyi Biotec) and 5 μg/mL Brefeldin A (Biolegend). After incubation for 6 h, cells were stained for 30 min at RT with Zombie Green™ Fixable Viability Kit (Biolegend) and were blocked with anti-mouse CD16/32 (Biolegend) and True-Stain Monocyte Blocker™ (Biolegend). For blocking of cell surfaces, the cells were stained for 30 min at 4° C. with surface antibodies: anti-mouse CD3 (Biolegend, APC Fire 810), CD4 (Biolegend, APC), CD8a (Biolegend, PE/Cy5), CD44 (Biolegend, BV711), CD69 (Biolegend, BV785), CD103 (Biolegend, PE-Dazzle 594), CD56 (BD, BUV395), and TCR-y8 (Biolegend BV510). After surface staining, the cells were stained for intracellular cytokines. The cells were fixed and permeabilized for 30 min with the Foxp3/Transcription Factor Staining Buffer Set (eBioscience) at 4° C. Then cells were stained with anti-mouse TNF-α (Biolegend, PE/Cy7), IFN-γ (Biolegend, PE), and Granzyme B (Biolegend, Pacific Blue). Cell population data were acquired on Cytek Aurora flow cytometer and analyzed using FlowJo Software (FIG. 26A for the gating strategy).

### (5) B Cell Staining and Flow Cytometry

Lung single-cell suspensions were stained for 30 min at RT with Zombie Red™ Fixable Viability Kit (BioLegend) and were blocked with anti-mouse CD16/32 (Biolegend) and True-Stain Monocyte Blocker™ (Biolegend). Cells were washed once with PBS before surface staining. Following blocking, cell surfaces were stained for 30 min at 4° C. with anti-mouse GL7 (Biolegend, Pacific Blue), IgM (Biolegend, Pe-Cy7), CD138 (Biolegend, PerCP/Cy5.5), CD19 (Biolegend, AF700), IgA (SouthernBiotech, FITC), B220 (Biolegend, BV711), CD38 (Biolegend, APC Fire 750), and anti-IgD (Biolegend, BV605), PE-SARS-CoV-2 RBD tetramer, APC-SARS-CoV-2 RBD tetramer for 30 min at 4° C. (tetramer synthesis is described in supplementary information). After washing with PBS, cells were fixed using 4% paraformaldehyde. Cell population data were acquired on Cytek Aurora flow cytometer and analyzed using FlowJo Software (FIG. 26B for gating strategy).

### (6) ELISA Assay for Quantifying Anti-Spike Antibody Responses

The diluted recombinant SARS-CoV-2 Spike His Protein, CF (R&D Systems, Cat #11058-CV) (1 μg/mL in 0.05 M carbonate-bicarbonate buffer, pH 9.6) was coated onto Nunc™ MaxiSorpM ELISA plates by adding 100 ng/well and incubating the plates overnight at 4° C. Antigen-coated plates were washed three times with PBST wash buffer (prepared by mixing 10 mM PBS and 0.05% Tween-20), and plates were blocked for six hours at 4° C. with PBSTBA (prepared by mixing PBST with 1% BSA and 0.02% NaN3). Blocked plates were incubated overnight at 4° C. with diluted serum and BAL fluid samples (individual experiments). Plates were washed three times with PBST. A secondary biotinylated anti-mouse IgA, total IgG. IgG1, or IgG2a antibody (SouthernBiotech) which is 5,000-fold diluted in 5-fold diluted PBSTBA, was added to plates for 2 h at RT. Plates were similarly washed with PBST. After two hours, a 5,000- fold diluted streptavidin-conjugated horseradish peroxidase (strep-HRP, ThermoFisher) was added to the plates and incubated for the next 2 h at RT. The plates were again washed six times. Ultra TMB-ELISA Substrate Solution (ThermoFisher) was incubated for 15 to 20 min for color development on the plate. Lastly, stop solution (2 N sulfuric acid) was added to each well, and absorbance was measured at 450 and 630 nm (background) on a Synergy H.T. plate reader (BioTek) with Gen5 software.

### (7) Modified ELISA Assay to Measure Anti-Spike Neutralizing Antibodies

For the quantification of neutralizing antibodies, the above-described ELISA method is used with slight modification. A diluted recombinant SARS-CoV-2 Spike His Protein, CF (R&D Systems, Cat #11058-CV) in 0.05 M carbonate-bicarbonate buffer (1 μg spike/mL, pH 9.6) was incubated in wells of a Nunc^{y}M MaxiSorp™ ELISA 96-well plate (100 ng/well) overnight at 4° C. Plates were washed three times with PBS-Tween wash buffer (PBST). Antigen-coated plates were blocked in PBSTBA for six hours at 4° C. Blocked plates were again incubated overnight with serum and BAL fluid samples (individual experiments). Plates were similarly washed. Plates were incubated with PBSTBA diluted 500 ng/mL (25 ng/well) recombinant biotinylated human ACE-2 (R&D Systems, Cat #BT933-020) for 2 h at RT. The plates were washed again with PBST. A 5,000-fold diluted strep-HRP (ThermoFisher) was added to the plates and incubated for 2 h at RT. Plates were extensively washed six times and incubated with the optimized volume of Ultra TMB- ELISA Substrate Solution (ThermoFisher) for 20 min. In the end, the reaction was stopped with a stopping reagent (2 N sulfuric acid), and absorbance was measured at 450 and 630 nm (background) on a Synergy H.T. plate reader (BioTek) with GenS software.

### (8) Statistical Analysis

All flow cytometry FCS files were analyzed with FlowJo (v0, B.D.). Statistical analyses were performed with GraphPad Prism 9. For comparisons of more than two groups, statistical differences between normally distributed datasets were determined with a one-way analysis of variance (ANOVA) followed by Tukey's and Bonferroni's post hoc test for multiple comparisons. Similarly, nonparametric datasets were evaluated with the Kruskal-Wallis test and Dunn's post- hoc test. P<0.05 was considered statistically significant.

### (9) Materials:

Chitosan polysaccharide (Mw 15KDa) was purchased from Polysciences (85% degree of deacetylation). Dialysis tubing (MWCO 3.5 kDa, 1OkDa) was purchased from Thermo-Fisher Scientific. NMR solvents and other solvents for synthesis, such as ethanol and diethyl ether, were purchased from Sigma Aldrich.

### (10) Synthetic Steps for Polysaccharide (Chitosan)-Amino Acid-Lipid Polymer: Multistep Synthesis

### (a) Synthesis of O-Carboxymethyl-Chitosan

OCMC O-Carboxymethyl-Chitosan (OCMC) was synthesized to increase the selective O-carboxylation and reduce the N-carboxylation. The C-6 position of chitosan polysaccharide (500 mg) was first alkalized with 50% aqueous NaOH (20 mL) at −10° C. for one hour. The alkalized polysaccharide was further reacted with 2.5 g monochloroacetic acid (Sigma Aldrich) at 45-55° C. for six h. The reaction mixture was added with 70% ethanol to prepare the sodium salt of OCMC, which was further purified by vacuum filtration. The OCMC sodium salt was washed with 70% ethanol and acidified with 1 N HCl to form OCMC. The obtained OCMC was filtered and dried under a vacuum for further use. The incorporation of O-carboxymethyl group at the C-6 position was confirmed by 1HNMR (FIG. 27).

### (b) Synthesis of Thiolated O-Carboxymethyl-Chitosan

OCMC-SH Thiolated OCMC was synthesized. Briefly, synthesized OCMC was thiolated by covalent conjugation of carboxyl of thioglycolic acid with the amine group of chitosan (C-2 position) using carbodiimide chemistry. Firstly, the carboxyl group of TGA (500 mg, Sigma Aldrich) groups was activated with 1-ethyl-3-(3- dimethylaminopropyl) carbodiimide hydrochloride (EDC, Thermo Fischer) at pH 6.5 in DI water with a final concentration of 125 mM for 2 h. 250 mg of OCMC was acidified with 1M HCi. OCMC solution was added to the activated TGA solution, and the pH of the reaction medium was adjusted to 5 to avoid forming the disulfide bond. To eliminate excess TGA and to purify the thiolated OCMC, the reaction mixtures were dialyzed five times using dialysis membrane 10 kDa M WCO (Sigma Aldrich) for two days in the dark against HCl (5 mM), then two times against HCl (5 mM) with 1% NaCl at 10° C., which helps quench the ionic interactions between anionic sulfhydryl and the cationic polymer. Final dialysis was performed against 1 mM HCl to maintain the pH of the thiolated OCMC polymer to 4. Polymers were further lyophilized and stored at 4C until further use. Thiolation was confirmed by using 1HNMR (FIG. 28) and Elmann's assay (FIG. 19B).

### (c) Synthesis of Cysteamine Conjugated OCMC

OCMC-S-S-Cys The above lyophilized thiolated OCMC was first reduced with DTT (Dithiothreitol, Sigma Aldrich) before cysteamine conjugation. The necessary reduction step reduces the disulfide bond formed during lyophilization and helps increment free sulfhydryl groups. For reduction, the thiolated OCMC-SH solution was prepared in DI water, and pH was maintained at 8 using 1 M NaOH. DTT (Dithiothreitol, Sigma Aldrich) was added in a final concentration of 100 mM, and the reaction mixture was continuously stirred at RT. After approximately two h, NaCl was added to the reaction mixture (final concentration 1%, weight/volume), and the pH was adjusted to 4 with 1 M HCi. The resulting solution was dialyzed using a similar method as discussed in step 2.1.2. Finally, the solution was filtered through a 0.5 μm filter. The second cycle with DTT was repeated to improve the reduction of remaining disulfide bonds. The filtered thiolated OCMC was conjugated to cysteamine by forming a disulfide bond between thiols of cysteamine (Sigma Aldrich) and free thiols on OCMC. Cysteamine solution was prepared in 1% acetic acid, and the solution pH was adjusted to 6.0. The thiolated OCMC was added dropwise over a three h period using an addition funnel to the cysteamine solution. The reaction mixture was allowed to stir for 24 h at RT, and the pH of the final solution was maintained between 4-5. The resulting polymer conjugates were isolated using a similar dialyzing method, as discussed in step 2.1.2. Polymers were further lyophilized and stored at 4° C. until further use. Synthesis of OCMC-S-S-Cys was confirmed by using 1 HNMR (FIG. 29) and Elmann's assay (FIG. 19B). The concentration of thiols was decreased after disulfide formation, as shown by Elmann's assay, which confirms the synthesis of OCMC-S-S-Cys formation.

### (d) Synthesis of arginine and hitddine conjugated OCMC,S-S-Cysteamine

OCMC-S-S-(A/H) The coupling of both amino acids: N-Boc Histidine (Alfa Aesar) and N-Boc Arginine (Alfa Aesar), onto cysteamine conjugated OCMC was performed by the reaction of the amine groups of OCMC-S-S-Cys and carboxylic group of amino acid in the presence of coupling agents EDC (Thermo Fischer Scientific) and NHS (Sigma Aldrich). The Boc-protected amino acids were used to reduce the cross-reaction of the free carboxyl group of the C-6 position of chitosan with the amine groups of amino acids. The free carboxyl group of N-Boc Histidine (0, 5, 10 mM) and N-Boc Arginine (0, 10, 20 mM) was first activated individually by the addition of EDC/NHS (10 molar excess) in TEMED/HCL buffer (I % concentration, v/v) at pH 5.5 (Tetramethylethylenediamine, Sigma Aldrich) for 2 h at 25° C. The activated amino acid solution was added dropwise to the solution of OCMC-S-S-Cys in the same buffer and reacted for the next 16 h. The concentration of both amino acids was used with different ratios to yield conjugates with different degrees of substitution. The pH of the final reaction mixture was maintained at 6. Then the reaction product was extensively dialyzed against distilled water for two days, and the pH was maintained at 6.5 to remove the unreacted components. The purified polymer was then recovered by lyophilization and stored at 4° C. The incorporation of arginine and histidine in the polymer chain was confirmed by 1 H NMR (FIG. 30).

### (e) Synthesis of Polysaccharide-Amino Acid-Lipid (PAL) Polymer

OCMC-S-S-(A/H)- SA Chit-S-S-(A/H)-SA was synthesized by the coupling of the carboxyl group of OCMC-S-S- (A/H) with the amine group of stearyl amine (TCI Chemicals). In brief, the carboxyl group of OCMC-S-S-(A/H) (250 mg) at the C-6 position was activated by the addition of EDC and NHS in 20 mL PBS (pH 6, 10 mM) for two hours. Different amount of stearyl amine (0.25-0.625 mol/mol glucosamine residues) was used to react with —COOH groups of OCMC-S-S-(A/H). The stearyl amine was pre-dissolved in 20 mL ethanol by heating at 60° C. in a separate round bottom flask. After two hours, the stearyl amine solution was added dropwise to the OCMC-S-S-(A/H) polymer solution by maintaining a similar temperature at 60° C. and again heated to 80° C. for the next 6 h. After that, the reaction mixture was allowed to cool to room temperature and again stirred for 18 h. For purification, the reaction mixture was vigorously dialyzed (MWCO 3.5 KDa) against distilled water for 48 h to remove water-soluble by-products and ethanol. The dialyzed suspension was lyophilized and rinsed several times with hot ethanol and diethyl ether and precipitated in ethanol to remove unreacted stearyl amine. Boc deprotection of the amino acids conjugated at the C-2 position of OCMC-S-S-(A/H)-SA (200 mg) was performed using 2 M HCl in dioxane (2 mL) and trifluoroacetic acid (TFA) in ice-cold temperature under an argon atmosphere and stirred for 15 min and further stirred for next three hours at RT. The reaction product was further precipitated in ethanol, washed, and dried. The residue was dialyzed against 0.01 N HCl by redissolving in DI water using dialysis tubing of 3.5 kDa MWCO. The samples were initially dialyzed against 0.01 N HCl for one day and then with DI water for another day with several water changes. I H NMR confirmed the incorporation of stearyl chains in the OCMC-S-S-(A/H) polymer (FIG. 31).

### (11) Characterization Methods

(a) Quantification of free thiol content in polymers:

Elmann's assay To confirm the synthesis of OCMC-S-S-Cys and reduced concentration of free thiols in OCMC-S-S-Cys polymer compared to OCMC-SH, we performed the Elmann's assay of OCMC after and before thiolation as well as after disulfide bond formation. In brief, a reaction buffer was prepared using sodium phosphate 0.1 M and 1 mM ethylenediaminetetraacetic acid (EDTA) in pH 8.0. The stock solution of Elmann's reagent was prepared by dissolving 2 mg of 5,5′-dithiobis-(2-nitrobenzoic acid) in 0.5 mL same reaction buffer. Per the manufacturer's instructions, 500 μL of the sample (mg of polymer in I mL of reaction buffer) was added to a test tube containing 100 pL Elmann's reagent and 5 mL of reaction buffer. The samples were incubated for an optimized time at 37° C. and protected from light. A 100 μl of the sample was transferred to a 96-well plate. Samples were analyzed using a microplate reader (BiOTEK Synergy HT plate reader, Gen5 software) at a wavelength of 485 nm to determine the content of thiol groups. For the estimation of disulfide contents, in OCMC—S—S-Cys polymers were first reduced with NaBH4 and then evaluated by Elmann's reagent. A serial dilution of cysteine hydrochloride monohydrate was used as a standard, and a standard curve is generated using eight serial concentrations of 1.5, 1.25, 1.0, 0.75, 0.5, 0.25, 0.125, 0.0625, and 0 mM. All experiments were performed in triplicate. The free thiol content was quantified according to the following equation:

$$Thiolation \% = \frac{( OCMC … SH - OCMC )}{( OCMC … SH )} \times 100$$

where OCMC and OCMC-SH stands for carboxylated chitosan and thiolated carboxylated chitosan, respectively.

### (b) Nanoparticles Stability

A time-dependent PAL-NPs degradability behavior was evaluated using DTT as a reducing agent. The PAL-NPs (0.5 mg/mL) dispersion with and without disulfide bond was prepared in PBS (10 mM, pH 7.4). The reducing agent dithiothreitol was added to the solution with the final concentration of 10 mM. The samples were incubated at 37° C. and protected from light. At regular time points (0 h, 2 h, 6 h, and 12 h), the particle's average size was measured by DLS (Dynamic Light Scattering). Particle size degradation with respect to the time was plotted. (See FIG. S1c).

### (c) Adjuvant Loading on PAL-NPs

Nanoparticle size and surface zeta potential before the anionic adjuvant loading were measured with a Zetasizer Nano Z. S. (Malvern), as shown in Table 1. The sample preparation details for TEM are provided in section 2.2.5. R848 encapsulation was determined by dissolving PAL-NPs particles in DMSO (Tocris, Cat #3176), followed by absorbance readings against a R848 standard curve at 324 nm. PUUC RNA loading was quantified by Ribogreen assay according to the manufacturer's instructions. CpG DNA loading was quantified by measurement of unbound DNA in the supernatant after centrifugation at 20,000g, using a Nucleic Acid Quantification workflow on a Synergy H.T. plate reader (BioTek) with Gen5 software.

### (d) NMR Spectroscopy

The Iti NMR analysis was performed on a Bruker Avance 111400 at 25° C. OCMC, OCMC-SH, OCMC-S-S-Cys polymers were dissolved in D2O with 1% DCI. OCMC-S-S-(A/H) and OCMC-S-S-(A/H)-SA polymers were dissolved in deuterated dimethyl sulfoxide (DMSO-d6). Chemical shifts were recorded in parts per million (ppm) using the signal of TMS as the internal reference. NMR spectral data were analyzed using MestreNova NMR software. (e) Transmission electronic microscopy (TEM)

TEM was performed on a FEI Tecnai G2 F30 S-TWIN Transmission Electron Microscope at 300 kV. The 10 μL PAL-NPs solution (10 times a diluted sample of0.5 mg/mL) was placed on the copper grids for sample preparation. The excess solution was absorbed by Whatman filter paper at the edges and dried for 10 sec at RT. Samples were further stained by a drop of phosphotungstic acid (stock solution of 2%) onto the surface of the sample-loaded grid. The grid was washed twice with DI water to remove excess staining reagent. Grids were dried in desiccators overnight and analyzed by transmission electron microscopy. (12) immunological studies:

### (a) Euthanasia and Sample Collection (BAL Fluid, Blood, And Lungs)

For IM and IN in vivo studies, mice were euthanized at day 35 (after 2 weeks of booster dose), and blood, BAL fluid, and lungs were harvested. Mice were initially anesthetized using an optimized mixture of ketamine (80 mg/kg) and xylazine (15 mg/kg), injected 25 μl intraperitoneally first and 50 μl intramuscularly later (7-8 minutes later). Blood was first collected from all mice via the jugular veins. All blood samples were allowed to clot for 30-60 min at RT in serum separator tubes (B.D., #365967), and serum was separated by centrifugation at 4000g for 15 min at 4° C. Serum samples were heat inactivated at 56° C. for 30 min in a water bath which inhibits the complement binding. After inactivation, serum samples were aliquoted and stored at −80° C. BAL fluid was collected after two separate injections and withdrawals (total 2 ml in Hanks' Balanced Salt Solution, sigma Aldrich cat #H4641 with 100 μM EDTA Sigma Aldrich cat #03699,) by inserting a 20 gauze one-inch catheter into the trachea. Samples were centrifuged at 300g for 5 minutes to remove cells. BAL Samples were further concentrated 1Ox using 1OOKDa Amicon concentrators and aliquoted and stored at −80° C.

### (b) SARS-CoV-2 RBD B-Cdl Tetramer Production RBD Tetramer was Prepared.

Recombinant Biotinylated SARS-CoV-2 S protein RBD, His, Avitagm (ACRO Biosystems SPD-C82E9) was incubated at a 4:1 molar ratio with either streptavidin-PE (Biolegend, 405204) or streptavidin- APC (Biolegend, 405207) in PEB buffer (1X PBS+0.5% BSA 2 mM EDTA) for one hour at 4° C. The mixture was then purified, concentrated in an Amicon Ultra (50 kDa MWCO) spin column, and washed with sterile, cold PBS. Excess streptavidin was blocked with biotin. Final protein concentration was measured on a nanodrop, using a protein quantification workflow on a Synergy H.T. plate reader (BioTek) with Gen5 software. Tetramers were diluted to 1.0 μM in PBS and stored at 4° C.

### (c) Supernatant Cytokine Profile

Supernatant from the lung T cellrestimulation assay were harvested, and TH I/TH2 cytokine production was measured using LEGENDplex^{l}rm (Mouse TH 1/TH2 Panel, Biolegend, 741054) for IL-5, IL-13, IL-2, IL-6, IL-10, IFN-γ, TNF-α, IL-4, according to manufacturer's instructions. Cytokine beads were analyzed on a cytoflex flow cytometer. Raw data were analyzed using LegendPlex software (Biolegend), and the average cytokine level was determined from two duplicate samples.

### D. References

A. Bemkop-Schniirch, S. Steininger, Synthesis and characterization of mucoadhesive thiolated polymers. International journal of pharmaceutics. 194, 239-247 (2000).
A. R. Bourgonje, A. E. Abdulle, W. Timens, J. Hillebrands, G. J. Navis, S. J. Gordijn, M. C. Boiling, G. Dijkstra, A. A. Voors, A. D. Osterhaus, P. H. Voort, D. J. Mulder, H. Goor, Angiotensin-converting enzyme 2 (ACE2), SARS-CoV-2 and the pathophysiology of coronavirus disease 2019 (COVID-19). J Pathol. 251, 22&..-248 (2020).
A. S. Agrawal, X. Tao, A. Algaissi, T. Garron, K. Narayanan, B.-H. Peng, R. B. Couch, C.- T. K. Tseng, Immunization with inactivated middle east respiratory syndrome coronavirus vaccine leads to lung immunopathology on challenge with live virus. Hum Vaccin Inununother. 12, 2351—2356 (2016).
A. Alu, L. Chen, H. Lei, Y. Wei, X. Tian, X. Wei, Intranasal COVID-19 vaccines: from bench to bed. EBioMedicine. 76, 103841 (2022).
A. Atalis, M.C. Keenum, B. Pandey, A. Beach. P. Pradhan, C. Vantucci, L. O'Farrell, R. Noel, R. Jain, J. Hosten. C. Smith, L. Kramer, A. Jimenez, M. A. Ochoa, D. Frey, K. Roy, Nanopanicle-delivered TLR4 and RIG-I agonists enhance immune response to SARS-CoV- 2 subunit vaccine. Journal of Controlled Release. 347, 476-488 (2022).
A. Grifoni, D. Weiskopf, S. I. Ramirez, J. Mateus, J. M. Dan, C. R. Moderbacher, S. A. Rawlings, A. Sutherland, L. Premkumar, R. S. Jadi, D. Marrama, A. M. de Silva, A. Frazier, A. F. Carlin, J. A. Greenbaum, B. Peters, F. Krammer, D. M. Smith, S. Crotty, A. Sette, Targets of T cell responses to SARS-CoV-2 coronavirus in humans with COVID-19 disease and unexposed individuals. Cell. 181, 1489-1501,e15 (2020).
A. Iwasaki, Exploiting mucosal immunity for antiviral vaccines. Annu Rev Immunol. 34, 575-608 (2016).
A. Purwada, K, Roy, A. Singh. Engineering vaccines and niches for immune modulation. Acta Biomater. 10, 1728-1740 (2014).
B. lsraelow, T. Mao, J. Klein, E. Song, B. Menasche, S. B. Omer, A. Iwasaki, Adaptive immune determinants of viral clearance and protection in mouse models of SARS-CoV-2. Sci Immunol. 6, eab14509 (2021).
B. Pulendran, P. S. Arunachalam, D. T. O'Hagan, Emerging concepts in the science of vaccine adjuvants. Nat Rev Drug Discov. 20, 454-475 (2021).
B. Zhou, V. A. Meliopoulos, W. Wang, X. Lin, K. M. Stucker, R. A. Halpin, T. B. Stockwell. S. Schultz-Cherry, D. E. Wentworth, reversion of cold-adapted live attenuated influenza vaccine into a pathogenic virus. J Virol. 90, 8454-8463 (2016).
D. S. Khoury, D. Cromer, A. Reynaldi, T. E. Schlub, A. K. Wheatley, J. A. Juno, K. Subbarao, S. J. Kent, J. A. Triccas, M. P. Davenport, Neutralizing antibody levels are highly predictive of immune protection from symptomatic SARS-CoV-2 infection. Nat Med. 27, 1205-1211 (2021).
D. Christensen, C. Polacek, D. J. Sheward, L. Hanke, A. Moliner-Morro, G. McInerney, B. Murrell, K. T. Hartmann, H. E. Jensen, G. Jungersen, K. Ililigen, L. K. Isling, R. F. Jensen, J. S. Hansen, I. Rosenkrands, C. Fernandez-Antunez, S. Ramirez, F. Follmann, J. Bukh, G. K. Pedersen, Protection against SARS-CoV-2 transmission by a parenteral prime- Intranasal boost vaccine strategy. EBioMedicine. 84, 104248 (2022).
D. Cibrian, F. Sanchez-Madrid, CD69: from activation marker to metabolic gatekeeper. Eur J Immunol. 47, 946-953 (2017).
D. Cromer, M. Steain, A. Reynaldi, T. E. Schlub, A. K. Wheatley, J. A. Juno. S. J. Kent, J. A. Triccas, D. S. Khoury, M. P. Davenport, Neutralising antibody titers as predictors of protection against SARS-CoV-2 variants and the impact of boosting: a meta-analysis. Lancet Microbe. 3, e52-e61 (2022).
D. Focosi, F. Maggi, A. Casadevall, Mucosal vaccines, sterilizing immunity, and the future of SARS-CoV-2 virulence. Viruses. 14, 187 (2022).
D. Wibowo, S. H. T. Jorritsma, Z. J. Gonzaga, B. Evert, S. Chen, B. 1. A. Rehm, Polymeric nanoparticle vaccines to combat emerging and pandemic threats. Biomaterials. 268, 120597 (2021).
E. Waltz, How nasal-spray vaccines could change the pandemic. Nature. 609, 240-242 (2022).
E.G. Levin, Y. Lustig, C. Cohen, R. Fluss, V. Indenbaum, S. Amit, R. Doolman, K. Asraf, E. M endelson, A. Ziv, C. Rubin. L. Freedman. Y. Kreiss, G. Regev-Yochay, Waning Immune Humoral Response to BNT162b2 Covid-19 Vaccine over 6 Months. New England Journal of Medicine. 385, e84 (2021).
H. W. Kim, J. G. Canchola, C. D. Bradnt, G. Pyles, R.. M. Chanock, K. Jensen, R. H. Parrot, Respiratory Syncytial Virus disease in infants despite prior administration of antigenic inactivated vaccine. Am J Epidemiol. 89, 422-434 (1969).
H. Y. Yong, D. Luo, RIG-I-like receptors as novel targets for pan-antivirals and vaccine adjuvants against emerging and re-emerging viral infections. Front Immunol. 9, (2018).
H. Lei, A. Alu, J. Yang, C. He, W. Hong, Z. Cheng, L. Yang, J. Li. Z Wang, W. Wang, G. Lu, X. Wei, Cationic nanocarriers as potent adjuvants for recombinant S-RBD vaccine of SARS-CoV-2. Signal Transduct Target Ther. 5, 291(2020).
H. Lei, A. Alu, J. Yang, W. Ren, C. He, T. Lan, X. He, L. Yang, J. Li, Z. Wang, X. Song, W. W ang, G. Lu, X. Wei, Intranasal administration of a recombinant RBD vaccine induces long-term immunity against Omicron-included SARS-CoV-2 variants. Signal Transduct Target Ther. 7, 159 (2022).
J. H. Park, H. K. Lee, Delivery routes for COVID-19 vaccines. Vaccines (Basel). 9, 524 (2021).
J. M. E. Lim, A. T. Tan, N. Le Bert, S. K. Hang, J. G. H. Low, A. Bertoletti, SARS-CoV-2 breakthrough infection in vaccinees induces virus-specific nasal-resident CD8⁺ and CD4⁺ T cells of broad specificity. Journal of Experimental Medicine. 219, e20220780 (2022).
J. M. Brewer, M. Conacher, C. A. Hunter, M. Mohrs, F. Brombacher, J. Alexander, Aluminium hydroxide adjuvant initiates strong antigen-specific TH2 responses in the absence of IL-4- or IL-13-mediated signaling. J Immunol. 163, 6448-54 (1999).
J. M. Knisely, L. E. Buyon, R. Mandt, R. Farkas, S. Balasingam, K. Bok, U. J. Buchholz, M. P. D'Souza, J. L. Gordon, D. F. L King, T. T. Le, W. W. Leitner, R. A. Seder, A. Togias, S. Tollefsen, D. W. Vaughn, D. N. Wolfe, K. L. Taylor, A. S. Fauci, Mucosal vaccines for SARS-CoV-2: scientific gaps and opportunities-workshop report. NPJ Vaccines. 8, 53 (2023).
J. S. Errett, M. S. Suthar, A. McMillan, M. S. Diamond, M. Gale, The essential, nonredundant roles of RIG-1 and MDAS in detecting and controlling west nile virus infection. J Virol. 87, 11416-11425 (2013).
J. Helft, J. Bottcher, P. Chakravarty, S. Zelenay, J. Huotari, B.U. Schraml, D. Goubau, C. Reis e Sousa, GM-CSF mouse bone marrow cultures comprise a heterogeneous population of CDI IcMHC1 macrophages and dendritic cells. Immunity. 42, 1197-1211 (2015).
J. Jameson, J. Cruz, F. A. Ennis, Human Cytotoxic T-lymphocyte repertoire to influenza A viruses. J Virol. 72, 8682—-8689 (1998).
J. Tang, C. Zeng, T. M. Cox, C. Li, Y. M. Son, I. S. Cheon, Y. Wu, S. Behl, J. J. Taylor, R. Chakaraborty, A. J. Johnson, D. N. Shiavo, J. P. Utz, J. S. Reisenauer, D. E. Midthun, J. J. Mullon, E. S. Edell, M. G. Alameh, L Borish, W. G. Teague, M. H. Kaplan, D. Weissman, R. Kern, H. Hu, R. Vassallo, S.-L. Liu, J. Sun, Respiratory mucosal immunity against SARS-CoV-2 after mRNA vaccination. Sci Immunol. 7, eadd4853 (2022).
J. Volckmar, L. Melcher, D. Bruder, Route, origin & valence matter: towards sophisticated next-generation vaccines to cope with the COVID-19 pandemic. Signal Transduct Target Ther. 7, 188 (2022).
J. Zhu, S. Jain, J. Sha, H. Batra, N. Ananthaswamy, P. B. Kilgore, E. K. Hendrix, Y. M. Hosakote, X. Wu, J. P. Olano, A. Kayode, C. L. Galindo, S. Banga, A. Drelich, V. Tat, C.- T. K. Tseng, A. K. Chopra, V. B. Rao, A bacteriophage-based, highly efficacious, needle- and adjuvant-free, mucosal COVID-19 vaccine. mBio. 13 (2022).
K. G. Nguyen, S. M. Mantooth, M. R. Vrabel, D. A. Zaharoff, Intranasal delivery of thermostable subunit vaccine for cross-reactive mucosal and systemic antibody responses against SARS-CoV-2. Front immunol. 13 (2022).
K. J. Siddle, L. A. Krasilnikova, G. K. Moreno, S. F. Schaffner, J. Vostok, N. A. Fitzgerald, J. E. Lemieux, N. Barkas, C. Loreth, I. Specht, C. H. Tomkins-Tinch, J. S. Paull, B. Schaeffer, B. P. Taylor, B. Loftness, H. Johnson, P. L. Schubert, H. M. Shephard, M. Doucette, T. Fink, A. S. Lang, S. Baez, J. Beauchamp, S. Hennigan, E. Buzby, S. Ash, J. Brown, S. Clancy, S. Cofsky, L. Gagne, J. Hall, R. Harrington, G. L. Gionet, K. C. DeRuff, M. E. Vodzak, G. C. Adams, S. T. Dobbins, S. D. Slack, S. K. Reilly, L. M. Anderson, M. C. Cipicchio, M. T. DeFelice, J. L. Grimsby, S. E. Anderson, B. S. Blumenstiel, J. C. Meldrim, H. M. Rooke, G. Vicente, N. L. Smith, K. S. Messer, F. L. Reagan, Z. M. Mandese, M. D. Lee, M. C. Ray, M. E. Fisher, M. A. Ulcena, C. M. Nolet, S. E. English, K. L. Larkin, K. Vernest, S. Chaluvadi, D. Arvidson, M. Melchiono, T. Covell, V. Harik, T. Brock-Fisher, M. Dunn, A. Kearns, W. P. Hanage, C. Bernard, A. Philippakis, N. J. Lennon, S. B. Gabriel, G. R. Gallagher, S. Smole, L. C. Madoff, C. M. Brown, D. J. Park, B. L. MacInnis, P. C. Sabeti, Transmission from vaccinated individuals in a large SARS- CoV-2 Delta variant outbreak. Cell. 185, 485—492.e10 (2022).
K. Zhao, Y. Xie, X. Lin, W. Xu, The mucoadhesive nanoparticle-based delivery system in the development of mucosal vaccines. Int J Nanomedicine. 17, 4579-4598 (2022).
L. R. Baden, H. M. El Sahly, B. Essink, K. Kotloff, S. Frey, R. Novak, D. Diemert, S. A. Spector, N. Rouphael, C. B. Creech, Efficacy and safety of the mRNA-1273 SARS-CoV-2 vaccine. New England Journal of Medicine. 384, 403-416 (2021).
L. V. Hoecke, E. R. Job, X. Saelens, K. Roose, Bronchoalveolar lavage of murine
L. Du, Y. He, S. Jiang, B.-J. Zheng, Development of subunit vaccines against severe acute respiratory syndrome. Drugs Today (Barci. 44, 63-73 (2008).
M. M. L. Poon, K. Rybkina, Y. Kato, M. Kubota, R. Matsumoto, N. 1. Bloom, Z. Zhang, K. M. Hastie, A. Grifoni, D. Weiskopf, SARS-CoV-2 infection generates tissue-localized immunological memory in humans. Sci Immunol. 6, eabl9105 (2021).
M. N. Vu, 1-1. G. Kelly, S. J. Kent, A. K. Wheatley, Current and future nanoparticle vaccines for COVID-19. EBioMedicine. 74, 103699 (2021).
M. Deng, Z. Hu, H. Wang, F. Deng, Developments of subunit and VLP vaccines against influenza a virus. Virol Sin. 27, 145-153 (2012).
M. Sadarangani, A. Marchant, T. R. Kollmann, Immunological mechanisms of vaccine- induced protection against COVID-19 in humans. Nat Rev Immunol. 21, 475-484 (2021).
M. Weber-Gerlach, F. Weber. Standing on three legs: antiviral activities of RIG-4 against influenza viruses. Curr Opin Immunol. 42, 71-75 (2016).
N. C. Kyriakidis, A. L6pez-Cortes, E. V. Gonzalez, A. B. Grimaldos, E. O. Prado, SARS- CoV-2 vaccines strategies: a comprehensive review of phase 3 candidates. NPJ Vaccines. 6, 28 (2021).
N. K. Routhu, N. Cheedarla, V. S. Bollimpelli, S. Gangadhara, V. V. Edara, L. Lai, A. Sahoo, A. Shiferaw, T. M. Styles, K. Floyd, S. Fischinger, C. Atyeo, S. A. Shin, S. Gumber, S. Kirejczyk, K. H. Dinnon, P.-Y. Shi, V. D. Menachery, M. Tomai, C. B. Fox, G. Alter, T. H. Vanderford, L. Gralinski, M. S. Suthar, R. R. Amara, SARS-CoV-2 RBD trimer protein adjuvanted with Alum-3M-052 protects from SARS-CoV-2 infection and immune pathology in the lung. Nat Commun. 12, 3587 (2021).
N. Dagan, N. Barda, E. Kepten, 0. Miron, S. Perchik, M. A. Katz, M. A. Hernin, M. Lipsitch, B. Reis, R. D. Balicer, BNT162b2 mRNA Covid-19 vaccine in a nationwide mass vaccination setting. New England Journal of Medicine. 384, 1412-1423 (2021).
N. Lycke, Recent progress in mucosal vaccine development: potential and limitations. Nat Rev Immunol. 12, 592-605 (2012).
N. van Doremalen, J. N. Purushotham, J. E. Schulz, M. G. Holbrook, T. Bushmaker, A. Carmody, J. R. Port, C. K. Yinda, A. Okumura, G. Saturday, F. Amanat, F. Krammer, P. W. Hanley, B. J. Smith, J. Lovaglio, S. L. Anzick, K. Barbian, C. Martens, S. C. Gilbert, T. Lambe, V. J. Munster, Intranasal ChAdOxI nCoV-19/AZD1222 vaccination reduces viral shedding after SARS-CoV-2 D614G challenge in preclinical models. Sci Transl Med. 13, eabh0755 (2021).
N. Zhang, K. Li, Z. Liu, K. S. Nandakumar, S. Jiang, A perspective on the roles of adjuvants in developing highly potent COVID-19 vaccines. Viruses. 14, 387 (2022).
P. C. Rosato, L. K. Beura, D. Masopust, Tissue resident memory T cells and viral immunity. Curr Opin Virol. 22, 44-50 (2017).
P. Pradhan, R. Toy, N. Jhita, A. Atalis, B. Pandey, A. Beach, E. L. Blanchard, S. G. Moore, D. A. Gaul, P. J. Santangelo, D. M. Shayakhmetov, K. Roy, TRAF6-IRFS kinetics, TRIF, and biophysical factors drive synergistic innate responses to particle-mediated MPLA-CpG co-presentation. Sci Adv. 7, eabd4235 (2021).
R. B. Belshe, K. M. Edwards, T. Vesikari, S. V. Black, R. E. Walker, M. Hultquist, G. Kemble, E. M. Connor. Live Attenuated versus Inactivated Influenza Vaccine in Infants and Young Children. New England Journal of Medicine. 356, 685-696 (2007).
R. J. Fischer, N. van Doremalen, D. R. Adney, C. K. Yinda, J. R. Port, M. G. Holbrook, J. E. Schulz, B. N. Williamson, T. Thomas, K. Barbian, ChAdOxI nCoV-19 (AZD1222) protects Syrian hamsters against SARS-CoV-2 B. 1.351 and B. 1.1. 7. Nat Commun. 12, 1-11 (2021).
R. K. Gupta, E. J. Topol, COVID-19 vaccine breakthrough infections. Science. 374, 1561——1562 (2021).
R. L. Coffman, A. Sher, R. A. Seder, Vaccine adjuvants: putting innate immunity to work. Immunity. 33, 492-503 (2010).
R. Toy, M. C. Keenum, P. Pradhan, K. Phang, P. Chen, C. Chukwu, L. A. H. Nguyen, J. Liu, S. Jain, G. Kozlowski, J. Hosten, M. S. Suthar, K. Roy, TLR7 and RIG-I dual-adjuvant loaded nanoparticles drive broadened and synergistic responses in dendritic cells in vitro and generate unique cellular immune responses in influenza vaccination. Journal of Controlled Release. 330, 866-877 (2021).
R. Toy, P. Pradhan, V. Ramesh, N. C. Di Paolo, B. Lash, J. Liu, E. L. Blanchard, C. J. Pinelli, P. J. Santangelo, D. M. Shayakhmetov, K. Roy, Modification of primary amines to higher order amines reduces in vivo hematological and immunotoxicity of cationic nanocarriers through TLR4 and complement pathways. Biomaterials. 225, 119512 (2019).
S. G. Reed, M. T. Orr, C. B. Fox, Key roles of adjuvants in modem vaccines. Nat Med. 19, 1597-1608 (2013).
S. R. Allie, J. E. Bradley, U. Mudunuru, M. D. Schultz, B. A. Graf, F. E. Lund, T. D. Randall, The establishment of resident memory B cells in the lung requires local antigen encounter. Nat Immunol. 20, 97-108 (2019).
S. R. Holdsworth, A. R. Kitching, P. G. Tipping, TH1 and TH2 T helper cell subsets affect patterns of injury and outcomes in glomerulonephritis. Kidney Int. 55, 1198-1216 (1999).
S. Cele, L. Jackson, D. S. Khoury, K. Khan, T. Moyo-Gwete, H. Tegally, J. E. San, D. Cromer, C. Scheepers, D. Amoako, SARS-CoV-2 Omicron has extensive but incomplete escape of Pfizer BNTI62b2 elicited neutralization and requires ACE2 for infection. Nature. 602, 654-656 (2021).
S. Havervall. U. Marking, J. Svensson, N. Greilert-Norin, P. Bacchus, P. Nilsson, S. Hober, M. Gordon, K. Blom, J. Klingstrom, M. Aberg, A. Sme d-S6rensen, C. Thflin, Anti-Spike Mucosal IgA protection against SARS-CoV-2 omicron infection. New England Journal of Medicine. 387, 1333-1336 (2022).
S. Jangra, J. J. Landers, R. Rathnasinghe, J. J. O'Konek, K. W. Janczak. M. Cascalho, A. A. Kennedy, A. W. Tai, J. R. Baker, M. Schotsaert, P. T. Wong, A combination adjuvant for the induction of potent antiviral immune responses for a recombinant SARS-CoV-2 protein vaccine. Front Immunol. 12, (20211.
S. Wu, G. Zhong, J. Zhang, L. Shuai, Z. Zhang, Z. Wen, B. Wang, Z. Zhao, X. Song, Y. Chen, R Liu, L, Fu, J. Zhang, Q. Guo, C. Wang, Y. Yang, T. Fang, P. Lv, J. Wang, J. Xu, J. Li, C. Yu, L Hou, Z. Bu. W. Chen, A single dose of an adenovirus-vectored vaccine provides protection against SARS-CoV-2 challenge. Nat Commun. 11, 4081 (2020).
T. L. Bricker, T. L. Darling, A. O. Hassan, H. H. Harastani, A. Soung, X. Jiang, Y.-N. Dai, H. Zhao, L. J. Adams, M. J. Holtzman, A single intranasal or intramuscular immunization with chimpanzee adenovirus-vectored SARS-CoV-2 vaccine protects against pneumonia in hamsters. Cell Rep. 36, 109400 (2021).
T. Y. Wong, K. S. Lee, B. P. Russ, A. M. Horspool, J. Kang, M. T. Winters, M. Allison Wolf N. A. Rader, O. A. Miller, M. Shiflett, J. Izac, D. Varisco, E. Sen-Kilic, C. Cunningham, M. Cooper, H. A. Cyphert, M. Barbier, I. Martinez, J. R. Bevere, R. K. Ernst, F. H. Damron, intranasal administration of BReC-CoV-2 COVID-19 vaccine protects K18- hACE2 mice against lethal SARS-CoV-2 challenge. NPJ Vaccines. 7, 36 (2022).
T. Y. Wong, K. S. Lee, B. P. Russ, A. M. Horspool, J. Kang, M. T. Winters, M. Allison Wolf, N. A. Rader, O. A. Miller, M, Shiflett, J. Izac, D. Varisco, E. Sen-Kilic, C. Cunningham, M. Cooper, H. A. Cyphert, M. Barbier, I. Martinez, J. R. Bevere, R.. K. Ernst, F. H. Damron. Intranasal administration of BReC-CoV-2 COVID-19 vaccine protects K18- hACE2 mice against lethal SARS-CoV-2 challenge. NPJ Vaccines. 7, 36 (2022).
T. Mao, B. lsraelow, M. A. Pedla-Hernandez, A. Suberi, L. Zhou, S. Luyten, M. Reschke, H. Doug, R. J. Homer, W. M. Saltzman, A. Iwasaki, Unadjuvanted intranasal spike vaccine elicits protective mucosal immunity against sarbecoviruses. Science. 378 (2022).
U. Sahin, A. Muik, E. Derhovanessian, 1. Vogler, L. M. Kranz, M. Vormehr, A. Baum, K. Pascal, J. Quandt, D. Maurus, COVID-19 vaccine BNT162bl elicits human antibody and TH1 T cell responses. Nature. 586, 594——599 (20201.
V. Appay, The physiological role of cytotoxic CD4⁺ T-cells: the holy grail?Clin Exp Immunol. 138, 10-13 (2004).
V. Beljanski, C. Chiang, G. A. Kirchenbaum, D. Olagnier, C. E. Bloom, T. Wong, E. K. Haddad, L. Trautmann, T. M. Ross, J. Hiscott, Enhanced influenza virus-like particle vaccination with a structurally optimized RIG-I agonist as adjuvant. J Virol. 89, 10612-10624 (2015).
W. C. Koff. T. Schenkelberg, T. Williams, R. S. Baric, A. McDermott, C. M. Cameron, M. J. Cameron, M. B. Friemann, G. Neumann, Y. Kawaoka, A. A. Kelvin. T. M. Ross, S. Schultz-Cherry, T. D. Mastro, F. H. Priddy, K. A. Moore, J. T. Ostrowsky, M. T. Osterholm, J. Goudsmit, Development and deployment of COVID-19 vaccines for those most vulnerable. Sci Transl Med. 13, 579 (2021).
X. G. Chen, H. J. Park, Chemical characteristics of O-carboxymethyl chitosans related to the preparation conditions. Carbohydrate Polymers, 53, 355-359 (2003).
X. Huang, E. Kon, X. Han, X. Zhang, N. Kong, M. J. Mitchell, D. Peer, W. Tao, Nanotechnology-based strategies against SARS-CoV-2 variants. Nat Nanotechnol. 17, 1027-1037 (2022).
Y. M. Son, 1. S. Cheon, Y. Wu, C. Li, Z. Wang, X. Gao, Y. Chen, Y. Takahashi, Y.-X. Fu, A. L. Dent, M. H. Kaplan, J. J. Taylor, W. Cui, J. Sun, Tissue-resident CD4⁺ T helper cells assist the development of protective respiratory B and CD8°⁺ T cell memory responses. Sci Immunol. 6, eabb6852 (2021).
Y. M. Son, J. Sun, Co-ordination of mucosal B Cell and CD8 T cell memory by tissue- resident CD4 helper T cells. Cells. 10, 2355 (2021).
Y. Wang, L. Wang, H. Cao, C. Liu, SARS-CoV-2 S1 is superior to the RBD as a COVID- 19 subunit vaccine antigen. J Med Virol. 93, 892-898 (2021).
Z. Wang, F. Schmidt, Y. Weisblum, F. Muecksch, C. O. Barnes, S. Finkin, D. Schaefer-Babajew, M. Cipolla, C. Gaebler, J. A. Lieberman, mRNA vaccine-elicited antibodies to SARS-CoV-2 and circulating variants. Nature. 592, 616-622 (2021).

What is claimed is:

1. A nanoparticle comprising a polymer having an outer surface and an inner core, wherein the polymer comprises a polysaccharide, a lipid, and an amino acid, wherein the lipid and amino acid are conjugated to the polysaccharide.

2. The nanoparticle of claim 1, wherein the polysaccharide is chitosan.

3. The nanoparticle of claim 2, wherein the amino acid is conjugated to a C-2 carbon in chitosan.

4. The nanoparticle of any one of claims 1-3, wherein the amino acid conjugation occurs via disulfide bond.

5. The nanoparticle of any one of claims 2-4, wherein the lipid is conjugated to a C-6 carbon in chitosan.

6. The nanoparticle of any one of claims 1-5, wherein the outer surface of the nanoparticle is hydrophilic.

7. The nanoparticle of any one of claims 1-6, wherein the amino acid comprises arginine and/or histidine.

8. The nanoparticle of any one of claim 7, wherein histidine comprises from 0% to 100% by weight of the total amino acids.

9. The nanoparticle of any one of claim 7, wherein arginine comprises from 0% to 100% by weight of the total amino acids.

10. The nanoparticle of any one of claims 1-9, wherein the outer surface of the nanoparticle is loaded with a first agent.

11. The nanoparticle of claim 10, wherein the first agent comprises a nucleic acid, a polynucleotide, peptide, protein, a siRNA molecule, a miRNA molecule, a shRNA molecule, a pDNA molecule, or any combination thereof.

12. The nanoparticle of any one of claims 10-11, wherein the first agent comprises RIG I, CpG, PUUC, and/or Poly I:C.

13. The nanoparticle of any one of claims 1-12, wherein the inner core of the nanoparticle is hydrophobic.

14. The nanoparticle of any one of claims 1-13, wherein the inner core of the nanoparticle is loaded with a second agent.

15. The nanoparticle of claim 14, wherein the second agent comprises a small molecule, immune adjuvants, fluorochrome, contrast agents, a nucleic acid, a polynucleotide, peptide, protein, a siRNA molecule, a miRNA molecule, a shRNA molecule, a pDNA molecule, or any combination thereof.

16. The nanoparticle of claim 14-15, wherein the second agent is hydrophobic.

17. The nanoparticle of any one of claims 14-16, wherein the second agent comprises R848 and/or MPLA.

18. The nanoparticle of any one of claims 1-17, wherein the nanoparticle is from 10 nm to 1000 nm.

19. The nanoparticle of any one of claims 1-18, wherein the nanoparticle is from 100-300 nm.

20. The nanoparticle of any one of claims 1-19, wherein the nanoparticle has a zeta potential of from +10 mV to +90 mV.

21. The nanoparticle of any one of claims 1-20, wherein the nanoparticle has a zeta potential of from +30 mV to +37 mV.

22. A vaccine comprising the nanoparticle of any one of claims 1-21 and one or more immunogenic nucleic acids, polynucleotide, peptides, antibody, protein, inactivated virus, killed virus, viral particle, or any combination thereof.

23. The vaccine of claim 22, wherein the vaccine comprises a single immunogenic nucleic acid, polynucleotide, peptide, protein, antibody, viral particle, inactivated virus, or killed virus.

24. The vaccine of claim 22, wherein the vaccine comprises more than one immunogenic nucleic acids, polynucleotides, peptides, proteins, antibodies, viral particles, inactivated viruses, and/or killed viruses, or any combination thereof.

25. The vaccine of claim 24, wherein the more than one immunogenic nucleic acids, polynucleotides, peptides, proteins, antibodies, viral particles, inactivated viruses, and/or killed viruses are immunogenic against a first epitope.

26. The vaccine of claim 24, wherein the more than one immunogenic nucleic acids, polynucleotides, peptides, proteins, antibodies, viral particles, inactivated viruses, and/or killed viruses are immunogenic against a first epitope and at least one second epitope.

27. The vaccine of claim 26, wherein the first epitope and the second epitope are the same.

28. The vaccine of claim 26, wherein the first epitope and the second epitope are different.

29. An antimicrobial treatment regimen comprising administering one or more vaccines of any of claims 22-28 and/or one or more of the nanoparticle of any one of claims 1-21 and a vaccine.

30. The antimicrobial treatment regimen of claim 29, wherein the vaccine comprises one or more immunogenic nucleic acids, polynucleotides, peptides, proteins, antibodies, inactivated viruses, killed viruses, viral particles, or any combination thereof

31. The antimicrobial treatment regimen of claim 29 or 30, wherein treatment regimen comprises the administration at least two vaccines, a first vaccine and a second vaccine.

32. The antimicrobial treatment regiment of claim 25, wherein the vaccine comprises a single immunogenic nucleic acid, polynucleotide, peptide, protein, antibody, viral particle, inactivated virus, or killed virus.

33. A method of treating a pulmonary infection, comprising administering a therapeutically effective amount of the nanoparticle of any one of claims 1-21, vaccine of any of claims 22-28, or treatment regimen of any of claims 29-32 to a patient in need thereof.

34. The method of claim 33 or the treatment regimen of any of claims 29-32, wherein the nanoparticle is administered via an intramuscular route, an oral route, an intranasal route, or any combination thereof.

35. The method of any one of claims 33 or 34, wherein the pulmonary infection is SARS-CoV-2.

36. A method of making the nanoparticle in any one of claims 1-21, comprising:
a. carboxylating the polysaccharide;
b. thiolating the polysaccharide;
c. forming disulfide with a cysteamine;
d. conjugating the amino acid using carbodiimide chemistry;
e. conjugating stearyl amine using carbodiimide chemistry;
f. deprotecting a tert-Butyloxycarbonyl group with trifluoroacetic acid;
g. sonicating the nanoparticle; and
h. purifying the nanoparticle with dialysis.

37. The method of claim 36, further comprising loading the nanoparticle with the second therapeutic agent.
